(19) United States
(12) Patent Application Publication
(10) Pub. No.: US 2023/0263872 A1
(43) Pub. Date: Aug. 24, 2023

(54) NEOANTIGENS, METHODS AND DETECTION OF USE THEREOF

(71) Applicant: Envisagenics, Inc., New York, NY (US)

(72) Inventors: Maria Luisa Pineda, New York, NY (US); Martin Akerman, Huntington, NY (US); Gayatri Arun, Huntington Station, NY (US); Naomi Yudanin, New York, NY (US); Priyanka Dhingra, Jersey City, NJ (US)

(73) Assignees: Envisagenics, Inc., New York, NY (US); Envisagenics, Inc., New York, NY (US)

(21) Appl. No.: 18/023,674

(22) PCT Filed: Aug. 27, 2021
(86) PCT No.: PCT/US2021/048073

§ 371 Date: Feb. 27, 2023

Related U.S. Application Data
(60) Provisional application No. 63/071,516, filed on Aug. 28, 2020.

(51) Int. Cl. A61K 39/00; C07K 14/47; A61P 35/00; G16B 15/00; G16B 40/20; G01N 33/50; G16H 50/50

(52) U.S. Cl. CPC A61K 39/0011; A61K 39/4611; A61K 39/4632; A61K 39/4631; C07K 14/4748; A61P 35/00; G16B 15/00; G16B 40/20; G01N 33/5011; G16H 50/50

(57) ABSTRACT

Provided herein are systems and methods for identifying alternative splicing derived cell surface antigens. Also provided are methods and compositions for using the identified cell surface antigens. Further provided are methods, compositions, and systems for diagnosing diseases in a subject using the identified cell surface antigens or treating diseases using the same.

### CROSS-REFERENCE TO RELATED APPLICATIONS

This application claims the benefit of and priority to U.S. Provisional Patent Application No. 63/071,516, filed on Aug. 28, 2020, the disclosure of which is hereby incorporated by reference in its entirety for all purposes.

### STATEMENT AS TO FEDERALLY SPONSORED RESEARCH

This invention was made with U.S. government support, Grant No. 1R43CA246950-01, awarded by National Institute of Health under the Department of Health and Human Services. The U.S. government has certain rights to the invention.

### FIELD OF THE INVENTION

The invention relates generally to methods and compositions of alternative splicing derived cell surface antigens and their use, e.g., for treating disease.

### BACKGROUND

Immunotherapeutics are driving cancer treatment innovation with a number of immune check point inhibitors and adoptive cell transfer technologies currently in clinical trials, a subset of which has now obtained FDA approval (e.g., Pembrolizumab, Nivolumab, Ipilimumab). However, immunotherapies are currently limited in two ways: first, in their selective response and consequent success in only 30-40% of recipients. Second, their use is limited to cancers with high tumor mutational burdens (TMB) (e.g., melanoma and lung cancer), microsatellite instability (MSI) (e.g., colon cancer), neoantigen expression, and limited immune suppression. As a result, immunotherapies are ineffective in a significant proportion of tumor types (e.g. breast, pancreatic, hepatic, gastric cancer etc.). Neoantigens, novel proteins and peptides derived from mutations and alternative splicing events in cancer cells can be targeted with immunotherapeutic agents. However, difficulties in targeting these cancers stem from limited utility of neoantigen detection through the Whole Exome Sequencing (WES) based approach. RNA-seq data can be used to characterize such alterative splicing events. Accordingly, new methods for data analysis of RNA-seq data to characterize alternative splicing events and discover neoantigens are needed.

### SUMMARY OF THE INVENTION

Alternative splicing of mRNA and its resulting mRNA transcripts and protein isoforms are associated with many diseases such as cancer. In one aspect, the disclosure provides systems and methods for identifying cell surface antigen sequences resulting from alternative splicing in a cell that are likely to be presented on the surface of the cell. In another aspect, the disclosure provides for cell surface antigen sequences derived from alternative splicing events, therapeutical compositions and methods of treatment for subjects with alternative splicing associated disease.

In one aspect, the disclosure provides computer-implemented systems and methods for identifying one or more cell surface antigen sequences resulting from alternative splicing in a cell, comprising the steps of: obtaining a first RNA-seq data set from a first sample cell and a second RNA-seq data set from a second sample cell; assembling full length mRNA transcript sequences and extracting genomic loci coordinates of the mRNA transcript sequences; clustering of full length mRNA transcript sequences encoded at the same genomic loci and extraction of exon duo or exon trio mRNA sequences; selecting the most representative full length mRNA transcript sequences; identifying stable full length mRNAs transcripts; translating, in silico the stable full length mRNA transcripts into protein isoform sequences; identifying protein isoform sequences that are predicted to be stable; determining B cell antibody accessibility of the protein isoform sequences by using an algorithm to classify the polarity, hydrophobicity, and surface accessibility of peptides derived from the protein isoform sequences; determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics 0) responsive or non-responsive, and/or (ii) antigenic or non-antigenic; generating a first set of antigenic cell surface antigen sequences based on the first RNA-seq data set and a second set of antigenic cell surface antigen sequences based on the second RNA-seq data set ranked by B cell antibody accessibility and T cell antigenicity and determining unique antigenic cell surface antigen sequences by comparing the first set of antigenic cell surface antigen sequences and the second set of antigenic cell surface antigen sequences and selecting cell surface antigen sequences present in one set and not the other set; thereby selecting one or more unique cell surface antigen sequences. In some embodiments, the method further comprises determining membrane topologies for each protein isoform sequence and filtering for membrane bound protein isoform sequences.

In some embodiments, the machine learning algorithm is semi-supervised or supervised machine learning algorithm and comprises: a random forest, Bayesian model, a regression model, a neural network, a classification tree, a regression tree, discriminant analysis, a k-nearest neighbors method, a naive Bayes classifier, support vector machines (SVM), a generative model, a low-density separation method, a graph-based method, a heuristic approach, or a combination thereof. In some embodiments the machine learning algorithm comprises a random forest algorithm. In some embodiments, semi-supervised or supervised machine learning algorithm used to classify the membrane topology of the protein isoform is trained using a training data set comprising training protein sequences encoded with two characteristics i) transmembrane brane or globular or ii) with signal peptide or without signal peptide. In some embodiments, the training peptide sequences comprise peptide sequences having lengths from 5 to 25 amino acids or 8 to 15 amino acids. In some embodiments, the training peptide sequences are of viral and bacterial origin.

In some embodiments, the cell surface antigen is derived from alternative splicing events for example intron retention, frameshift, translated lncRNA, novel splicing junction, novel exon, and chimeric.

In some embodiments, cell surface antigen sequences that have an increased likelihood of being presented on the tumor cell surface relative to unselected cell surface antigen sequences can be selected.

In some embodiments, the method further comprises determining if the cell surface antigen cell surface presentation is MHC-dependent or MHC-independent. In some embodiments, the cell surface presentation of the cell surface antigen derived peptide is MHC-independent.

In some embodiments, the first or second cell is a cancer cell. The cancer cell can be for example a bone cancer, a breast cancer, a colorectal cancer, a gastric cancer, a liver cancer, a lung cancer, an ovarian cancer, a pancreatic cancer, a prostate cancer, a skin cancer, a testicular cancer, a blood cancer, brain cancer, and a vaginal cancer cell. In some embodiments, the blood cancer cell is a leukemia, a non-Hodgkin lymphoma, a Hodgkin lymphoma, or a multiple myeloma cell. In some embodiments leukemia cell is an Acute Myeloid Leukemia (AML) cell.

In some embodiments, the RNA-seq data is obtained by performing sequencing on cells derived from cancer tissue. In some embodiments, the sample cell is derived from a tissue, a blood sample, a cell line, an organoid, saliva, cerebrospinal fluid, or other bodily fluids. In some embodiments, the first cell and the second cell come from the same subject or the first cell and the second cell come from different subjects.

In some embodiments, the method further comprises generating an output for constructing a personalized cancer vaccine from the selected cell surface antigen. In some embodiments, the personalized cancer vaccine comprises at least one peptide sequence or at least one nucleotide sequence encoding the selected cell surface antigen.

In some embodiments, the method further comprises receiving information from a user for example via a computer network comprising a cloud network. In some embodiments, the method further comprises a user interface allowing a user to sort membrane topology values, filter B cell accessibility values, filter T cell antigenicity values, select information stored in the database, merge topology values, accessibility values, and antigenicity values with the selected information stored in the database, select cell surface antigen sequences and cell surface antigen derived peptides, or a combination thereof. In some embodiments, the method comprises a software module allowing the user to sort, filter, or rank the one or more cell surface antigen sequences or cell surface antigen derived peptides based on user-selected criteria. In some embodiments, the method further comprises generating an output for constructing a personalized cancer vaccine from the selected cell surface antigen.

In another aspect, the disclosure provides for methods of treating a subject having a cancer, comprising performing any of the methods above and further comprising obtaining a cancer vaccine comprising the selected cell surface antigen, and administering the cancer vaccine to the subject.

In another aspect, the disclosure provides for methods of treating a subject having a cancer, comprising performing any of the methods above and further comprising generating an antibody, ADC, or CAR-T cell that specifically binds the selected peptide. In some embodiments, the method further comprises obtaining the antibody, ADC, or CAR-T cell that specifically binds the selected peptide, and administering the antibody, ADC, or CAR-T to the subject.

In another aspect, the disclosure provides for methods of treating a subject having a cancer, comprising performing any of the methods above and further comprising generating a TCR engineered T cell that specifically binds the selected peptide. In some embodiments, the method further comprises obtaining the TCR engineered T cell that specifically binds the selected peptide, and administering the TCR engineered T cell to the subject.

In another aspect, the disclosure provides for isolated peptides comprising a cell surface antigen comprising a sequence set forth in TABLE 1, wherein the peptide is no more than 100 amino acids in length, and an optional pharmaceutically acceptable carrier. In some embodiments, the peptide is no more than 30 amino acids in length or 20 amino acids in length. In some embodiments, the amino acid sequence of the peptide consists essentially of or consists of an amino acid sequence set forth in TABLE 1. In some embodiments, the peptide comprises an amino acid sequence set forth in TABLE 1 and is presentable by a major histocompatibility complex (MHC) Class I or MHC Class II. In any of the above compositions the peptide can be synthetic.

In another aspect, the disclosure provides for a recombinant cell engineered to express one or more peptides comprising the amino acid sequences set forth in Table 1 and Table 2.

In another aspect, the disclosure provides a pharmaceutical composition comprising a peptide, e.g., a synthetic peptide, disclosed herein and a pharmaceutically acceptable carrier or excipient. The pharmaceutical composition optionally comprises a plurality of peptides (e.g., 2, 3, 4, 5, 6, 7, 8, 9, 10, or more) disclosed herein and a pharmaceutically acceptable carrier or excipient.

In another aspect, the disclosure provides a pharmaceutical composition comprising a nucleic acid, e.g., a synthetic nucleic acid, encoding the peptide disclosed herein and a pharmaceutically acceptable carrier or excipient. The pharmaceutical composition comprises one or more nucleic acids encoding a plurality of peptides (e.g., 2, 3, 4, 5, 6, 7, 8, 9, 10, or more) disclosed herein and a pharmaceutically acceptable carrier or excipient.

In another aspect, the disclosure provides a vaccine that stimulates a T cell mediated immune response when administered to a subject. The vaccine may comprise any of the above described pharmaceutical compositions. In some embodiments, the vaccine is a priming vaccine and/or a booster vaccine.

In another aspect, the disclosure provides a method for determining whether a subject has cancer, the method comprising detecting the presence and/or amount of (i) one or more peptides disclosed above and/or (ii) T cells reactive with one or more peptides disclosed above, in a sample harvested from the subject thereby to determine whether the subject has cancer. In some embodiments, the method further comprises selecting a treatment regimen based upon the detected presence or amount of peptide. The presence or amount of the peptide may be determined using RNA-seq, anti-peptide Antibodies, mass spectrometry, tetramer assays, or a combination thereof. The presence or amount of the T cells may be determined by a PCR reaction, tetramer assay, Enzyme Linked Immuno Spot Assay (ELISpot), or an Activation Induced Marker (AIM) assay. In some embodiments, the sample is a tissue, a blood sample, a cell line, an organoid, saliva, cerebrospinal fluid, or other bodily fluids harvested from the subject.

In another aspect, the disclosure provides a method for treating a cancer in a subject, the method comprising administering any of the above described pharmaceutical compositions or vaccines to the subject. The cancer can be for example a bone cancer, a breast cancer, a colorectal cancer, a gastric cancer, a liver cancer, a lung cancer, an ovarian cancer, a pancreatic cancer, a prostate cancer, a skin cancer, a testicular cancer, a blood cancer, brain cancer, or a vaginal cancer. In some embodiments the blood cancer is a leukemia, a non-Hodgkin lymphoma, a Hodgkin lymphoma, or a multiple myeloma. In some embodiments, the leukemia is Acute Myeloid Leukemia (AML). In some embodiments, the pharmaceutical composition is administered parenterally or is administered intravenously.

In another aspect, the disclosure provides computer-implemented systems and methods for identifying a disease-specific cell surface antigen or cell surface antigen derived peptide comprising: obtaining a first RNA-seq data set from a first sample cell and a second RNA-seq data set from a second diseased sample cell; assembling full length mRNA transcript sequences and extracting genomic loci coordinates of the mRNA transcript sequences; clustering of full length mRNA transcript sequences encoded at the same genomic loci and extraction of exon duo or exon trio mRNA sequences; selecting the most representative full length mRNA transcript sequences; identifying stable full length mRNAs transcripts; translating, in silico the stable full length mRNA transcripts into protein isoform sequences; identifying protein isoform sequences that are predicted to be stable; determining B cell antibody accessibility of the protein isoform sequences by using an algorithm to classify the polarity, hydrophobicity, and surface accessibility of peptides derived from the protein isoform sequences; determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; generating a first set of antigenic cell surface antigen sequences based on the first RNA-seq data set and a second set of antigenic cell surface antigen sequences based on the second RNA-seq data set ranked by B cell antibody accessibility and T cell antigenicity; and determining unique antigenic cell surface antigen sequences by comparing the first set of antigenic cell surface antigen sequences and the second set of antigenic cell surface antigen sequences and selecting cell surface antigen sequences present in the second set and not the first set; thereby identifying one or more unique cell surface antigen sequences that are disease specific. In some embodiments, the method further comprises determining membrane topologies for each protein isoform sequence and filtering for membrane bound protein isoform sequences. In some embodiments, the diseased sample cell is a cancer cell.

For a fuller understanding of the nature and advantages of the present disclosure, reference should be had to the ensuing detailed description taken in conjunction with the accompanying figures. The present disclosure is capable of modification in various respects without departing from the present disclosure. Accordingly, the figures and description of these embodiments are not restrictive.

### BRIEF DESCRIPTION OF THE DRAWINGS

These and other features, aspects, and advantages of the present invention will become better understood with regard to the following description, and accompanying drawings, where:

FIG. 1A illustrates an overview of the SpliceIO workflow. SpliceImpact™ is a module from SpliceCore. The MB module and the TB module are modules developed for SpliceIO. FIG. 1B depicts a block diagram of the cell surface antigen identification system, in accordance with an embodiment. FIG. 1C shows an exemplary non-limiting schematic diagram of a digital processing device with one or more CPUs, a memory, a communication interface, and a display.

FIG. 2A-FIG. 2C illustrate a scalability comparison between SpliceCore and the popular open-source rMATs. (FIG. 2A) Run time by subsampling (82/1,312 RNA-seq datasets) illustrates the time-cost of recurrently analyzing a large data repository (FIG. 2B) Timing at different sample size and (FIG. 2C) associated memory requirements demonstrates that SpliceCore, but not rMATs can analyze >200 datasets in a single virtual machine. All the RNA-seq data were from the BRCA dataset in TCGA.

FIG. 3A illustrates the predictive performance of SpliceCore (upper curve) outperforms known approaches to predict splicing-mediated protein integrity utilized in other studies (Conservation ROC, Domain ROC, Secondary ROC, tertiary ROC, Multi-Class ROC). FIG. 3B illustrates an unsupervised feature weighting by hierarchical clustering performed on known antigenic and non-antigenic peptide sequences from the Immune Epitope Database (IEDB) to identify features associated with antigenicity.

FIG. 4A illustrates ROC plots showing the performance (AUC) of 5 models trained on antigenic and non-antigenic peptide sequences from the Immune Epitope Database (IEDB). FIG. 4B illustrates variable importance (mean decrease in Gini) was performed for the Random Forest classifier to identify most informative features associated with antigenicity.

FIG. 5 illustrates ROC plots (top) show performance (AUC) of SpliceIO (upper line) vs. the IEDB antigenicity prediction tool (lower line) in classifying a test dataset of 1324 bacterial peptide sequences. Precision (P, bottom) is higher in SpliceIO vs. IEDB for non-antigenic (N) and antigenic (A) peptides, with fewer false positives (recall, R) identified using SpliceIO.

FIG. 6A illustrates ROC plots depict performance (AUC) of a Random Forest classifier trained on surface-bound and intracellular proteins, signal and non-signal peptide regions, or the combined data. FIG. 6B illustrates ROC plots of benchmarking results comparing SpliceIO Type (top line) and SignalP5.0 (lower line) classifiers.

FIG. 7 illustrates training features and mode by classifier.

FIG. 8A illustrates an exemplary data workflow. FIG. 8B Shows the levels of mRNA isoforms for ADGRE5/CD97 by qPCR. Cells are K-562 (leukemia), HCT116 (colon cancer) and U521 (glioblastoma). The asterisk shows AML specificity. FIG. 8C shows a diagram of the predicted protein structure for ADGRE5/CD97. The labeled amino acids are deleted from the short isoform. Predictions were made using Protter (available at URL: wlab.ethz.ch/protter/start/).

FIG. 9A-FIG. 9B illustrate exemplary protein isoforms. The mRNA contains 7 exons, 5 of which are protein coding. FIG. 9A shows the protein isoform expressed in normal cells. FIG. 9B shows the isoform expressed in breast cancer. The inclusion of a novel exon creates an extracellular protein loop containing an antigenic peptide. The novel mRNA has a substantially different open reading frame. The protein isoform shown correspond to the cell surface antigen provided in PEP ID NO: PEP17.

FIG. 10 illustrates an exemplary protein isoform. The left panel shows the protein isoform expressed in normal cells. The right panel shows the isoform expressed in breast cancer. The exclusion of an exon creates a novel peptide, without a substantial part of the normal isoform. The novel mRNA has a substantially different open reading frame.

### DETAILED DESCRIPTION

Various features and aspects of the invention are discussed in more detail below.

The invention is based, in part on the discovery of a method to identify alternative splicing derived cell surface antigens that are invisible to current neoantigen identification methods that rely on whole-exome sequencing (WES) data and are unable to identify these new splicing junctions. New splicing junctions resulting in cell surface antigens are useful in, for example, development of cancer drugs such as Immuno-Oncology applications.

Accordingly, the disclosure provides methods to identify cell surface antigens derived from alternative splicing events, nucleic acids, expression constructs, vectors, and cells comprising the cell surface antigens. The disclosure also provides for methods of making and using a composition useful in the treatment of a subject with a disease characterized by the cell surface antigen, and methods of treatment of a subject with a disease characterized by the cell surface antigen.

Unless otherwise defined herein, scientific and technical terms used in this application shall have the meanings that are commonly understood by those of ordinary skill in the art.

Generally, nomenclature used in connection with, and techniques of, pharmacology, cell and tissue culture, molecular biology, cell and cancer biology, neurobiology, neurochemistry, virology, immunology, microbiology, genetics and protein and nucleic acid chemistry, described herein, are those well-known and commonly used in the art. In case of conflict, the present specification, including definitions, will control.

The practice of the present disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Sambrook and Russell, Molecular Cloning: A Laboratory Manual, 3rd. ed., Cold Spring Harbor Laboratory Press, Cold Spring Harbor, N.Y. (2001); Ausubel et al., Current Protocols in Molecular Biology, John Wiley & Sons, N Y (2002); Harlow and Lane Using Antibodies: A Laboratory Manual, Cold Spring Harbor Laboratory Press, Cold Spring Harbor, N.Y. (1998); Coligan et al., Short Protocols in Protein Science, John Wiley & Sons, N Y (2003); Short Protocols in Molecular Biology (Wiley and Sons, 1999).

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

Throughout this specification and embodiments, the word “comprise,” or variations such as “comprises” or “comprising,” will be understood to imply the inclusion of a stated integer or group of integers but not the exclusion of any other integer or group of integers.

It is understood that wherever embodiments are described herein with the language “comprising,” otherwise analogous embodiments described in terms of “consisting of” and/or “consisting essentially of” are also provided.

The term “including” is used to mean “including but not limited to.” “Including” and “including but not limited to” are used interchangeably.

Any example(s) following the term “e.g.” or “for example” is not meant to be exhaustive or limiting.

Unless otherwise required by context, singular terms shall include pluralities and plural terms shall include the singular.

The articles “a” and “an” are used herein to refer to one or to more than one (i.e., to at least one) of the grammatical object of the article. By way of example, “an element” means one element or more than one element. Reference to “about” a value or parameter herein includes (and describes) embodiments that are directed to that value or parameter per se. For example, description referring to “about X” includes description of “X.” Numeric ranges are inclusive of the numbers defining the range.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in an embodiment of the disclosure.

Exemplary methods and materials are described herein, although methods and materials similar or equivalent to those described herein can also be used in the practice or testing of the present disclosure. The materials, methods, and examples are illustrative only and not intended to be limiting.

### I. Definitions

The following terms, unless otherwise indicated, shall be understood to have the following meanings:

As used herein, “residue” refers to a position in a protein and its associated amino acid identity.

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methylphosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5 ‘ and 3’ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NRi (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

The terms “polypeptide,” “oligopeptide,” “peptide” and “protein” are used interchangeably herein to refer to chains of amino acids of any length. The chain may be linear or branched, it may comprise modified amino acids, and/or may be interrupted by non-amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

The term “sequence similarity,” in all its grammatical forms, refers to the degree of identity or correspondence between nucleic acid or amino acid sequences that may or may not share a common evolutionary origin.

“Percent (%) sequence identity” or “percent (%) identical to” with respect to a reference polypeptide (or nucleotide) sequence is defined as the percentage of amino acid residues (or nucleic acids) in a candidate sequence that are identical with the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

“Homologous,” in all its grammatical forms and spelling variations, refers to the relationship between two proteins that possess a “common evolutionary origin,” including proteins from superfamilies in the same species of organism, as well as homologous proteins from different species of organism. Such proteins (and their encoding nucleic acids) have sequence homology, as reflected by their sequence similarity, whether in terms of percent identity or by the presence of specific residues or motifs and conserved positions. However, in common usage and in the instant application, the term “homologous,” when modified with an adverb such as “highly,” may refer to sequence similarity and may or may not relate to a common evolutionary origin.

As used herein, “isolated molecule” (where the molecule is, for example, a polypeptide, a polynucleotide, or fragment thereof) is a molecule that by virtue of its origin or source of derivation (1) is not associated with one or more naturally associated components that accompany it in its native state, (2) is substantially free of one or more other molecules from the same species (3) is expressed by a cell from a different species, or (4) does not occur in nature.

The term “subject” encompasses a cell, tissue, or organism, human or non-human, whether in vivo, ex vivo, or in vitro, male or female. The term subject is inclusive of mammals including humans.

As used herein, a “vector,” refers to a recombinant plasmid or virus that comprises a nucleic acid to be delivered into a host cell, either in vitro or in vivo. A “recombinant viral vector” refers to a recombinant polynucleotide vector comprising one or more heterologous sequences (i.e., a nucleic acid sequence not of viral origin). In the case of recombinant AAV vectors, the recombinant nucleic acid is flanked by at least one inverted terminal repeat sequence (ITR). In some embodiments, the recombinant nucleic acid is flanked by two ITRs.

As used herein, the term “ORF” means open reading frame.

As used herein, the term “antigen” is a substance that induces an immune response.

As used herein, the term “neoantigen” is an antigen that has at least one alteration that makes it distinct from the corresponding wild-type, parental antigen, e.g., via mutation in a tumor cell or post-translational modification specific to a tumor cell. A neoantigen can include a polypeptide sequence or a nucleotide sequence. A mutation can include a frameshift or nonframeshift indel, missense or nonsense substitution, splice site alteration, genomic rearrangement or gene fusion, or any genomic or expression alteration giving rise to a neoORF. A mutation can also include a splice variant. Post-translational modifications specific to a tumor cell can include aberrant phosphorylation. Post-translational modifications specific to a tumor cell can also include a proteasome-generated spliced antigen.

As used herein, the term “tumor neoantigen” is a neoantigen present in a subject's tumor cell or tissue but not in the subject's corresponding normal cell or tissue.

As used herein, the term “neoantigen-based vaccine” is a vaccine construct based on one or more neoantigens, e.g., a plurality of neoantigens.

As used herein, the term “coding region” is the portion(s) of a gene that encode protein.

As used herein, the term “epitope” is the specific portion of an antigen typically bound by an antibody or T cell receptor.

As used herein, the term “immunogenic” is the ability to elicit an immune response, e.g., via T cells, B cells, or both.

As used herein, the term “alternative splicing” is a mechanism by which different forms of mature mRNAs (messengers RNAs) are transcribed from the same ORF. Alternative splicing is a regulatory mechanism by which variations in the incorporation of the exons, or coding regions, into mRNA leads to the production of more than one related protein, or isoform.

As used herein, “protein isoform” or “isoform” is a member of a set of highly similar proteins that originate from a single gene or gene family and are the result of splicing mRNA transcripts. ORFs mRNA transcripts can comprise introns and exons. While many perform the same or similar biological roles, some isoforms have unique functions. A set of protein isoforms may be formed from alternative splicing, variable promoter usage, or other post-transcriptional modifications of a single gene.

As used herein, the term “cell surface antigen” comprises proteins and peptides that are presented on the surface of a cell. Cell surface antigens can comprise alternatively spliced membrane-bound and MHC presented neoantigens and as well as any membrane bound alternatively spliced protein isoforms accessible to antibodies or T cell receptors. Cell surface antigens can be presented at the cell surface in an MHC dependent or MHC independent way. Typically, MHC dependent peptide presentation is dependent on MHC I or MHC II recognition of short peptides. Membrane bound alternative splicing derived protein isoforms may comprise a transmembrane domain. Their major isoform proteins may or may not comprise a transmembrane domain. Membrane bound alternative splicing derived protein isoforms can comprise neoantigens that may or may not be presented at the cell surface. In some embodiments neoantigens can be derived from membrane bound alternative splicing derived protein isoforms. Thus, a membrane bound alternative splicing derived protein isoforms and their fragments may be presented at the cell surface in two ways (1) as transmembrane protein, and (2) by an MHC after processing by the cellular machinery into a MHC presentable peptide.

“Major histocompatibility complexes” (MHC), also termed Human Leukocyte Antigens (HLA) in humans are glycoproteins expressed on the surface of nucleated cells that act as proteomic scanning chips by providing insight into the status of cellular health. MHCs continuously sample peptides from normal host cellular proteins, cancer cells, inflamed cells and bacterial, viral and parasite infected cells and present short peptides on the surface of cells for recognition by T lymphocytes. Presented peptides can also be derived from proteins that are out of frame or from sequences embedded in the introns, or from proteins whose translation is initiated at codons other than the conventional methionine codon, ATG. There are two classes of MHCs in mice and humans, namely MHC I and MHC II.

The phrase “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio.

The phrase “pharmaceutical composition” refers to a mixture containing a specified amount of a therapeutic, e.g., a therapeutically effective amount, of a therapeutic compound in a pharmaceutically acceptable carrier to be administered to a mammal, e.g., a human, in order to treat a disease.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

Each embodiment described herein may be used individually or in combination with any other embodiment described herein.

### II. SpliceIO

Disclosed herein are systems and methods for identifying alternative splicing derived cell surface antigen sequences. In some embodiments, the systems and methods herein include a platform, e.g., cloud-based platform, to detect, quantify, and analyze cell surface antigens derived from alternative splicing events from user input data such as RNA sequence (RNA-seq) data. Non-limiting examples of input data files includes BAM, SAM, FASTQ, FASTA, BED, and GTF files.

Generally, the cell surface antigen identification system 110 analyzes one or more RNA-seq data sets from one or more sample cells to identify cell surface antigens.

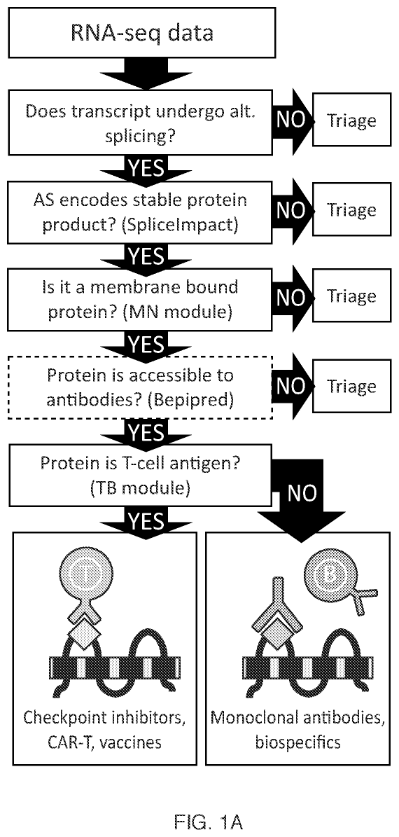

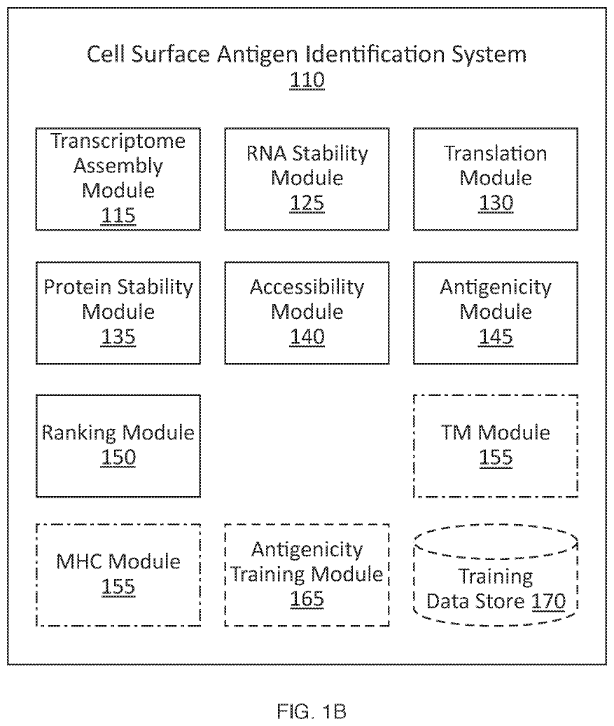

The cell surface antigen identification system 110 can include one or more computers, embodied as a computer system 180 as discussed below with respect to FIG. 1C. Therefore, in various embodiments, the steps described in reference to the cell surface antigen identification system 110 are performed in silico.

In various embodiments, to generate the cell surface antigen identification, the cell surface antigen identification system 110 extracts features from the one or more RNA-seq data sets and applies one or more trained prediction models to analyze the features of the one or more data sets.

Reference is now made to FIG. 1B which depicts a block diagram illustrating the computer logic components of the cell surface antigen identification system 110, in accordance with an embodiment. Here, the cell surface antigen identification system 110 includes a transcriptome assembly module 115, a RNA stability module 125, a translation module 130, a protein stability module 135, an accessibility module 140, an antigenicity module 145, a ranking module 150, a TM module 155, a MHC module 160, an antigenicity training module 165, and a training data store 170. In various embodiments, the cell surface antigen identification system 110 can be configured differently with additional or fewer modules. As another example, the cell surface antigen identification system 110 need not include the TM module 155, the MHC module 160, the antigenicity training module 165, or the training data store 170 (as indicated by their dotted lines in FIG. 1B), and instead, the TM module 155, the MHC Module 160, the antigenicity training module 165, or the training data store 170 are employed by a different system and/or party.

Generally, the transcriptome assembly module 115 builds full length mRNA transcript sequences from RNA-seq data sets captured from sample cells. The transcriptome assembly module 115 clusters mRNA transcript sequences mapping to the same genomic loci to generate transcript sequence blocks from which exon duo and exon trio RNA sequences are extracted. The most representative mRNA transcript sequence is selected to determine the full length protein. The most representative mRNA transcript sequence for the long and short isoform is selected based on criteria such as whether the transcript is annotated as the principal isoform in Appris, (apprisws.bioinfo.cnio.es/landing_page/) or is labeled with the highest Appris score, or has the longest protein sequence. The representative mRNA transcript sequence for the opposite isoform is selected based on criteria such as whether the mRNA transcript produces an identical protein sequence, or shares the maximum number of exons or identical splice sites.

The RNA stability module 125 assesses the stability of the mRNA transcripts. This is important since mRNA can be degraded by nonsense mediated decay (NMD) before the mRNA can be translated into proteins and peptides. In various embodiments, the RNA stability module 125 provides data in the form of stable full length mRNA transcripts to the RNA translation module 130 for translation of the mRNA transcripts into protein isoform sequences.

The translation module 130 translates the stable full length mRNA transcripts into protein isoform sequences. In various embodiments, the translation module 130 provides data in the form of protein isoform sequences to the protein stability module 135 for protein isoform stability assessment.

The protein stability module 135 determines protein isoform stability. In various embodiments, the protein stability module 135 provides data in the form of stable protein isoform sequences to the accessibility module 140 for determination of B cell accessibility, the antigenicity module 145 for determination of T cell antigenicity, or the TM module 155 for determination of transmembrane topology.

The accessibility module 140 determines B cell accessibility of stable protein isoform sequences by classifying the polarity, hydrophobicity, and surface accessibility of peptide sequences derived from the stable protein isoform sequences. In various embodiments, the accessibility module 140 provides data in the form of rankings for polarity, hydrophobicity, and surface accessibility of the stable protein isoform sequences to the ranking module 150 for ranking and classification of the stable protein isoform sequences.

The antigenicity module 145 determines T cell antigenicity of stable protein isoform sequences by using a machine learning algorithm. various embodiments, the antigenicity module 145 provides stable protein isoform sequences that are classification for two characteristics (0 responsive or non-responsive, and/or (ii) antigenic or non-antigenic to the ranking module 150 for ranking and classification of the stable protein isoform sequences.

The machine learning algorithm of the antigenicity module 145 can be trained with the antigenicity training module 165 using training data stored in the training data store 170. The antigenicity module 145 classifies stable protein isoform sequences into two characteristics (i) responsive or non-responsive, and/or (ii) antigenic or non-antigenic. As an example, the antigenicity training module 165 and training data store 170 are employed by a different system and/or party.

The TM module 155 determines transmembrane topology of the stable protein isoform sequences. In various embodiments, the TM module 155 provides stable protein isoform sequences that comprise transmembrane domains to the ranking module 150 for ranking and classification of the stable protein isoform sequences.

The MHC module 160 determines MHC I or MHC II binding of the stable protein isoform sequences. In various embodiments, the MHC module 160 provides stable protein isoform sequences that bind MHC I or MHC II complexes to the ranking module 150 for ranking and classification of the stable protein isoform sequences.

The ranking module 150 compares and ranks the stable protein isoform sequences identified for a first cell sample and a second cell sample. Stable protein isoform sequences that are unique for a cell sample are ranked according to the output by the accessibility module 140, antigenicity module 145, TM module 155, and MHC module 160.

In various embodiments, the ranking module ranks the predicted scores of the outputs of the accessibility module 140 and the antigenicity module 145 compared to reference scores. In various embodiments, the ranking module ranks the predicted scores of the outputs of the accessibility module 140, antigenicity module 145, TM module 155, and MHC module 160 compared to reference scores. In various embodiments, the one or more reference scores have threshold cutoff values. For example, a threshold cutoff value can be between 0 and 1, such as 0.1, 0.2, 0.3, 0.4, 0.5, 0.6, 0.7, 0.8, or 0.9. In particular embodiments, a threshold value is 0.1. In particular embodiments, a threshold value is 0.5. Therefore, if the predicted score is above the threshold reference score, the cell surface antigen is classified into one category (e.g., antigenic, B cell antibody accessible, membrane bound). If the predicted score is below the threshold reference score, the cell surface antigen is classified into a different category (e.g., not antigenic, not B cell antibody accessible, not membrane bound develop).

Provided herein is an exemplary platform known as “SpliceIO.” In some embodiments, the SpliceIO platform is equivalent to the compute back end core. In some embodiments, the SpliceIO platform may include one or more modules selected from: SpliceImpact™, SpliceTrap™, and two main Machine Learning (ML) modules: an “immunoncology” (TO) module to predict protein antigenicity and a “membrane bound” (MB) module, to predict protein topology and membrane localization. Additionally or alternatively SpliceIO comprises a membrane topology prediction module for example Phobius phobius.sbc.su.se/), a sequential B-Cell Epitope Predictor for example BepiPred2.0 (www.cbs.dtu.dk/services/BepiPred/), and a peptide/MHC binding predictor for example NetMHCpan 4.1 (www.cbs.dtu.dk/services/NetMHCpan/). An exemplary SpliceIO workflow is illustrated in FIG. 1.

In some embodiments, the SpliceIO platform includes one or more of: a software module, an application, an algorithm, a user interface, a memory, a digital processing device, a data storage, a database, a cluster of computing notes, a cloud network, a communications element, and a computer program.

In some embodiments, the SpliceIO platform may take as its input user-provided datasets including, but not limited to, RNA-seq data. In some embodiments, RNA-seq data can be derived from sequencing a single cell (single-cell RNA sequencing, scRNA-seq) or from sequencing bulk cells. In some embodiments, the single cell or the bulk cells can be from a tissue sample, a blood sample, a cell line sample, an organoid sample, saliva sample, cerebrospinal fluid sample, or other bodily fluid sample. In some embodiments, the cells are from a normal tissue sample or a diseased tissue sample.

In some embodiments, the systems and methods herein include a software module allowing the user to sort, filter, merge the plurality of cell surface antigen values representing the AS changes with the information stored in the database, or a combination thereof. This functionality may allow users to rank and prioritize the most important AS changes detected with SpliceIO modules, according to criteria of their choice.

In some embodiments, the systems and methods herein are configured to use cloud computing, which can advantageously enable parallel distributed computing, cluster computing, compute scalability, training on larger datasets, integration of various data types, and perform deeper search for novel splicing events in reasonable time with lower cost. The alternative to the cloudbased platform herein is to maintain a physical supercomputer. There can be tremendous costs associated with maintaining, protecting and updating such resources. Another benefit of cloud computing can be its scalability. Large cloud computing resources can be temporarily built, utilized, and discarded so that the computing costs vary in direct relation to demand.

### SpliceTrap™

In some cases, the systems and methods herein include a SpliceTrap™ module. The SpliceTrap module can include a probability model, e.g., Bayesian model, for the quantification of AS. Using the front end, or equivalently, the user interface, the user can select which data file(s), e.g., FASTA/FASTQ, the user wants to upload for analysis by the SpliceTrap™ module. This upload can create an entry in the SpliceTrap™ queue which may trigger the creation of the SpliceTrap™ cluster. If there is a cluster currently created, a run can be queued. The SpliceTrap™ pipeline can then process the data and produce its output. After SpliceTrap™ completes running, the output may be created and uploaded to the user's SpliceTrap™ results database. The SpliceTrap™ module can analyze pair-end or single-end transcriptome(s) or genome(s) data for any species for which a TXdb reference can be produced.

In some embodiments, a cluster may include one or more digital processing devices herein, or equivalently, computing nodes. The digital processing devices may or may not be remotely located from the systems and methods herein. In some cases, the devices or computing nodes of the cluster communicate with others in the cluster or the systems and methods herein via a computer network, e.g., a cloud network.

The SpliceTrap™ module herein, in some cases, includes a software module mapping at least a portion of the user-input information to a database. In some cases, the information comprises biological data related to genome(s), transcriptome(s), or both and/or biological data that can be mapped to genome(s), transcriptome(s), or both. The SpliceTrap™ module may further include a software module computing a set of data-dependent parameters from the mapped information. In some cases, the SpliceTrap™ module is configured to perform heuristic approximation to estimate the set of data-dependent parameters. In some cases, the data dependent parameters from TXdb mapped reads include, but are not limited to, one or more of: fragment size distribution, fragment size distribution model and its parameters, inclusion ratio distribution, inclusion ratio distribution model and its parameters, length of an exon duo or trio isoform, and expression level of an exon duo or trio isoform. The heuristic approximation can result in a significantly decreased runtime than a runtime to compute an exact optimization of the data-dependent parameters.

### TXdb Database

The TXdb database herein can include a customized database which incorporates at least 7 million splicing events derived from the analysis of public RNA-seq datasets, for example including >10.000 from TCGA with ˜1.500 BRCA breast cancer tissues, and from the Genotype-tissue expression repository (GTEx) with 3.000 normal breast tissues. Splicing events are defined as any combination of 2 or 3 exons in the transcriptome (i.e., exon duos or exon trios, described in Wu J. et al., Bioinformatics. (2011) (21):3010-6). Every exon duo or exon trio is represented by two “inclusion” splice junctions and one “skipping” splice junction. TXdb creates a search space for novel junction discovery useful to differentiate self from non-self splice junctions. The size of this customized database can be bigger (about 10 times or more) than comparable open source databases. In some cases, the TXdb database includes a database configured to allow interrogation through RNA-seq data mapping, wherein each entry of the database may comprise an independent splicing event that is configured to be analyzed for example by the SpliceTrap™ module.

### SpliceImpact™

The systems and methods herein include a SpliceImpact™ module. The SpliceImpact™ module includes a statistical method that integrates protein-protein interactions, RNA and protein structure, genetic variation, genetic conservation, disease pathways data and custom disease-specific features derived from any public or proprietary biological data source, to prioritize biologically relevant AS changes that can potentially cause disease. In some cases, the SpliceImpact™ module can include one or more steps selected from: estimating the probability of AS events to down-regulate protein function through nonsense mediate decay (NMD); estimate probability of AS events of damaging protein structures through protein domain deletion; estimating mutability of AS events (the mutability can be determined as the proportion of nucleotides in an exon that when mutated, cause a damaging effect on protein function); mapping AS events with their respective scores in a pathway-pathway network; and outputting list of AS ranked by biological relevance. The protein domains can be retrieved from InterPro database or predicted de-nova using Interpro scan, Pfam, Coils, Prosite, CDD, TIGRFAM, SFLD, SUPERFAMILY, Gene3d, SMART, PRINTS, PIRASF, PRoDom,MobiDBLite, TMHMM and other algorithms to predict functional and structural elements based on primary protein sequences. To estimate the damaging potential of single nucleotide variants (SNV), a combination of functional predictive methods (e.g., SIFT, PolyPhen, Mutation Tester, Mutation assessor, LRT and FATHMM) can be used. Additive damaging score of one or more nucleotides in an exon can be used to prioritize damaging AS events.

In some cases, the systems and methods herein include a software module processing the plurality of AS values with information stored in the database or a second database to identify a plurality of prioritized biologically or clinically relevant AS changes, wherein the software module processing the plurality of AS values with information stored in the database or a second database comprises a supervised or semi-supervised machine learning algorithm, and wherein the information comprises metadata obtained from annotations of a plurality of classes of AS based on public RNA-seq data, CLIP-seq data, genomic data, script data, other biological data or calculated de novo based on DNA, RNA or protein sequences using proprietary or open-source algorithms. In some cases, the systems and methods herein include a software module generating the annotations, wherein the annotation comprises information related to public RNA-seq data and metadata. In some cases, the annotations can also provide mapping reference for the user's input information. In some cases, the systems and methods herein include a software module performing a semi-supervised or supervised machine learning algorithm, wherein the machine learning algorithm takes the plurality of features as an input and outputs a predictive algorithm and/or prediction of impact of AS events on protein structures, protein functions, RNA stability, RNA integrity, or biological pathways.

In some cases, the systems and methods herein include a software module processing the plurality of AS values with information stored in a database using the predictive algorithm, prediction (e.g., prediction generated using the predictive algorithm(s) herein or prediction generated using tools external to the systems and methods disclosed herein), and/or the information comprising metadata obtained from annotation of a plurality of classes of AS based on public RNA-seq data. In some cases, the systems and methods herein include a software module generating a plurality of prioritized, and biologically or clinically relevant AS changes based on the plurality of AS values.

The SpliceImpact™ module herein use machine learning classifier/algorithm to integrate larger set of predictive features. Nonlimiting examples of such machine learning classifier/algorithm includes SVM, random forest, neural networks, logistic regression, and deep learning. In some embodiments, the machine learning algorithm is supervised or semi-supervised to leverage the vast amount of unlabeled AS changes for which no conclusive evidence of functional outcome is known. In some cases, the positive training samples include a number of minor human AS changes supported by at least two peptides in PeptideAtlas and not labeled “principal isoform” in the APPRIS database and/or splicing isoforms annotated in Swissprot/ENSEMBL database and supported to result in viable minor splicing events (i.e., low frequency splicing events) as confirmed by TXdb metadata. The positive training set may be separated in two groups of isoforms: minor “skipping” and minor “inclusion” isoforms, and can be used for training separately.

In some embodiments, the SpliceImpact™ module was trained using a gradient boosting classifier on over 45,000 splicing events from the AS database, TXdb, which were labelled as “stable” or “unstable.” 1,027 AS events were labelled as “stable” based on encoding for “minor” splicing isoforms. In some embodiments, the SpliceImpact™ module outputs a score from 0-1, with 1 being highly likely to have an impact on protein structure and function, and 0 having low impact on protein structure and function. In some embodiments, the SpliceImpact™ module also outputs whether mRNA is predicted to enter NMD with “yes” or “no”.

### Membrane Bound (MB) Module

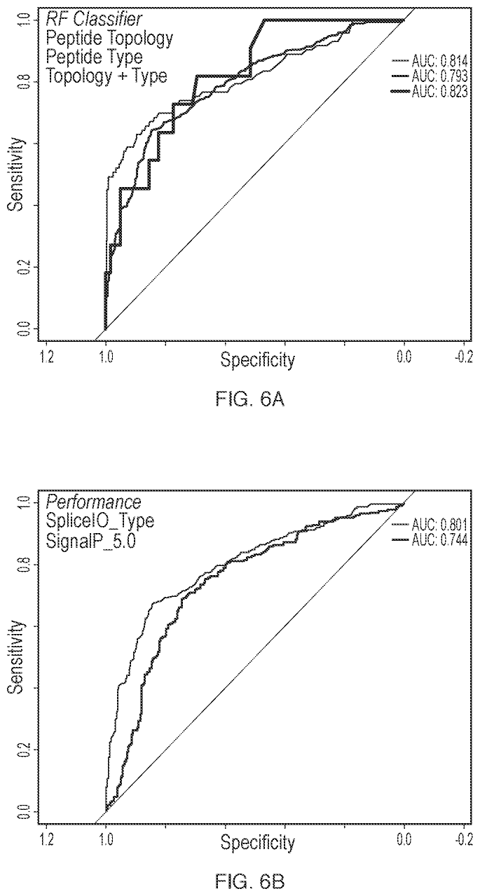

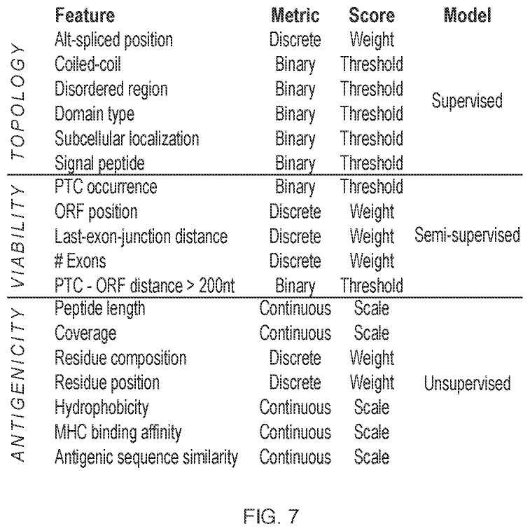

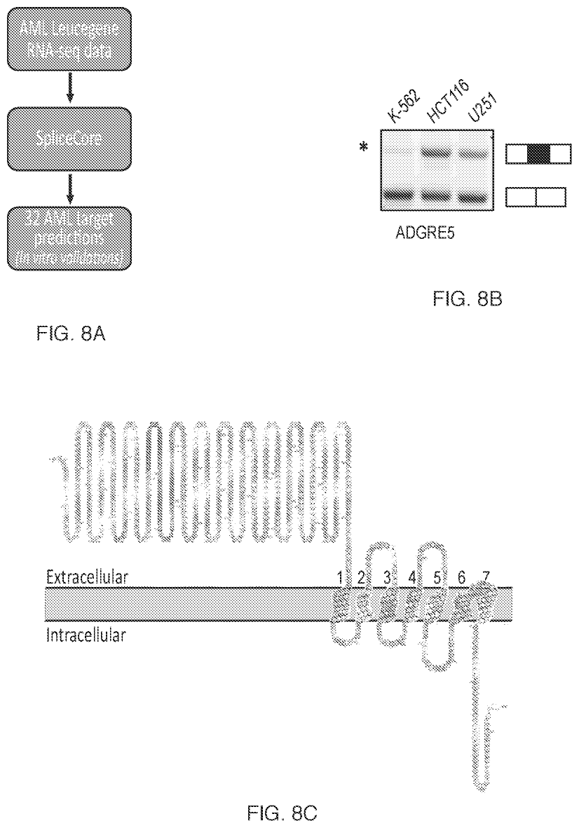

The systems and methods herein include a MB module. The MB module predicts the likelihood of protein isoform to be located on the cell membrane. An exemplary MB module is a machine learning algorithm trained on a dataset of 2,650 protein isoform sequences, which were previously labelled with two characteristics. The first were labelled either “membrane-bound” or “intracellular”, and the second label was either “with” or “without” signal peptides. An exemplary ML learning algorithm is random forest including a grid search with 5-fold cross-validation. As a result, the MB module AUC was 0.79-0.82 using either or both labels (FIG. 6A). In performance assessments, the MB module showed equivalent and/or better sensitivity and specificity when compared to Signal P5.0 (www.cbs.dtu.dk/services/SignalP/), another topology prediction tool, (FIG. 6B). Since random forest assigns probability scores to each protein isoform separately, protein isoform sequences can be scored separately for membrane topology. Another exemplary MB module is the membrane topology prediction module Phobius (phobius.sbc.su.se). The MB module scores the translated isoform protein sequences for transmembrane domains. In some embodiments, the MB module filters the list of protein sequences likely to encode for cell surface proteins based on a list of known genes that encode cell surface proteins. In some embodiments the protein sequences are further filtered using Phobius, which splits the protein sequences into regions based on their relation to the plasma membrane and assigns a topology to each region (cytoplasmic, transmembrane, extracellular, signal peptide).

### T Cell/B Cell (TB) Module

The systems and methods herein include a TB module. The TB module predicts the likelihood of a protein isoform to be accessible to antibodies and the likelihood that the protein isoform will elicit a T cell immune response. Cell surface antigens predicted as “accessible to antibodies” can be targeted with bispecific or monoclonal antibodies. Cell surface antigens further predicted as “antigenic” can be targeted with T-cell based therapeutics such as checkpoint inhibitors, CAR-T, and vaccines. Accordingly, cell surface antigens can be classified as “B” if accessible to antibodies and “T” if they are also predicted to elicit a T cell immune response. The T-cell/B-cell (TB) module takes as input antibody-accessible protein peptides pre-selected using BepiPred2.0 (www.cbs.dtu.dk/services/BepiPred/), to predict their probability to elicit a T-cell immune response. BepiPred2.0 analyses the polarity, hydrophobicity, and surface accessibility of antigenic candidates to identify antibody-accessible protein sequences. BepiPred2.0 outputs an B cell epitope prediction score for each amino acid in a protein sequence. Predicted B cell epitopes are output as peptide sequences, which are generated from consecutive amino acids scoring usually above 0.5. In some embodiments the score can be below 0.5, such as 0.4. The average score is generated for each peptide, then the predicted B cell epitopes are further categorized/filtered for peptide length and % similarity in order to identify sequences that are unique from the other protein isoform's predicted epitopes, as well as from the entire protein sequence of the other protein isoform.

To predict T-cell antigenicity, a ML algorithm trained on known antigenic peptides derived from virus and bacteria in a database of 6751 viral and 4387 non-antigens, and 1324 bacterial peptide sequences, comprising 576 antigens and 748 non-antigens was compiled. The antigenic potential of all viral and bacterial peptide sequences had been previously assessed in vitro by cytokine secretion and cytotoxicity, or in vivo by protection from infection. Peptides in the database that elicit an immune response are classified as “responsive.” In some embodiments the antigenicity module 145 outputs a score from 0-1, with 1 being highly antigenic and 0 having low antigenicity.

In certain embodiments the training peptide sequences comprise peptide sequences having lengths from 5 to 25 amino acids. In certain embodiments the peptide sequences comprise peptide sequences having lengths from 8 to 15 amino acids.

In some embodiments, peptide/MHC binding is also predicted. An exemplary predictor is NetMHCpan 4.1 (www.cbs.dtu.dk/services/NetMHCpan/). The NetMHCpan-4.1 server predicts binding of peptides to any MHC molecule of known sequence using artificial neural networks (ANNs).

The machine learning algorithms can comprise a random forest model, a Bayesian model, a regression model, a neural network, a classification tree, a regression tree, a discriminant analysis, a k-nearest neighbors method, a naive Bayes classifier, support vector machines (SVM), a generative model, a low-density separation method, a graph-based method, a heuristic approach, or a combination thereof.

In some embodiments, the machine learning algorithms herein output algorithm(s) for functional prediction of AS events. The output algorithm(s) may or may not have an explicit or a hidden mathematical expression. The output algorithm(s) may include one or more parameter(s) that can be learned or trained using the machine learning algorithms.

In order to output the algorithm for functional prediction of AS events, a machine learning classifier may include learning the training data, or similarly, a model, or function. For learning, the machine learning algorithm can take training data and/or label as its input data. Learning may be completed when one or more stopping criteria have been reached. For example, a linear regression model having a formula Y=CO+Clxl+C2x2 has two predictor variables, xl and x2, and coefficients or parameters, CO, Cl, and C2. The predicted variable in this example is Y. After the parameters of the model are learned using a machine learning algorithms, values can be entered for each predictor variable in the learned model to generate a result for the dependent or predicted variable (e.g., Y).

A machine learning algorithm herein may use a supervised learning approach. In supervised learning, the algorithm can generate a function or model from training data. The training data can be labeled. The training data may include metadata associated therewith. Each training example of the training data may be a pair consisting of at least an input object and a desired output value. A learning algorithm may require the user to determine one or more control parameters. These parameters can be adjusted by optimizing performance on a subset, for example a validation set, of the training data. After parameter adjustment and learning, the performance of the resulting function/model can be measured on a test set that may be separate from the training set. Regression methods can be used in supervised learning approaches.

A machine learning algorithm may use a semi-supervised learning approach. Semi-supervised learning can combine both labeled and unlabeled data to generate an appropriate function or classifier.

In some embodiments, a machine learning algorithm is interchangeable with a machine learning classifier herein.

The machine learning algorithms can be trained using for example a training data set comprising training protein sequences encoded with two characteristics i) transmembrane or globular or ii) with signal peptide or without signal peptide.

Alternatively or additionally, the machine learning algorithm can be trained using a training data set comprising training peptide sequences encoded with two characteristics (i) responsive or non-responsive and (ii) antigenic or non-antigenic. Training data can be derived by sequencing de-novo from cells, or for example can be derived from publicly available repositories such as TCGA (www.cancer.gov/about-nci/organization/ccg/research/structural-genomics/tcga) and GTEx (gtexportal.org/home/). The training data set may be generated by comparing the set of training protein sequences via alignment to a database comprising a set of known protein sequences. The training data set may be generated based on performing or having performed RNA-seq on a cell line, patient derived line, or cell derived from a healthy donor. The sequencing data can include at least one nucleotide sequence including an alteration. The training data set may be generated based on obtaining RNA-seq data from normal tissue samples. The training data set may be generated based on obtaining RNA-seq data from diseased tissue samples. The training data set may further include data associated with proteome sequences associated with the samples.

### User Interface

In some cases, the systems and methods herein include a user interface core. The user interface core may include a three-tier scheme: (1) project dashboard/screen, user access management and data upload followed by SpliceIO analysis; (2) experiment dashboard/screen, where users can select various SpliceIO outputs to perform case/control comparison; and (3) predictive analytic dashboard/screen where users can combine their proprietary data with TXdb metadata or cell specific data and machine learning precalculated predictions for identification of membrane topology or antigenicity of cell surface antigens.

In some cases, the user interface core herein allows a user to use a user-friendly interface for uploading data for quantification/analysis. Such data may include any biological data. Such data may include RNA-seq data that can be mapped on pre-processed RNA-seq data. Nonlimiting exemplary biological data is raw RNA-seq data. Using the user interface, users can interactively utilize/edit various functionalities of SpliceIO module. For example, after completing a SpliceIO run the user can create sort membrane topology values, filter B cell accessibility values, filter T cell antigenicity values, select information stored in the database, merge topology values, accessibility values, and antigenicity values with the selected information stored in the database, and select cell surface antigens and cell surface antigen derived peptides. The user project owner may access the projects, datasets, and experiments of the project(s), while the project team member may only access specified datasets and/or experiments of the project(s). The administrator may not only access the users' project information but also account information, and/or information of the system and methods herein that is not provided to the users, for example, the parameters and setting of the SpliceIO module.

In some cases, the user interface comprises two or more user environments. For example, the user interface can comprise four different environments of the user interface. The first user environment can be a Project Dashboard wherein the client's projects can be displayed. Project information can include, but is not limited to, the number of RNA-seq datasets analyzed in the project, the run status of the experiments, as well as admitted users and administrators. The second user environment can include Datasets and Experiments. Once RNA-seq datasets are uploaded, they can be analyzed with SpliceIO. The dashboard can show the analysis process and a link to download data processed by SpliceIO. The third user environment can show an Experiments Results interface wherein a table of statistically significant cell surface antigens resulting from alternative splicing events displayed to the user. The fourth user environment can be a membrane topology and antigenicity report for the user wherein the user can filter interesting cell surface antigen candidates. For each candidate, a series of graphics describing the splicing event can be populated to include such data as splicing levels, read coverage, RNA-seq mapping profiles on the genome, information about disease involvement, tissue specificity, transmembrane topology, B-cell antibody accessibility, T cell antigenicity, or MHC binding predictions.

In certain embodiments, the method further comprises receiving information from a user. For example, the information from a user can be received via a computer network comprising a cloud network. In certain embodiments, the method further comprises a software module comprising a user interface allowing a user to sort membrane topology values, filter B cell accessibility values, filter T cell antigenicity values, select information stored in the database, merge topology values, accessibility values, and antigenicity values with the selected information stored in the database, select cell surface antigens and cell surface antigen derived peptides, or a combination thereof. The software module can allow the user to sort, filter, or rank the one or more cell surface antigen or cell surface antigen derived peptides based on user-selected criteria. Additionally or alternatively the method can generate an output for constructing a personalized cancer vaccine from the selected one or more cell surface antigens or peptides. In some embodiments, the personalized cancer vaccine comprises at least one cell surface antigen sequence or peptide sequence or at least one nucleotide sequence encoding the selected cell surface antigen or peptide.

### Digital Processing Device

In some embodiments, the platforms, systems, media, and methods described herein include a digital processing device, or use of the same. In further embodiments, the digital processing device includes one or more hardware central processing units (CPUs) or general purpose graphics processing units (GPGPUs) that carry out the device's functions. In still further embodiments, the digital processing device further comprises an operating system configured to perform executable instructions. In some embodiments, the digital processing device is optionally connected to a computer network. In further embodiments, the digital processing device is optionally connected to the Internet such that it accesses the World Wide Web. In still further embodiments, the digital processing device is optionally connected to a cloud computing infrastructure. In other embodiments, the digital processing device is optionally connected to an intranet. In other embodiments, the digital processing device is optionally connected to a data storage device.

In accordance with the description herein, suitable digital processing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art.

In some embodiments, the digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS x®, UNIX®, and UNIXlike operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry os®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3R, Sony® PS4®, Microsoft® Xbox 360®, Microsoft Xbox One, Nintendo® Wii®, Nintendo® Wii u®, and Ouya®.

In some embodiments, the device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing based storage. In further embodiments, the storage and/or memory device is a combination of devices such as those disclosed herein.

In some embodiments, the digital processing device includes a display to send visual information to a user. In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In yet other embodiments, the display is a headmounted display in communication with the digital processing device, such as a VR headset. In further embodiments, suitable VR headsets include, by way of non-limiting examples, HTC Vive, Oculus Rift, Samsung Gear VR, Microsoft HoloLens, Razer OSVR, FOYE VR, Zeiss VR One, Avegant Glyph, Freefly VR headset, and the like. In still further embodiments, the display is a combination of devices such as those disclosed herein.

In some embodiments, the digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera or other sensor to capture motion or visual input. In further embodiments, the input device is a Kinect, Leap Motion, or the like. In still further embodiments, the input device is a combination of devices such as those disclosed herein.

Referring to FIG. 1C, in a particular embodiment, an exemplary digital processing device 190 is programmed or otherwise configured to perform cell surface antigen sequence identification. The device 180 can regulate various aspects of the present disclosure. In this embodiment, the digital processing device 180 includes a central processing unit (CPU, also “processor” and “computer processor” herein) 190, which can be a single core or multi core processor, or a plurality of processors for parallel processing. The digital processing device 180 also includes memory or memory location 200 (e.g., random access memory, read-only memory, flash memory), electronic storage unit 210 (e.g., hard disk), and communication interface 220 (e.g., network adapter, network interface) for communicating with one or more other systems, and peripheral devices, such as cache, other memory, data storage and/or electronic display adapters. The peripheral devices can include storage device(s) or storage medium 265 which communicate with the rest of the device via a storage interface 270. The memory 200, storage unit 210, interface 220 and peripheral devices are in communication with the CPU 190 through a communication bus 225, such as a motherboard. The storage unit 210 can be a data storage unit (or data repository) for storing data. The digital processing device 180 can be operatively coupled to a computer network (“network”) 230 with the aid of the communication interface 220. The network 230 can be the Internet, an internet and/or extranet, or an intranet and/or extranet that is in communication with the Internet. The network 230 in some cases is a telecommunication and/or data network. The network 230 can include one or more computer servers, which can enable distributed computing, such as cloud computing. The network 230, in some cases with the aid of the device 180, can implement a peer-to-peer network, which may enable devices coupled to the device 180 to behave as a client or a server.

Continuing to refer to FIG. 1C, the digital processing device 180 includes input device(s) 245 to receive information from a user, the input device(s) in communication with other elements of the device via an input interface 250. The digital processing device 180 can include output device(s) 255 that communicates to other elements of the device via an output interface 260.

Continuing to refer to FIG. 1C, the memory 200 may include various components (e.g., machine readable media) including, but not limited to, a random access memory component e.g., RAM) (e.g., a static RAM “SRAM”, a dynamic RAM “DRAM, etc.), or a read-only component (e.g., ROM). The memory 200 can also include a basic input/output system (BIOS), including basic routines that help to transfer information between elements within the digital processing device, such as during device start-up, may be stored in the memory 200.

Continuing to refer to FIG. 1C, the CPU 190 can execute a sequence of machine readable instructions, which can be embodied in a program or software. The instructions may be stored in a memory location, such as the memory 200. The instructions can be directed to the CPU 190, which can subsequently program or otherwise configure the CPU 190 to implement methods of the present disclosure. Examples of operations performed by the CPU 190 can include fetch, decode, execute, and write back. The CPU 190 can be part of a circuit, such as an integrated circuit. One or more other components of the device 190 can be included in the circuit. In some cases, the circuit is an application specific integrated circuit (ASIC) or a field programmable gate array (FPGA).

Continuing to refer to FIG. 1C, the storage unit 210 can store files, such as drivers, libraries and saved programs. The storage unit 210 can store user data, e.g., user preferences and user programs. The digital processing device 180 in some cases can include one or more additional data storage units that are external, such as located on a remote server that is in communication through an intranet or the Internet. The storage unit 210 can also be used to store operating system, application programs, and the like. Optionally, storage unit 210 may be removably interfaced with the digital processing device (e.g., via an external port connector (not shown)) and/or via a storage unit interface. Software may reside, completely or partially, within a computer-readable storage medium within or outside of the storage unit 210. In another example, software may reside, completely or partially, within processor(s) 190.

Continuing to refer to FIG. 1C, the digital processing device 180 can communicate with one or more remote computer systems 280 through the network 230. For instance, the device 190 can communicate with a remote computer system of a user. Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants.

Continuing to refer to FIG. 1C, information and data can be displayed to a user through a display 235. The display is connected to the bus 225 via an interface 240, and transport of data between the display other elements of the device 180 can be controlled via the interface 240.

Methods as described herein can be implemented by way of machine (e.g., computer processor) executable code stored on an electronic storage location of the digital processing device 180, such as, for example, on the memory 200 or electronic storage unit 210. The machine executable or machine readable code can be provided in the form of software. During use, the code can be executed by the processor 190. In some cases, the code can be retrieved from the storage unit 210 and stored on the memory 200 for ready access by the processor 190. In some situations, the electronic storage unit 210 can be precluded, and machine executable instructions are stored on memory 200.

### Non-Transitory Computer Readable Storage Medium

In some embodiments, the platforms, systems, media, and methods disclosed herein include one or more non-transitory computer readable storage media encoded with a program including instructions executable by the operating system of an optionally networked digital processing device. In further embodiments, a computer readable storage medium is a tangible component of a digital processing device. In still further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or nontransitorily encoded on the media.

### Computer Program

In some embodiments, the platforms, systems, media, and methods disclosed herein include at least one computer program, or use of the same. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™ JavaServer Pages (JSP), Hypertext Preprocessor (PHP), Python™, Ruby, Tel, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some embodiments, an application provision system comprises one or more databases accessed by a relational database management system (RDBMS). Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs (such as Java servers, .NET servers, PHP servers, and the like) and one or more web servers (such as Apache, IIS, GWS and the like). The web server(s) optionally expose one or more web services via app application programming interfaces (APis). Via a network, such as the Internet, the system provides browser-based and/or mobile native user interfaces.

In some embodiments, an application provision system alternatively has a distributed, cloud-based architecture and comprises elastically load balanced, auto-scaling web server resources and application server resources as well synchronously replicated databases.

### Mobile Application

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C #, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB NET, or combinations thereof. Compilation is often performed, at least in part, to create an executable program. In some embodiments, a computer program includes one or more executable compiled applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®.

In view of the disclosure provided herein, those of skill in the art will recognize that several plug-in frameworks are available that enable development of plug-ins in various programming languages, including, by way of non-limiting examples, C++, Delphi, Java™, NAP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of nonlimiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### III. Applications

### Identification of Cell Surface Antigens

In some embodiments, the platforms, systems, and methods disclosed herein are applied to medical applications. In one aspect, the proceeding disclosure can be used to identify a cell surface antigen associated with an alternative splicing event in a cell.

As an example, one such method may comprise the steps of (a) obtaining a first RNA-seq data set from a first sample cell and a second RNA-seq data set from a second sample cell; (b) assembling full length mRNA transcript sequences and extracting genomic loci coordinates of the mRNA transcript sequences; (c) clustering of full length mRNA transcript sequences encoded at the same genomic loci and extraction of exon duo or exon trio mRNA sequences; (d) selecting the most representative full length mRNA transcript sequences; (e) identifying stable full length mRNAs transcripts; (f) translating, in silico the stable full length mRNA transcripts into protein isoform sequences; (g) identifying protein isoform sequences that are predicted to be stable; (h) determining B cell antibody accessibility of the protein isoform sequences by using an algorithm to classify the polarity, hydrophobicity, and surface accessibility of peptides derived from the protein isoform sequences; (i) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; (j) generating a first set of antigenic cell surface antigen sequences based on the first RNA-seq data set and a second set of antigenic cell surface antigen sequences based on the second RNA-seq data set ranked by B cell antibody accessibility and T cell antigenicity; and (k) determining unique antigenic cell surface antigen sequences by comparing the first set of antigenic cell surface antigen sequences and the second set of antigenic cell surface antigen sequences and selecting cell surface antigen sequences present in one set and not the other set; thereby selecting one or more unique cell surface antigen sequences.

in certain embodiments the method can comprise identifying one or more cell surface antigens resulting from alternative splicing in a cell comprising the steps of: (a) obtaining a first RNA-seq data set from a first sample cell and a second RNA-seq data set from a second sample cell; (b) assembling full length mRNA transcript sequences and extracting genomic loci coordinates of the mRNA transcript sequences; (c) clustering of full length mRNA transcript sequences encoded at the same genomic loci and extraction of exon duo or exon trio mRNA sequences; (d) selecting the most representative full length mRNA transcript sequences; (e) identifying stable full length mRNAs transcripts; (f) translating, in silico the stable full length mRNA transcripts into protein isoform sequences; (g) identifying protein isoform sequences that are predicted to be stable; (h) determining membrane topologies for each protein isoform; (i) filtering for membrane bound protein isoform sequences; (j) determining B cell antibody accessibility of the protein isoform sequences by using an algorithm to classify the polarity, hydrophobicity, and surface accessibility of peptides derived from the protein isoform sequences; (k) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; (1) generating a first set of antigenic cell surface antigen sequences based on the first RNA-seq data set and a second set of antigenic cell surface antigen sequences based on the second RNA-seq data set ranked by B cell antibody accessibility and T cell antigenicity; and (m) determining unique antigenic cell surface antigen sequences by comparing the first set of antigenic cell surface antigen sequences and the second set of antigenic cell surface antigen sequences and selecting cell surface antigen sequences present in one set and not the other set; selecting one or more unique cell surface antigen sequences.

Exemplary cell surface antigens and protein isoforms identified using these methods in EXAMPLE 3 are listed in TABLE 1 and TABLE 2.

TABLE 1 exemplary cell surface antigens resulting from alternative splice events in the human genome.

TABLE 1

| PEP ID NO: | SEQ ID NO: | Amino Acid Sequence |
|---|---|---|
| PEP1 | 1 | ACIREPR |
| PEP2 | 2 | ARPCPAR |
| PEP3 | 3 | AVAAPTK |
| PEP4 | 4 | AWCSEGR |
| PEP5 | 5 | DENSQLGR |
| PEP6 | 6 | DSWEGGR |
| PEP7-1 | 7 | ENLTSIVLNSKYIPK |
| PEP8-1 | 8 | EWGQGPR |
| PEP9 | 9 | FFESLRK |
| PEP10 | 10 | FLSILCS |
| PEP11-1 | 11 | GGFTFGK |
| PEP12 | 12 | HHPQPAL |
| PEP13 | 13 | LEEESFR |
| PEP14 | 14 | LGKQTAAK |
| PEP15-1 | 15 | LLCLQGR |
| PEP16 | 16 | LRMEELWR |
| PEP17-1 | 17 | LYWMFVR |
| PEP18 | 18 | NTGAVCR |
| PEP19-1 | 19 | QQANMLPPTERVL |
| PEP20-1 | 20 | RASLCGK |
| PEP21-1 | 21 | RLSQLPLK |
| PEP22-1 | 22 | SAQTGLS |
| PEP23 | 23 | SGSEEVR |
| PEP24 | 24 | SPDSTLR |
| PEP25 | 25 | SPGYGSK |
| PEP26 | 26 | SSGLGLRR |
| PEP27 | 27 | VWGAGRR |

TABLE 2 protein isoforms resulting from alternative splice events in the human genome identified in EXAMPLE 3.

TABLE 2

| PEP ID | | | SEQ ID |
|---|---|---|---|
| NO: | Peptide | Full Isoform Sequence | NO: |
| PEP1 | ACIREP | MCAEDTLQGILTPACIREPRSCGRGSVERERSSGDGPQGLR | 28 |
| | R | AGGRGSVESGERSSGDDPQRRLAKCGCASPPCPRRKLSHSK | |
| | | GRPEGSAGRLCTDTCPPRGSPPAPGPCRLVLRV | |
| PEP2 | ARPCPA | MAAGGLSRSERKAAERVRRLREEQQRERLRQVRRRRSPARP | 29 |
| | R | CPARAAAHRPPARRCRAS | |
| PEP3 | AVAAPT | MERVVVSMQDPDQGVKMRSQRLLVTVIPHAVTGSDVVQWLA | 30 |
| | K | QKFCVSEEEALHLGAVLVQHGYIYPLRDPRSLMLRPDETPY | |
| | | RFQTPYFWTSTLRPAAELDYAIYLAKKNIRKRGTLVDYEKD | |
| | | CYDRLHKKINHAWDLVLMQAREQLRAAKQRSKGDRLVIACQ | |
| | | EQTYWLVNRPPPGAPDVLEQGPGRGSCAASRVLMTKSADFH | |
| | | KREIEYFRKALGRTRVKSSVCLEAVAAPTKLRVERWGFSFR | |
| | | ELLEDPVGRAHFMDFLGKEFSGENLSFWEACEELRYGAQAQ | |
| | | VPTLVDAVYEQFLAPGAAHWVNIDSRTMEQTLEGLRQPHRY | |
| | | VLDDAQLHIYMLMKKDSYPRFLKSDMYKALLAEAGIPLEMK | |
| | | RRVFPFTWRPRHSSPSPALLPTPVEPTAACGPGGGDGVA | |
| PEP4 | AWCSEG | MPPPRTGRGLLWLGLVLSSVCVALGSETQANSTTDALNVLL | 31 |
| | R | IIVDDLRPSLGCYGDKLVRSPNIDQLASHSLLFQNAFAQVC | |
| | | LGTSSCGCVLLRALRVGGGELQLLSDGTDCAGHLVRGKHSL | |
| | | ENGSGENLVFHSWESLTFKGGLLLGCKVRPGPDVFMAALAS | |
| | | FLPERALAWCSEGRGAAEGHPQVCRLGLR | |
| PEP5 | DENSQL | MDRTETRFRKRGQITGKITTSRQPHPQNEQSPQRSTSGYPL | 32 |
| | GR | QEVVDDEMLGPSGTQRARDQGRTGSSVRRTEREKNGEGKER | |
| | | HMGLSRGENQKDGLEKPAVCKSGEDGEWFGVLGRGLRSLGW | |
| | | KRKREWSDESEEEPEKELAPEPEETWVVEMLCGLKMKLKQQ | |
| | | RVSSILPEHHKDFNSQLGRRIPQRAPPILFFLKRGNFQ | |
| PEP6 | DSWEGG | MVLAQGLLSMALLALCWERSLAGAEETIPLQTLRCYNDYTS | 33 |
| | R | HITCRWADTQDAQRLVNVTLIRRVNEDLLEPVSCDLSDDMP | |
| | | WSACPHPRCVPRRCVIPCQSFVVTDVDYFSFQPDRPLGTRL | |
| | | TVTLTQHVQPPEPRDLQISTDQDHFLLTWSVALGSPQSHWL | |
| | | SPGDLEFEVVYKRLQDSWEGGRVLPSAEGGARQPPHQAPLP | |
| | | DSRARPRDPRPIHRLCSAKEGRETHKELSEHPDGPSIPQRD | |
| | | QGWRQLQPALGNNENAIRTHRPHI | |
| PEP7-1 | ENLTSI | MENNMVELSKLQEYKLELDERAMQAVEKLEEIHLQKQAQYE | 34 |
| | VLNSKY | KOLEQLNKDNTASLNMKELTLKDVECKFSKMKTTYEEVTTK | |
| | IPK | LEEYKEAFAAALNANNSMSKKLTKSNKKIAMISTKLLMEKE | |
| | | WVKYFLSTLPTRRGQESPCVENLTSIVLNSKYIPKMTVRIP | |
| | | TSNPQTSNNCQNYLTEMELDCVEQIIRETKRSMLPKFIN | |
| PEP7-2 | ENLTSI | MRVGGVRPPRATDMKKDVQILVVGEPRVGKTSLIMSLVSEE | 35 |
| | VLNSKY | FPEEVPPRAEEITIPADVTPERVPTHIVDYSEAEQSNEQLH | |
| | IPK | QEISQANVVCIVYAVNNKHSIDKKQAQYEKQLEQLNKDNTA | |
| | | SLNMKELTLKDVECKFSKMKTTYEEVTTKLEEYKEAFAAAL | |
| | | NANNSMSKKLTKSNKKIAMISTKLLMEKEWVKYFLSTLPTR | |
| | | RGQESPCVENLTSIVLNSKYIPKMTVRIPTSNPQTSNNCQN | |
| | | YLTEVSY | |
| PEP8-1 | EWGQGP | MKVLRKAKIVRNAKDTAHTRAERNILESVKHPFIVELAYAF | 36 |
| | R | QTGGKLYLILECLSGGELFTHLEREGIFLEDTACFYLAEIT | |
| | | LALGHLHSQGITYRDLKPENIMLSSQGHIKLTDFGLCKESI | |
| | | HEGAVTHTFCGTIEYMAPEILVRSGHNRAVDWWSLGALMYD | |
| | | MLTGSPPFTAENRKKTMDKIIRGKLALPPYLTPDARDLVKK | |
| | | FLKRNPSQRIGGGPGDAADVQVGLGPPPGVGLSLQGCREWG | |
| | | QGPRAEGVTGGQAG | |
| PEP8-2 | EWGQGP | MAAVFDLDLETEEGSEGEGEPELSPADACPLAELRAAGLEP | 37 |
| | R | VGHYEEVELTETSVNVGPERIGPHCFELLRVLGKGGYGKVF | |
| | | QVRKVQGTNLGKIYAMKVLRKAKIVRNAKDTAHTRAERNIL | |
| | | ESVKHPFIVELAYAFQTGGKLYLILECLSGGELFTHLEREG | |
| | | IFLEDTACFYLAEITLALGHLHSQGITYRDLKPENIMLSSQ | |
| | | GHIKLTDFGLCKESIHEGAVTHTFCGTIEYMAPEILVRSGH | |
| | | NRAVDWWSLGALMYDMLTGSPPFTAENRKKTMDKIIRGKLA | |
| | | LPPYLTPDARDLVKKFLKRNPSQRIGGGPGDAADVQVGLGP | |
| | | PPGVGLSLQGCREWGQGPRAEGVTGGQAG | |
| PEP9 | FFESLR | MVRSGNKAAVVLCMDVGFTMSNSIPGIESPFEQAKKVITME | 38 |
| | K | VQRQVFAENKDEIALVLFGTDGTDNPLSGGDQYQNITVHRH | |
| | | LMLPDFDLLEDIESKIQPGSQQADFLDALIVSMDVIQHETI | |
| | | GKKFEKRHIEIFTDLSSRFSKSQLDIIIHSLKKCDISLQFF | |
| | | ESLRKLCVFKKIERHSIHWPCRLTIGSNLSIRIAAYKSILQ | |
| | | ERVKKTWTVVDAKTLKKEDIQKETVYCLNDDDETEVLKEDI | |
| | | IQGFRYGSDIVPFSKVDEEQMKYKSEGKCFSVLGFCKSSQV | |
| | | QRRFFMGNQVLKVFAARDDEAAAVALSSLIHALDDLDMVAI | |
| | | VRYAYDKRANPQVGVAFPHIKHNYECLVYVQLPFMEDLRQY | |
| | | MFSSLKNSKKYAPTEAQLNAVDALIDSMSLAKKDEKTDTLE | |
| | | DLFPTTKIPNPRFQRLFQCLLHRALHPREPLPPIQQHIWNM | |
| | | LNPPAEVTTKSQIPLSKIKTLFPLIEAKKKDQVTAQEIFQD | |
| | | NHEDGPTAKKLKTEQGGAHFSVSSLAEGSVTSVGSVNPAEN | |
| | | FRVLVKQKKASFEEASNQLINHIEQFLDTNETPYFMKSIDC | |
| | | IRAFREEAIKFSEEQRFNNFLKALQEKVEIKQLNHFWEIVV | |
| | | QDGITLITKEEASGSSVTAEEAKKFLAPKDKPSGDTAAVFE | |
| | | EGGDVDDLLDMI | |
| PEP10 | FLSILC | MNHSPLKTALAYECFQDQDNSTLALPSDQKMKTGTSGRQRV | 40 |
| | S | QEQVMMTVKRQKSKSSQSSTLSHSNRGSMYDGLADNYNYGT | |
| | | TSRSSYYSKFQAGNGSWGYPTYNGTLKREPDNRRFSSYSQM | |
| | | ENWSRHYPRGSCNTTGAGSDICEMQKIKASRSEPDLYCDPR | |
| | | GTLRKGTLGSKGQKTTQNRYSFYSTCSGQKAIKKCPVRPPS | |
| | | CASKQDPVYIPPISCNKDLSFGHSRASSKICSEDIECSGLT | |
| | | IPKAVQYLSSQDEKYQAIGAYYIQHTCFQDESAKQQVYQLG | |
| | | GICKLVDLLRSPNQNVQQAAAGALRNLVFRSTTNKLETRRQ | |
| | | NGIREAVSLLRRTGNAEIQKQLTGLLWNLSSTDELKEELIA | |
| | | DALPVLADRVIIPFSGWCDGNSNMSREVVDPEVFFNATGCL | |
| | | RNLSSADAGRQTMRNYSGLIDSLMAYVQNCVAASRCDDKSV | |
| | | ENCMCVLHNLSYRLDAEVPTRYRQLEYNARNAYTEKSSTGC | |
| | | FSNKSDKMMNNNYDCPLPEEETNPKGSGWLYHSDAIRTYLN | |
| | | LMGKSKKDATLEACAGALQNLTASKGLMSSGMSQLIGLKEK | |
| | | GLPQIARLLQSGNSDVVRSGASLLSNMSRHPLLHRVMGRYD | |
| | | PAEKPSGLAGWGFLSILCSIWESSQETEKKPKNCG | |
| PEP11- | GGFTFG | MDLEGDRNGGAKKKNFFKLNNKSEKDKKEKKPTVSVFSMFR | 41 |
| 1 | K | YSNWLDKLYMVVGTLAAIIHGAGLPLMMLVFGEMTDIFANA | |
| | | GNLEDLMSNITNRSDINDTGFFMNLEEDMTRYAYYYSGIGA | |
| | | GVLVAAYIQVSFWCLAAGRQIHKIRKQFFHAIMRQEIGWFD | |
| | | VHDVGELNTRLTDDVSKINEGIGDKIGMFFQSMATFFTGFI | |
| | | VGFTRGWKLTLVILAISPVLGLSAAVWAKILSSFTDKELLA | |
| | | YAKAGAVAEEVLAAIRTVIAFGGQKKELERYNKNLEEAKRI | |
| | | GIKKAITANISIGAAFLLIYASYALAFWYGTTLVLSGEYSI | |
| | | GQVLTVFFSVLIGAFSVGQASPSIEAFANARGAAYEIFKII | |
| | | DNKPSIDSYSKSGHKPDNIKGNLEFRNVHFSYPSRKEVKIL | |
| | | KGLNLKVQSGQTVALVGNSGCGKSTTVQLMQRLYDPTEGMV | |
| | | SVDGQDIRTINVRFLREIIGVVSQEPVLFATTIAENIRYGR | |
| | | ENVTMDEIEKAVKEANAYDFIMKLPHKFDTLVGERGAQLSG | |
| | | GQKORIAIARALVRNPKILLLDEATSALDTESEAVVQVALD | |
| | | KARKGRTTIVIAHRLSTVRNADVIAGFDDGVIVEKGNHDEL | |
| | | MKEKGIYFKLVTMQTAGNEVELENAADESKSEIDALEMSSN | |
| | | DSRSSLIRKRSTRRSVRGSQAQDRKLSTKEALDESIPPVSF | |
| | | WRIMKLNLTEWPYFVVGVFCAIINGGLQPAFAIIFSKIIGG | |
| | | FTFGKAGEILTKRLRYMVFRSMLRQDVSWFDDPKNTTGALT | |
| | | TRLANDAAQVKGAIGSRLAVITQNIANLGTGIIISFIYGWQ | |
| | | LTLLLLAIVPIIAIAGVVEMKMLSGQALKDKKELEGSGKIA | |
| | | TEAIENFRTVVSLTQEQKFEHMYAQSLQVPYRNSLRKAHIF | |
| | | GITFSFTQAMMYFSYAGCFRFGAYLVAHKLMSFEDVLLVFS | |
| | | AVVFGAMAVGQVSSFAPDYAKAKISAAHIIMIIEKTPLIDS | |
| | | YSTEGLMPNTLEGNVTFGEVVFNYPTRPDIPVLQGLSLEVK | |
| | | KGQTLALVGSSGCGKSTVVQLLERFYDPLAGKVLLDGKEIK | |
| | | RLNVQWLRAHLGIVSQEPILFDCSIAENIAYGDNSRVVSQE | |
| | | EIVRAAKEANIHAFIESLPNKYSTKVGDKGTQLSGGQKQRI | |
| | | AIARALVROPHILLLDEATSALDTESEKVVQEALDKAREGR | |
| | | TCIVIAHRLSTIQNADLIVVFQNGRVKEHGTHQQLLAQKGI | |
| | | YFSMVSVQAGTKRQ | |
| PEP11- | GGFTFG | MDLEGDRNGGAKKKNFFKLNNKSEKDKKEKKPTVSVFSMFR | 42 |
| 2 | K | YSNWLDKLYMVVGTLAAIIHGAGLPLMMLVFGEMTDIFANA | |
| | | GNLEDLMSNITNRSDINDTGFFMNLEEDMTRYAYYYSGIGA | |
| | | GVLVAAYIQVSFWCLAAGRQIHKIRKQFFHAIMRQEIGWFD | |
| | | VHDVGELNTRLTDDVSKINEGIGDKIGMFFQSMATFFTGFI | |
| | | VGFTRGWKLTLVILAISPVLGLSAAVWAKILSSFTDKELLA | |
| | | YAKAGAVAEEVLAAIRTVIAFGGQKKELERYNKNLEEAKRI | |
| | | GIKKAITANISIGAAFLLIYASYALAFWYGTTLVLSGEYSI | |
| | | GQVLTVFFSVLIGAFSVGQASPSIEAFANARGAAYEIFKII | |
| | | DNKPSIDSYSKSGHKPDNIKGNLEFRNVHFSYPSRKEVKIL | |
| | | KGLNLKVQSGQTVALVGNSGCGKSTTVQLMQRLYDPTEGMV | |
| | | SVDGQDIRTINVRELREIIGVVSQEPVLFATTIAENIRYGR | |
| | | ENVTMDEIEKAVKEANAYDFIMKLPHKFDTLVGERGAQLSG | |
| | | GQKQRIAIARALVRNPKILLLDEATSALDTESEAVVQVALD | |
| | | KARKGRTTIVIAHRLSTVRNADVIAGFDDGVIVEKGNHDEL | |
| | | MKEKGIYFKLVTMQTAGNEVELENAADESKSEIDALEMSSN | |
| | | DSRSSLIRKRSTRRSVRGSQAQDRKLSTKEALDESIPPVSF | |
| | | WRIMKLNLTEWPYFVVGVFCAIINGGLQPAFAIIFSKIIGG | |
| | | FTFGKAGEILTKRLRYMVFRSMLRQDVSWFDDPKNTTGALT | |
| | | TRLANDAAQVKGAIGSRLAVITQNIANLGTGIIISFIYGWQ | |
| | | LTLLLLAIVPIIAIAGVVEMKMLSGQALKDKKELEGSGKIA | |
| | | TEAIENFRTVVSLTQEQKFEHMYAQSLQVPYRNSLRKAHIF | |
| | | GITFSFTQAMMYFSYAGCFRFGAYLVAHKLMSFEDVLLVFS | |
| | | AVVFGAMAVGQVSSFAPDYAKAKISAAHIIMIIEKTPLIDS | |
| | | YSTEGLMPNTLEGNVTFGEVVFNYPTRPDIPVLQGLSLEVK | |
| | | KGQTLALVGSSGCGKSTVVQLLERFYDPLAGKVLLDGKEIK | |
| | | RLNVQWLRAHLGIVSQEPILFDCSIAENIAYGDNSRVVSQE | |
| | | EIVRAAKEANIHAFIESLPNKYSTKVGDKGTQLSGGQKQRI | |
| | | AIARALVRQPHILLLDEATSALDTESEKVVQEALDKAREGR | |
| | | TCIVIAHRLSTIQNADLIVVFQNGRVKEHGTHQQLLAQKGI | |
| | | YFSMVSVQAGTKRQ | |
| PEP11- | GGFTFG | MDLEGDRNGGAKKKNFFKLNNKSEKDKKEKKPTVSVFSMFR | 41 |
| 3 | K | YSNWLDKLYMVVGTLAAIIHGAGLPLMMLVFGEMTDIFANA | |
| | | GNLEDLMSNITNRSDINDTGFFMNLEEDMTRYAYYYSGIGA | |
| | | GVLVAAYIQVSFWCLAAGRQIHKIRKQFFHAIMRQEIGWFD | |
| | | VHDVGELNTRLTDDVSKINEGIGDKIGMFFQSMATFFTGFI | |
| | | VGFTRGWKLTLVILAISPVLGLSAAVWAKILSSFTDKELLA | |
| | | YAKAGAVAEEVLAAIRTVIAFGGQKKELERYNKNLEEAKRI | |
| | | GIKKAITANISIGAAFLLIYASYALAFWYGTTLVLSGEYSI | |
| | | GQVLTVFFSVLIGAFSVGQASPSIEAFANARGAAYEIFKII | |
| | | DNKPSIDSYSKSGHKPDNIKGNLEFRNVHFSYPSRKEVKIL | |
| | | KGLNLKVQSGQTVALVGNSGCGKSTTVQLMQRLYDPTEGMV | |
| | | SVDGQDIRTINVRELREIIGVVSQEPVLFATTIAENIRYGR | |
| | | ENVTMDEIEKAVKEANAYDFIMKLPHKFDTLVGERGAQLSG | |
| | | GQKQRIAIARALVRNPKILLLDEATSALDTESEAVVQVALD | |
| | | KARKGRTTIVIAHRLSTVRNADVIAGFDDGVIVEKGNHDEL | |
| | | MKEKGIYFKLVTMQTAGNEVELENAADESKSEIDALEMSSN | |
| | | DSRSSLIRKRSTRRSVRGSQAQDRKLSTKEALDESIPPVSF | |
| | | WRIMKLNLTEWPYFVVGVFCAIINGGLQPAFAIIFSKIIGG | |
| | | FTFGKAGEILTKRLRYMVFRSMLRQDVSWFDDPKNTTGALT | |
| | | TRLANDAAQVKGAIGSRLAVITQNIANLGTGIIISFIYGWQ | |
| | | LTLLLLAIVPIIAIAGVVEMKMLSGQALKDKKELEGSGKIA | |
| | | TEAIENFRTVVSLTQEQKFEHMYAQSLQVPYRNSLRKAHIF | |
| | | GITFSFTQAMMYFSYAGCFRFGAYLVAHKLMSFEDVLLVFS | |
| | | AVVFGAMAVGQVSSFAPDYAKAKISAAHIIMIIEKTPLIDS | |
| | | YSTEGLMPNTLEGNVTFGEVVFNYPTRPDIPVLQGLSLEVK | |
| | | KGQTLALVGSSGCGKSTVVQLLERFYDPLAGKVLLDGKEIK | |
| | | RLNVQWLRAHLGIVSQEPILFDCSIAENIAYGDNSRVVSQE | |
| | | EIVRAAKEANIHAFIESLPNKYSTKVGDKGTQLSGGQKQRI | |
| | | AIARALVRQPHILLLDEATSALDTESEKVVQEALDKAREGR | |
| | | TCIVIAHRLSTIQNADLIVVFQNGRVKEHGTHQQLLAQKGI | |
| | | YFSMVSVQAGTKRQ | |
| PEP12 | HHPQPA | MEATGVLPFVRGVDLSGNDFKGGYFPENVKAMTSLRWLKLN | 43 |
| | L | RTGLCYLPEELAALQKLEHLSVSHNNLTTLHGELSSLPSLR | |
| | | AIVARANSLKNSGVPDDIFKLDDLSVLHRHHPQPALHQPH | |
| PEP13 | LEEESF | MSAFCLGLVGRASAPAEPDSACCMELPAAAGDAVRSPAAAA | 44 |
| | R | ALIFPGGSGELELALEEELALLAAGERPSDPGEHPQAEPGS | |
| | | LAEGAGPQPPPSQDPELLSVIRQKEKDLVLAARLGKALLER | |
| | | NQDMSRQYEQMHKELTDKLEHLEQEKHELRRRFENREGEWE | |
| | | GRVSELESDVKQLQDELERQQIHLREADREKSRAVQELSEQ | |
| | | NQRLLDQLSRVGMVTAMDALEEESFRLSSSTSDAEFDAVVV | |
| | | YLEDIIMDDRFPIITEKLHGQVLLGLASEVERQLSMQVHAL | |
| | | REDFREKNSSTNQHIIRLESLQAEIKMLSDRKRELEHRLSA | |
| | | TLEENDLLQGTVEELQDRVLILERQGHDKDLQLHQSQLELQ | |
| | | EVRLSCRQLQVKVEELTEERSLQSSAATSTSLLSEIEQSME | |
| | | AEELEQEREQLRLOLWEAYCQVRYLCSHLRGNDSADSAVST | |
| | | DSSMDESSETSSAKDVPAGSLRTALNELKRLIQSIVDGMEP | |
| | | TVTLLSVEMTALKEERDRLRVTSEDKEPKEQLQKAIRDRDE | |
| | | AIAKKNAVELELAKCRMDMMSLNSQLLDAIQQKLNLSQQLE | |
| | | AWQDDMHRVIDRQLMDTHLKERSQPAAALCRGHSAGRGDEP | |
| | | SIAEGKRLESFFRKI | |
| PEP14 | LGKQTA | MESIFHEKQEGSLCAQHCLNNLLQGEYFSPVELSSIAHQLD | 45 |
| | AK | EEERMRMAEGGVTSEDYRTFLQQPSGNMDDSGFFSIQVISN | |
| | | ALKVWGLELILFNSPEYQRLRIDPINERSFICNYKEHWFTV | |
| | | RKLGKQTAAKAATAAAAAAAGGPIRTEFTSM | |
| PEP15- | LLCLQG | MLEYALKQERAKYHKLKFGTDLNQGEKKADVSEQVSNGPVE | 46 |
| 1 | R | SVTLENSPLVWKEGROLLRQYLEEVGYTDTILDMRSKRVRS | |
| | | LLGRSLELNGAVEPSEGAPRAPPGPAGLSGGESLLVKQIEE | |
| | | QIKRNAAGKDGKERLGGSVLGQIPFLQNCEDEDSDEDDELD | |
| | | SVQHKKORVKLPSKALVPEMEDEDEEDDSEDAINEFDFLGS | |
| | | GEDGEGAPDPRRCTVDGSPHELESRRVKLQGILADLRDVDG | |
| | | LPPKVTGPPPGTPQPRPHEGKRHPPPGPSPAGPWQREAAEL | |
| | | SPGLLCLQGRGPASSLQPPCPKSSSVCLSPSLFVCPALSVS | |
| | | LSLSSCDSSSCPLPVSLSCSLSLSLPLSVILSLPGPLCPSL | |
| | | PSPYQVPLASPQTSSSWTLSGAGR | |
| PEP15- | LLCLQG | MLEYALKQERAKYHKLKFGTDLNQGEKKADVSEQVSNGPVE | 46 |
| 2 | R | SVTLENSPLVWKEGRQLLRQYLEEVGYTDTILDMRSKRVRS | |
| | | LLGRSLELNGAVEPSEGAPRAPPGPAGLSGGESLLVKQIEE | |
| | | QIKRNAAGKDGKERLGGSVLGQIPFLQNCEDEDSDEDDELD | |
| | | SVQHKKQRVKLPSKALVPEMEDEDEEDDSEDAINEFDFLGS | |
| | | GEDGEGAPDPRRCTVDGSPHELESRRVKLQGILADLRDVDG | |
| | | LPPKVTGPPPGTPQPRPHEGKRHPPPGPSPAGPWQREAAEL | |
| | | SPGLLCLQGRGPASSLQPPCPKSSSVCLSPSLFVCPALSVS | |
| | | LSLSSCDSSSCPLPVSLSCSLSLSLPLSVILSLPGPLCPSL | |
| | | PSPYQVPLASPQTSSSWTLSGAGR | |
| PEP15- | LLCLQG | MLEYALKQERAKYHKLKFGTDLNQGEKKADVSEQVSNGPVE | 46 |
| 3 | R | SVTLENSPLVWKEGRQLLRQYLEEVGYTDTILDMRSKRVRS | |
| | | LLGRSLELNGAVEPSEGAPRAPPGPAGLSGGESLLVKQIEE | |
| | | QIKRNAAGKDGKERLGGSVLGQIPFLQNCEDEDSDEDDELD | |
| | | SVQHKKQRVKLPSKALVPEMEDEDEEDDSEDAINEFDFLGS | |
| | | GEDGEGAPDPRRCTVDGSPHELESRRVKLQGILADLRDVDG | |
| | | LPPKVTGPPPGTPQPRPHEGKRHPPPGPSPAGPWQREAAEL | |
| | | SPGLLCLQGRGPASSLQPPCPKSSSVCLSPSLFVCPALSVS | |
| | | LSLSSCDSSSCPLPVSLSCSLSLSLPLSVILSLPGPLCPSL | |
| | | PSPYQVPLASPQTSSSWTLSGAGR | |
| PEP15- | LLCLQG | MLEYALKQERAKYHKLKFGTDLNQGEKKADVSEQVSNGPVE | 46 |
| 4 | R | SVTLENSPLVWKEGRQLLRQYLEEVGYTDTILDMRSKRVRS | |
| | | LLGRSLELNGAVEPSEGAPRAPPGPAGLSGGESLLVKQIEE | |
| | | QIKRNAAGKDGKERLGGSVLGQIPFLQNCEDEDSDEDDELD | |
| | | SVQHKKQRVKLPSKALVPEMEDEDEEDDSEDAINEFDFLGS | |
| | | GEDGEGAPDPRRCTVDGSPHELESRRVKLQGILADLRDVDG | |
| | | LPPKVTGPPPGTPQPRPHEGKRHPPPGPSPAGPWQREAAEL | |
| | | SPGLLCLQGRGPASSLQPPCPKSSSVCLSPSLFVCPALSVS | |
| | | LSLSSCDSSSCPLPVSLSCSLSLSLPLSVILSLPGPLCPSL | |
| | | PSPYQVPLASPQTSSSWTLSGAGR | |
| PEP16 | LRMEEL | MRWRTILLQYCFLLITCLLTALEAVPIDIDKTKVQNIHPVE | 47 |
| | WR | SAKIEPPDTGLYYDEYLKQVIDVLETDKHFREKLQKADIEE | |
| | | IKSGRLSKELDLVSHHVRTKLDELKRQEVGRLRMLIKAKLD | |
| | | SLQDIGMDHQALLKQFDHLNHLNPDKFESTDLDMLIKAATS | |
| | | DLEHYDKTRHEEFKKYEMMKEHERREYLKTLNEEKRKEEES | |
| | | KFEEMKKKHENHPKVNHPGSKDQLKEVWEETDGLDPNDFDP | |
| | | KTFFKLHDVNSDGFLDEQELEALFTKELEKVYDPKNEEDDM | |
| | | VEMEEERLRMREHVMNEVDINKDRLVTLEEFLKATEKKEFL | |
| | | EPDSWETLDQQQFFTEEELKEYENIIALQENELKKKADELQ | |
| | | KOKEELQRQHDQLEAQKLEYHQFQDLRMEELWRLKVEDGSP | |
| | | FQGQ | |
| PEP17- | LYWMFV | MFFLWFLRLYLHYLGQWLFLQAISTPVTKFHFSLHIVELCY | 48 |
| 1 | R | PTSSLHIGEELPVVVMGPLMLNAILLLLVLIRWGCQLLFAS | |
| | | CPDVLSKLIITMGLWTILDPLAVFILDTLLGRLTDNEETPV | |
| | | ADAAKLYWMFVRTVQPGILGVVITVLLYILLFVISSLILYL | |
| | | YCLRLHNDSWILDAFQRIHSEETKFFIPYDLEISNQELSYI | |
| | | VK | |
| PEP17- | LYWMFV | MFFLWFLRLYLHYLGQWLFLQAISTPVTKFHFSLHIVELCY | 48 |
| 2 | R | PTSSLHIGEELPVVVMGPLMLNAILLLLVLIRWGCQLLFAS | |
| | | CPDVLSKLIITMGLWTILDPLAVFILDTLLGRLTDNEETPV | |
| | | ADAAKLYWMFVRTVQPGILGVVITVLLYILLFVISSLILYL | |
| | | YCLRLHNDSWILDAFQRIHSEETKFFIPYDLEISNQELSYI | |
| | | VK | |
| PEP18 | NTGAVC | MPSSMGGGGGGSPSPVELRGALVGSVDPTLREQQLQQELLA | 49 |
| | R | LKQQQQLQKOLLFAEFQKQHDHLTRQHEVQLQKHLKQQQEM | |
| | | LAAKQQQEMLAAKROQELEQQROREQQRQEELEKQRLEQQL | |
| | | LILRNKEKSKESAIASTEVKLRLQEFLLSKSKEPTPGGLNH | |
| | | SLPQHPKCWGAHHASLDQSSPPQSGPPGTPPSYKLPLPGPY | |
| | | DSRDDFPLRKTASEPNLKVRSRLKQKVAERRSSPLLRRKDG | |
| | | TVISTFKKRAVEITGAGPGASSVCNSAPGSGPSSPNSSHST | |
| | | IAENGFTGSVPNIPTEMLPQHRALPLDSSPNQFSLYTSPSL | |
| | | PNISLGLQATVTVTNSHLTASPKLSTQQEAERQALQSLRQG | |
| | | GTLTGKFMSTSSIPGCLLGVALEGDGSPHGHASLLQHVLLL | |
| | | EQARQQSTLIAVPLHGQSPLVTGERVATSMRTVGKLPRHRP | |
| | | LSRTOSSPLPQSPQALQQLVMQQQHQQFLEKQKQQQLQLGK | |
| | | ILTKTGELPRQPTTHPEETEEELTEQQEVLLGEGALTMPRE | |
| | | GSTESESTQEDLEEEDEEDDGEEEEDCIQVKDEEGESGAEE | |
| | | GPDLEEPGAGYKKLFSDAQPLQPLQVYQAPLSLATVPHQAL | |
| | | GRTQSSPAAPGGMKSPPDQPVKHLFTTGVVYDTFMLKHQCM | |
| | | CGNTHVHPEHAGRIQSIWSRLQETGLLSKCERIRGRKATLD | |
| | | EIQTVHSEYHTLLYGTSPLNRQKLDSKKLLGPISQKMYAVL | |
| | | PCGGIGVDSDTVWNEMHSSSAVRMAVGCLLELAFKVAAGEL | |
| | | KNGFAIIRPPGHHAEESTAMGFCFFNSVAITAKLLQQKLNV | |
| | | GKVLIVDWDIHHGNGTQQAFYNDPSVLYISLHRYDNGNFFP | |
| | | GSGAPEEVGGGPGVGYNVNVAWTGGVDPPIGDVEYLTAFRT | |
| | | VVMPIAHEFSPDVVLVSAGFDAVEGHLSPLGGYSVTARCFG | |
| | | HLTRQLMTLAGGRVVLALEGGHDLTAICDASEACVSALLSV | |
| | | EANTGAVCRSSPLVWAGPCERPKQVRPRRPRL | |
| PEP19- | QQANML | MSSVSPIQIPSRLPLLLTHEGVLLPGSTMRTSVDSARNLQL | 50 |
| 1 | PPTERV | VRSRLLKGTSLQSTILGVIPNTPDPASDAQDLPPLHRIGTA | |
| | L | ALAVQVVGSNWPKPHYTLLITGLCRFQIVQVLKEKPYPIAE | |
| | | VEQLDRLEEFPNTCKMREELGELSEQFYKYAVQLVEMLDMS | |
| | | VPAVAKLRRLLDSLPREALPDILTSIIRTSNKEKLQILDAV | |
| | | SLEERFKMTIPLLVRQIEGLKLLQKTRKPKQDDDKRVIAIR | |
| | | PIRRITHISGTLEDEDEDEDNDDIVMLEKKIRTSSMPEQAH | |
| | | KVCVKEIKRLKKMPQSMPEYALTRNYLELMVELPWNKSTTD | |
| | | RLDIRAARILLDNDHYAMEKLKKRVLEYLAVRQLKNNLKGP | |
| | | ILCFVGPPGVGKTSVGRSVAKTLGREFHRIALGGVCDQSDI | |
| | | RGHRRTYVGSMPGRIINGLKTVGVNNPVFLLDEVDKLGKSL | |
| | | QGDPAAALLEVLDPEQNHNFTDHYLNVAFDLSQVLFIATAN | |
| | | TTATIPAALLDRMEIIQVPGYTQEEKIEIAHRHLIPKQLEQ | |
| | | HGLTPQQIQIPQVTTLDIITRYTREAGVRSLDRKLGAICRA | |
| | | VAVKVAEGQHKEAKLDRSDVTEREGCREHILEDEKPESISD | |
| | | TTDLALPPEMPILIDFHALKDILGPPMYEMEVSQRLSQPGV | |
| | | AIGLAWTPLGGEIMFVEASRMDGEGQLTLTGQLGDVMKESA | |
| | | HLAISWLRSNAKKYQLTNAFGSFDLLDNTDIHLHFPAGAVT | |
| | | KDGPSAGVTIVTCLASLFSGRLVRSDVAMTGEITLRGLVLP | |
| | | VGGIKDKVLAAHRAGLKQVIIPRRNEKDLEGIPGNVRQDLS | |
| | | FVTASCLDEVLNAAFDGGFTVKTRPGLLNSKLGRKYQKGLN | |
| | | RQQANMLPPTERVLGWQTDGCLIFCETEVLNTGQKMFDCHF | |
| | | NTWK | |
| PEP19- | QQANML | MSSVSPIQIPSRLPLLLTHEGVLLPGSTMRTSVDSARNLQL | 50 |
| 2 | PPTERV | VRSRLLKGTSLQSTILGVIPNTPDPASDAQDLPPLHRIGTA | |
| | L | ALAVQVVGSNWPKPHYTLLITGLCRFQIVQVLKEKPYPIAE | |
| | | VEQLDRLEEFPNTCKMREELGELSEQFYKYAVQLVEMLDMS | |
| | | VPAVAKLRRLLDSLPREALPDILTSIIRTSNKEKLQILDAV | |
| | | SLEERFKMTIPLLVRQIEGLKLLQKTRKPKQDDDKRVIAIR | |
| | | PIRRITHISGTLEDEDEDEDNDDIVMLEKKIRTSSMPEQAH | |
| | | KVCVKEIKRLKKMPQSMPEYALTRNYLELMVELPWNKSTTD | |
| | | RLDIRAARILLDNDHYAMEKLKKRVLEYLAVRQLKNNLKGP | |
| | | ILCFVGPPGVGKTSVGRSVAKTLGREFHRIALGGVCDQSDI | |
| | | RGHRRTYVGSMPGRIINGLKTVGVNNPVFLLDEVDKLGKSL | |
| | | QGDPAAALLEVLDPEQNHNFTDHYLNVAFDLSQVLFIATAN | |
| | | TTATIPAALLDRMEIIQVPGYTQEEKIEIAHRHLIPKQLEQ | |
| | | HGLTPQQIQIPQVTTLDIITRYTREAGVRSLDRKLGAICRA | |
| | | VAVKVAEGQHKEAKLDRSDVTEREGCREHILEDEKPESISD | |
| | | TTDLALPPEMPILIDFHALKDILGPPMYEMEVSQRLSQPGV | |
| | | AIGLAWTPLGGEIMFVEASRMDGEGQLTLTGQLGDVMKESA | |
| | | HLAISWLRSNAKKYQLTNAFGSFDLLDNTDIHLHFPAGAVT | |
| | | KDGPSAGVTIVTCLASLFSGRLVRSDVAMTGEITLRGLVLP | |
| | | VGGIKDKVLAAHRAGLKQVIIPRRNEKDLEGIPGNVRQDLS | |
| | | FVTASCLDEVLNAAFDGGFTVKTRPGLLNSKLGRKYQKGLN | |
| | | RQQANMLPPTERVLGWQTDGCLIFCETEVLNTGQKMFDCHF | |
| | | NTWK | |
| PEP19- | QQANML | MSSVSPIQIPSRLPLLLTHEGVLLPGSTMRTSVDSARNLQL | 50 |
| 3 | PPTERV | VRSRLLKGTSLQSTILGVIPNTPDPASDAQDLPPLHRIGTA | |
| | L | ALAVQVVGSNWPKPHYTLLITGLCRFQIVQVLKEKPYPIAE | |
| | | VEQLDRLEEFPNTCKMREELGELSEQFYKYAVQLVEMLDMS | |
| | | VPAVAKLRRLLDSLPREALPDILTSIIRTSNKEKLQILDAV | |
| | | SLEERFKMTIPLLVRQIEGLKLLQKTRKPKQDDDKRVIAIR | |
| | | PIRRITHISGTLEDEDEDEDNDDIVMLEKKIRTSSMPEQAH | |
| | | KVCVKEIKRLKKMPQSMPEYALTRNYLELMVELPWNKSTTD | |
| | | RLDIRAARILLDNDHYAMEKLKKRVLEYLAVRQLKNNLKGP | |
| | | ILCFVGPPGVGKTSVGRSVAKTLGREFHRIALGGVCDQSDI | |
| | | RGHRRTYVGSMPGRIINGLKTVGVNNPVFLLDEVDKLGKSL | |
| | | QGDPAAALLEVLDPEQNHNFTDHYLNVAFDLSQVLFIATAN | |
| | | TTATIPAALLDRMEIIQVPGYTQEEKIEIAHRHLIPKQLEQ | |
| | | HGLTPQQIQIPQVTTLDIITRYTREAGVRSLDRKLGAICRA | |
| | | VAVKVAEGQHKEAKLDRSDVTEREGCREHILEDEKPESISD | |
| | | TTDLALPPEMPILIDFHALKDILGPPMYEMEVSQRLSQPGV | |
| | | AIGLAWTPLGGEIMFVEASRMDGEGQLTLTGQLGDVMKESA | |
| | | HLAISWLRSNAKKYQLTNAFGSFDLLDNTDIHLHFPAGAVT | |
| | | KDGPSAGVTIVTCLASLFSGRLVRSDVAMTGEITLRGLVLP | |
| | | VGGIKDKVLAAHRAGLKQVIIPRRNEKDLEGIPGNVRQDLS | |
| | | FVTASCLDEVLNAAFDGGFTVKTRPGLLNSKLGRKYQKGLN | |
| | | RQQANMLPPTERVLGWQTDGCLIFCETEVLNTGQKMFDCHF | |
| | | NTWK | |
| PEP19- | QQANML | MSSVSPIQIPSRLPLLLTHEGVLLPGSTMRTSVDSARNLQL | 50 |
| 4 | PPTERV | VRSRLLKGTSLQSTILGVIPNTPDPASDAQDLPPLHRIGTA | |
| | L | ALAVQVVGSNWPKPHYTLLITGLCRFQIVQVLKEKPYPIAE | |
| | | VEQLDRLEEFPNTCKMREELGELSEQFYKYAVQLVEMLDMS | |
| | | VPAVAKLRRLLDSLPREALPDILTSIIRTSNKEKLQILDAV | |
| | | SLEERFKMTIPLLVRQIEGLKLLQKTRKPKQDDDKRVIAIR | |
| | | PIRRITHISGTLEDEDEDEDNDDIVMLEKKIRTSSMPEQAH | |
| | | KVCVKEIKRLKKMPQSMPEYALTRNYLELMVELPWNKSTTD | |
| | | RLDIRAARILLDNDHYAMEKLKKRVLEYLAVRQLKNNLKGP | |
| | | ILCFVGPPGVGKTSVGRSVAKTLGREFHRIALGGVCDQSDI | |
| | | RGHRRTYVGSMPGRIINGLKTVGVNNPVFLLDEVDKLGKSL | |
| | | QGDPAAALLEVLDPEQNHNFTDHYLNVAFDLSQVLFIATAN | |
| | | TTATIPAALLDRMEIIQVPGYTQEEKIEIAHRHLIPKQLEQ | |
| | | HGLTPQQIQIPQVTTLDIITRYTREAGVRSLDRKLGAICRA | |
| | | VAVKVAEGQHKEAKLDRSDVTEREGCREHILEDEKPESISD | |
| | | TTDLALPPEMPILIDFHALKDILGPPMYEMEVSQRLSQPGV | |
| | | AIGLAWTPLGGEIMFVEASRMDGEGQLTLTGQLGDVMKESA | |
| | | HLAISWLRSNAKKYQLTNAFGSFDLLDNTDIHLHFPAGAVT | |
| | | KDGPSAGVTIVTCLASLFSGRLVRSDVAMTGEITLRGLVLP | |
| | | VGGIKDKVLAAHRAGLKQVIIPRRNEKDLEGIPGNVRQDLS | |
| | | FVTASCLDEVLNAAFDGGFTVKTRPGLLNSKLGRKYQKGLN | |
| | | RQQANMLPPTERVLGWQTDGCLIFCETEVLNTGQKMFDCHF | |
| | | NTWK | |
| PEP19- | QQANML | MSSVSPIQIPSRLPLLLTHEGVLLPGSTMRTSVDSARNLQL | 50 |
| 5 | PPTERV | VRSRLLKGTSLQSTILGVIPNTPDPASDAQDLPPLHRIGTA | |
| | | ALAVQVVGSNWPKPHYTLLITGLCRFQIVQVLKEKPYPIAE | |
| | | VEQLDRLEEFPNTCKMREELGELSEQFYKYAVQLVEMLDMS | |
| | | VPAVAKLRRLLDSLPREALPDILTSIIRTSNKEKLQILDAV | |
| | | SLEERFKMTIPLLVRQIEGLKLLQKTRKPKQDDDKRVIAIR | |
| | | PIRRITHISGTLEDEDEDEDNDDIVMLEKKIRTSSMPEQAH | |
| | | KVCVKEIKRLKKMPQSMPEYALTRNYLELMVELPWNKSTTD | |
| | | RLDIRAARILLDNDHYAMEKLKKRVLEYLAVRQLKNNLKGP | |
| | | ILCFVGPPGVGKTSVGRSVAKTLGREFHRIALGGVCDQSDI | |
| | | RGHRRTYVGSMPGRIINGLKTVGVNNPVFLLDEVDKLGKSL | |
| | | QGDPAAALLEVLDPEQNHNFTDHYLNVAFDLSQVLFIATAN | |
| | | TTATIPAALLDRMEIIQVPGYTQEEKIEIAHRHLIPKQLEQ | |
| | | HGLTPQQIQIPQVTTLDIITRYTREAGVRSLDRKLGAICRA | |
| | | VAVKVAEGQHKEAKLDRSDVTEREGCREHILEDEKPESISD | |
| | | TTDLALPPEMPILIDFHALKDILGPPMYEMEVSQRLSQPGV | |
| | | AIGLAWTPLGGEIMFVEASRMDGEGQLTLTGQLGDVMKESA | |
| | | HLAISWLRSNAKKYQLTNAFGSFDLLDNTDIHLHFPAGAVT | |
| | | KDGPSAGVTIVTCLASLFSGRLVRSDVAMTGEITLRGLVLP | |
| | | VGGIKDKVLAAHRAGLKQVIIPRRNEKDLEGIPGNVRQDLS | |
| | | FVTASCLDEVLNAAFDGGFTVKTRPGLLNSKLGRKYQKGLN | |
| | | RQQANMLPPTERVLGWQTDGCLIFCETEVLNTGQKMFDCHF | |
| | | NTWK | |
| PEP19- | QQANML | MSSVSPIQIPSRLPLLLTHEGVLLPGSTMRTSVDSARNLQL | |
| 6 | PPTERV | VRSRLLKGTSLQSTILGVIPNTPDPASDAQDLPPLHRIGTA | |
| | | ALAVQVVGSNWPKPHYTLLITGLCRFQIVQVLKEKPYPIAE | |
| | | VEQLDRLEEFPNTCKMREELGELSEQFYKYAVQLVEMLDMS | |
| | | VPAVAKLRRLLDSLPREALPDILTSIIRTSNKEKLQILDAV | |
| | | SLEERFKMTIPLLVRQIEGLKLLQKTRKPKQDDDKRVIAIR | |
| | | PIRRITHISGTLEDEDEDEDNDDIVMLEKKIRTSSMPEQAH | |
| | | KVCVKEIKRLKKMPQSMPEYALTRNYLELMVELPWNKSTTD | |
| | | RLDIRAARILLDNDHYAMEKLKKRVLEYLAVRQLKNNLKGP | |
| | | ILCFVGPPGVGKTSVGRSVAKTLGREFHRIALGGVCDQSDI | |
| | | RGHRRTYVGSMPGRIINGLKTVGVNNPVFLLDEVDKLGKSL | |
| | | QGDPAAALLEVLDPEQNHNFTDHYLNVAFDLSQVLFIATAN | |
| | | TTATIPAALLDRMEIIQVPGYTQEEKIEIAHRHLIPKQLEQ | |
| | | HGLTPQQIQIPQVTTLDIITRYTREAGVRSLDRKLGAICRA | |
| | | VAVKVAEGQHKEAKLDRSDVTEREGCREHILEDEKPESISD | |
| | | TTDLALPPEMPILIDFHALKDILGPPMYEMEVSQRLSQPGV | |
| | | AIGLAWTPLGGEIMFVEASRMDGEGQLTLTGQLGDVMKESA | |
| | | HLAISWLRSNAKKYQLTNAFGSFDLLDNTDIHLHFPAGAVT | |
| | | KDGPSAGVTIVTCLASLFSGRLVRSDVAMTGEITLRGLVLP | |
| | | VGGIKDKVLAAHRAGLKQVIIPRRNEKDLEGIPGNVRQDLS | |
| | | FVTASCLDEVLNAAFDGGFTVKTRPGLLNSKLGRKYQKGLN | |
| | | RQQANMLPPTERVLGWQTDGCLIFCETEVLNTGQKMFDCHF | |
| | | NTWK | |
| PEP19- | QQANML | MSSVSPIQIPSRLPLLLTHEGVLLPGSTMRTSVDSARNLQL | 50 |
| 6 | PPTERV | VRSRLLKGTSLQSTILGVIPNTPDPASDAQDLPPLHRIGTA | |
| | L | ALAVQVVGSNWPKPHYTLLITGLCRFQIVQVLKEKPYPIAE | |
| | | VEQLDRLEEFPNTCKMREELGELSEQFYKYAVQLVEMLDMS | |
| | | VPAVAKLRRLLDSLPREALPDILTSIIRTSNKEKLQILDAV | |
| | | SLEERFKMTIPLLVRQIEGLKLLQKTRKPKQDDDKRVIAIR | |
| | | PIRRITHISGTLEDEDEDEDNDDIVMLEKKIRTSSMPEQAH | |
| | | KVCVKEIKRLKKMPQSMPEYALTRNYLELMVELPWNKSTTD | |
| | | RLDIRAARILLDNDHYAMEKLKKRVLEYLAVRQLKNNLKGP | |
| | | ILCFVGPPGVGKTSVGRSVAKTLGREFHRIALGGVCDQSDI | |
| | | RGHRRTYVGSMPGRIINGLKTVGVNNPVFLLDEVDKLGKSL | |
| | | QGDPAAALLEVLDPEQNHNFTDHYLNVAFDLSQVLFIATAN | |
| | | TTATIPAALLDRMEIIQVPGYTQEEKIEIAHRHLIPKQLEQ | |
| | | HGLTPQQIQIPQVTTLDIITRYTREAGVRSLDRKLGAICRA | |
| | | VAVKVAEGQHKEAKLDRSDVTEREGCREHILEDEKPESISD | |
| | | TTDLALPPEMPILIDFHALKDILGPPMYEMEVSQRLSQPGV | |
| | | AIGLAWTPLGGEIMFVEASRMDGEGQLTLTGQLGDVMKESA | |
| | | HLAISWLRSNAKKYQLTNAFGSFDLLDNTDIHLHFPAGAVT | |
| | | KDGPSAGVTIVTCLASLFSGRLVRSDVAMTGEITLRGLVLP | |
| | | VGGIKDKVLAAHRAGLKQVIIPRRNEKDLEGIPGNVRQDLS | |
| | | FVTASCLDEVLNAAFDGGFTVKTRPGLLNSKLGRKYQKGLN | |
| | | RQQANMLPPTERVLGWQTDGCLIFCETEVLNTGQKMFDCHF | |
| | | NTWK | |
| PEP20- | RASLCG | MARASLCGKEHTPEMWTRPPQEGPCLEVEINENLPARKT | 51 |
| 1 | K | | |
| PEP20- | RASLCG | MARASLCGKEHTPEMWTRPPQEGPCLEVEINENLPARKT | 51 |
| 2 | K | | |
| PEP20- | RASLCG | MARASLCGKEHTPEMWTRPPQEGPCLEVEINENLPARKT | 51 |
| 3 | K | | |
| PEP20- | RASLCG | MARASLCGKEHTPEMWTRPPQEGPCLEVEINENLPARKT | 51 |
| 4 | K | | |
| PEP21- | RLSQLP | MEAWRGYVLIHGYTARKWKSWDPKPTHLTRTRPWAWQRQLP | 52 |
| 1 | LK | DTPPYSASDSCSPPQVKGECDPPSAHLALLLFLLDSGPCSC | |
| | | DAAHAPAAEHLWNGRLLPNPRRLSQLPLKRQSSCHPPGPIR | |
| | | VLPSGDRLFLPSAASVSQPWSLIASNQEEKVHAGTGGLRGM | |
| | | PSVGLPLQTDDK | |
| PEP21- | RLSQLP | MDVVGENEALQQFFEAQGANGTLENPALDTSLLEEFLGNDF | 53 |
| 2 | LK | DLGAFCSCDAAHAPAAEHLWNGRLLPNPRRLSQLPLKRQSS | |
| | | CHPPGPIRVLPSGDRLFLPSAASVSQPWSLIASNQEEKVHA | |
| | | GTGGLRGMPSVGLPLQTDDK | |
| PEP21- | RLSQLP | MEAWRGYVLIHGYTARKWKSWDPKPTHLTRTRPWACCSCDA | 54 |
| 3 | LK | AHAPAAEHLWNGRLLPNPRRLSQLPLKRQSSCHPPGPIRVL | |
| | | PSGDRLFLPSAASVSQPWSLIASNQEEKVHAGTGGLRGMPS | |
| | | VGLPLQTDDK | |
| PEP21- | RLSQLP | MEAWRGYVLIHGYTARKWKSWDPKPTHLTRTRPWAWCMLPN | 55 |
| 4 | LK | PEAHSWEDSSSFSPPHSCSCDAAHAPAAEHLWNGRLLPNPR | |
| | | RLSQLPLKRQSSCHPPGPIRVLPSGDRLFLPSAASVSQPWS | |
| | | LIASNQEEKVHAGTGGLRGMPSVGLPLQTDDK | |
| PEP22- | SAQTGL | MAMQKIFAREILDSRGNPTVEVDLHTAKGRFRAAVPSGAST | 56 |
| 1 | S | GIYEALELRDGDKGRYLGKGVLKAVENINNTLGPALLQKAS | |
| | | GEARSLQPPPHAPAPSAQTGLSRNIFPYPSPACALTSEKSD | |
| | | LCSPFSNSPFQKLSVVDQEKVDKFMIELDGTENKSKFGANA | |
| | | ILGVSLAVCKAGAAEKGVPLYRHIADLAGNPDLILPVPAFN | |
| | | VINGGSHAGNKLAMQEFMILPVGASSFKEAMRIGAEVYHHL | |
| | | KGVIKAKYGKDATNVGDEGGFAPNILENNEALELLKTAIQA | |
| | | AGYPDKVVIGMDVAASEFYRNGKYDLDFKSPDDPARHITGE | |
| | | KLGELYKSFIKNYPGEAFGCPSVPARIPCSCLIY | |
| PEP22- | SAQTGL | MAMQKIFAREILDSRGNPTVEVDLHTAKGRFRAAVPSGAST | 56 |
| 2 | S | GIYEALELRDGDKGRYLGKGVLKAVENINNTLGPALLQKAS | |
| | | GEARSLOPPPHAPAPSAQTGLSRNIFPYPSPACALTSEKSD | |
| | | LCSPFSNSPFQKLSVVDQEKVDKFMIELDGTENKSKFGANA | |
| | | ILGVSLAVCKAGAAEKGVPLYRHIADLAGNPDLILPVPAFN | |
| | | VINGGSHAGNKLAMQEFMILPVGASSFKEAMRIGAEVYHHL | |
| | | KGVIKAKYGKDATNVGDEGGFAPNILENNEALELLKTAIQA | |
| | | AGYPDKVVIGMDVAASEFYRNGKYDLDFKSPDDPARHITGE | |
| | | KLGELYKSFIKNYPGEAFGCPSVPARIPCSCLIY | |
| PEP22- | SAQTGL | MAMQKIFAREILDSRGNPTVEVDLHTAKGRFRAAVPSGAST | 56 |
| 3 | S | GIYEALELRDGDKGRYLGKGVLKAVENINNTLGPALLQKAS | |
| | | GEARSLQPPPHAPAPSAQTGLSRNIFPYPSPACALTSEKSD | |
| | | LCSPFSNSPFQKLSVVDQEKVDKFMIELDGTENKSKFGANA | |
| | | ILGVSLAVCKAGAAEKGVPLYRHIADLAGNPDLILPVPAFN | |
| | | VINGGSHAGNKLAMQEFMILPVGASSFKEAMRIGAEVYHHL | |
| | | KGVIKAKYGKDATNVGDEGGFAPNILENNEALELLKTAIQA | |
| | | AGYPDKVVIGMDVAASEFYRNGKYDLDFKSPDDPARHITGE | |
| | | KLGELYKSFIKNYPGEAFGCPSVPARIPCSCLIY | |
| PEP22- | SAQTGL | MAMQKIFAREILDSRGNPTVEVDLHTAKGRFRAAVPSGAST | 56 |
| 4 | S | GIYEALELRDGDKGRYLGKGVLKAVENINNTLGPALLQKAS | |
| | | GEARSLOPPPHAPAPSAQTGLSRNIFPYPSPACALTSEKSD | |
| | | LCSPFSNSPFQKLSVVDQEKVDKFMIELDGTENKSKFGANA | |
| | | ILGVSLAVCKAGAAEKGVPLYRHIADLAGNPDLILPVPAFN | |
| | | VINGGSHAGNKLAMQEFMILPVGASSFKEAMRIGAEVYHHL | |
| | | KGVIKAKYGKDATNVGDEGGFAPNILENNEALELLKTAIQA | |
| | | AGYPDKVVIGMDVAASEFYRNGKYDLDFKSPDDPARHITGE | |
| | | KLGELYKSFIKNYPGEAFGCPSVPARIPCSCLIY | |
| PEP23 | SGSEEV | MTTAGRGNLGLIPRSTAFQKQEGRLTVKQEPANQTWGQGSS | 57 |
| | R | LQKNYPPVCEIFRLHFRQLCYHEMSGPQEALSRLRELCRWW | |
| | | LMPEVHTKEQILELLVLEQFLSILPGELRTWVQLHHPESGE | |
| | | EAVAVVEDFQRHLSGSEEVRT | |
| PEP24 | SPDSTL | MSQRAKLRSRENQPTVFLPSPDSTLRKYYGEKIGIYFAWLG | 58 |
| | R | YYTQMLLLAAVVGVACFLYGYLNQDNCTWSKEVCHPDIGGK | |
| | | IIMCPQCDRLCPFWKLNITCESSKKLCIFDSFGTLVFAVFM | |
| | | GVWVTLFLEFWKRRQAELEYEWDTVELQQEEQARPEYEARC | |
| | | THVVINEITQEEERIPFTAWGKCIRITLCASAVFFWILLII | |
| | | ASVIGIIVYRLSVFIVESAKLPKNINGTDPIQKYLTPQTAT | |
| | | SITASIISFIIIMILNTIYEKVAIMITNFELPRTQTDYENS | |
| | | LTMKMFLFQFVNYYSSCFYIAFFKGKFVGYPGDPVYWLGKY | |
| | | RNEECDPGGCLLELTTOLTIIMGGKAIWNNIQEVLLPWIMN | |
| | | LIGRFHRVSGSEKITPRWEQDYHLQPMGKLGLFYEYLEMII | |
| | | QFGFVTLFVASFPLAPLLALVNNILEIRVDAWKLTTQFRRL | |
| | | VPEKAQDIGAWQPIMQGIAILAVVTNAMIIAFTSDMIPRLV | |
| | | YYWSFSVPPYGDHTSYTMEGYINNTLSIFKVADFKNKSKGN | |
| | | PYSDLGNHTTCRYRDFRYPPGHPQEYKHNIYYWHVIAAKLA | |
| | | FIIVMEHVIYSVKFFISYAIPDVSKRTKSKIQREKYLTQKL | |
| | | LHENHLKDMTKNMGVIAERMIEAVDNNLRPKSE | |
| PEP25 | SPGYGS | MAERRAFAQKISRTVAAEVRKQISGQYSGSPQLLKNLNIVG | 59 |
| | K | NISHHTTVPLTEAVDPVDLEDYLITHPLAVDSGPLRDLIEF | |
| | | PPDDIEVVYSPRDCRTLVSAVPEESEMDPHVRDCIRSYTED | |
| | | WAIVIRKYHKLGTGFNPNTLDKQKERQKGLPKQVFESDEAP | |
| | | DGNSYQDDQDDLKRRSMSIDDTPRGSWACSIFDLKNSLPDA | |
| | | LLPNLLDRTPNEEIDRONDDQRKSNRHKELFALHPSPDEEE | |
| | | PIERLSVPDIPKEHFGQRLLVKCLSLKFEIEIEPIFASLAL | |
| | | YDVKEKKKISENFYFDLNSEQMKGLLRPHVPPAAITTLARS | |
| | | AIFSITYPSQDVFLVIKLEKVLQQGDIGECAEPYMIFKEAD | |
| | | ATKNKEKLEKLKSQADQFCQRLGKYRMPFAWTAIHLMNIVS | |
| | | SAGSLERDSTEVEISTGERKGSWSERRNSSIVGRRSLERTT | |
| | | SGDDACNLTSFRPATLTVTNFFKQEGDRLSDEDLYKFLADM | |
| | | RRPSSVLRRLRPITAQLKIDISPAPENPHYCLTPELLQVKL | |
| | | YPDSRVRPTREILEFPARDVYVPNTTYRNLLYTYPQSLNFA | |
| | | NRQGSARNITVKVQFMYGEDPSNAMPVIFGKSSCSEFSKEA | |
| | | YTAVVYHNRSPDFHEEIKVKLPATLTDHHHLLFTFYHVSCQ | |
| | | QKQNTPLETPVGYTWIPMLQNGRLKTGQFCLPVSLEKPPQA | |
| | | YSVLSPEVPLPGMKWVDNHKGVFNVEVVAVSSIHTQDPYLD | |
| | | KFFALVNALDEHLFPVRIGDMRIMENNLENELKSSISALNS | |
| | | SQLEPVVRFLHLLLDKLILLVIRPPVIAGQIVNLGQASFEA | |
| | | MASIINRLHKNLEGNHDQHGRNSLLASYIHYVFRLPNTYPN | |
| | | SSSPGYGSKL | |
| PEP26 | SSGLGL | MAPRGRKRKAEAAVVAVAEKREKLANGGEGMEEATVVIEHC | 60 |
| | RR | TSVRSSGLGLRRGPHANSNSLSLKRWWKS | |
| PEP27 | VWGAGR | MQRCPGPLGRGDPPSRKLGLVSVPLQPQGLARMLGAPHPGD | 61 |
| | R | SAHQGLRGGGSPGTWEAGPPAPWTPTQTPSQPRHFPRARGQ | |
| | | PGSPGLREGRVWGAGRRHIPLLMMPPQSYNLDSRRSCPFPP | |
| | | SPVPGGSPDPFREDHGP | |

The RNA-seq data can be obtained from a healthy cell from a patient or a healthy donor and/or a diseased cell such as tumor tissue from the same patient or a different patient. The cells used to obtain RNA-seq data can also include cell lines, such as commercially available cell lines, cell lines derived from patients, and cell lines derived from organoids derived from patient samples. The RNA-seq data can be analyzed for alternative splicing events by using a computer implemented method that can quantify and analyze alternative splicing events and generates exon duos or exon trios comprising the alternative splicing junctions. One or more datasets of RNA-seq data can be compared for alternative splicing events presence or absence.

The cell surface antigen can be derived from different types of alternative splicing for example intron retention, frameshift, translated lncRNA, novel splicing junction, novel exon, or chimeric neoantigens.

In certain embodiments, the cell surface antigen isoform has a transmembrane domain, whereas the major isoform has no transmembrane domain. In certain embodiments, the cell surface antigen isoform has no transmembrane domain, whereas the major splicing isoform has a transmembrane domain. Other examples of membrane topology can comprise residence of the cell surface antigen isoform in intracellular or extracellular compartment, or novel topology in the membrane, i.e., one, two, three, four or more novel transmembrane regions. In certain embodiments the cell surface antigen isoform gains a transmembrane region compared to major splicing isoform. In certain embodiments the cell surface antigen isoform has a transmembrane region less compared to the major splicing isoform.

In certain embodiments, a set of cell surface antigen derived peptides can be selected wherein the peptides have an increased likelihood of being presented on the tumor cell surface relative to unselected peptides. The cell surface presentation of the cell surface antigen derived peptide can be MHC-dependent or MHC-independent. In some embodiments the cell surface antigen is MHC I dependent.

Ranking can be performed using the plurality of cell surface antigens provided by at least one model based at least in part on the numerical likelihoods. Following the ranking a selection can be performed to select a subset of the ranked cell surface antigens according to a selection criteria for example membrane topology, B cell antibody accessibility, or T cell antigenicity. After selecting a subset of the ranked peptides can be provided as an output. A number of the set of selected cell surface antigens may be 1, 2, 3, 4, 5, 6, 7, 8, 9, 10 or more cell surface antigens.

In certain embodiments, the diseased cell is a cancer cell. The cancer can be for example a bone cancer, a breast cancer, a colorectal cancer, a gastric cancer, a liver cancer, a lung cancer, an ovarian cancer, a pancreatic cancer, a prostate cancer, a skin cancer, a testicular cancer, a blood cancer, brain cancer, and a vaginal cancer. In certain embodiments, the blood cancer is a leukemia, a non-Hodgkin lymphoma, a Hodgkin lymphoma, or a multiple myeloma. In certain embodiments the cancer is a blood cancer, such as Acute Myeloid Leukemia (AML). Other exemplary cancers with a high alternative splicing burden comprise but are not limited to triple-negative breast cancer (TNBC), non-small cell lung carcinoma (NSCLC), Kidney Renal Clear Cell Carcinoma (KIRC), Lung Adenocarcinoma (LUAD), Ovarian Cancer (OV), Breast Invasive Carcinoma (BRCA), and Uterine Corpus Endometrial Carcinoma (UCEC). In some embodiments the diseased cell is from other diseases with a high alternative splicing burden including autoimmune disorders, such as Type 1 diabetes, multiple sclerosis, and rheumatoid arthritis, among others.

TABLE 3 shows exemplary types of cancer with a high alternative splicing burden and exemplary cell surface antigens identified in EXAMPLE 3.

TABLE 3

| | CPTAC | |
|---|---|---|
| Type of Cancer | Cancer Type | PEP ID NO: |
| Kidney Renal Clear Cell | KIRC | 1, 27, 8, 11, 16, 20, 24, 26, |
| Carcinoma | | 3, 9 |
| Lung Adenocarcinoma | LUAD | 1, 10, 13, 15, 22, 8, 12, 16, |
| | | 18, 2, 20, 23, 25, 4, 6, 9 |
| Ovarian Cancer | OV | 14, 7 |
| Breast Invasive Carcinoma | BRCA | 17, 21, 5 |
| Uterine Corpus Endometrial | UCEC | 27 |
| Carcinoma | | |
| Gastrointestinal cancer | GI | 19 |

In certain embodiments the method also comprises generating an output for constructing a personalized cancer vaccine from the selected cell surface antigens. In certain embodiments the personalized cancer vaccine comprises at least one cell surface antigen sequence or at least one nucleotide sequence encoding the selected cell surface antigen or fragments thereof.

In certain embodiments the method also comprises obtaining an antibody or ADC that specifically binds the selected cell surface antigens. In other embodiments, the method comprises obtaining a therapeutic for example Tumor Infiltrating Lymphocytes (TILs) specific for a cell surface antigen, T cell Receptor (TCR) engineered T cells specific for a cell surface antigen, Antibodies, Fabs, scFvs, Bi and Trispecific cell engagers specific for a cell surface antigen, or CAR-T cells specific for a cell surface antigen and administering the therapeutic to the subject in need of treatment.

In another aspect, disclosed herein are computer implemented systems identifying one or more cell surface antigens resulting from alternative splicing in a cell. As an example, one such system may comprise a digital processing device comprising a processor, an operating system configured to perform executable instructions, a memory, and a computer program including instructions executable by the digital processing device to create an cell surface antigen analysis application, the application comprising a software module for: a digital processing device comprising a processor, an operating system configured to perform executable instructions, a memory, and a computer program including instructions executable by the digital processing device to create an cell surface antigen analysis application, the application comprising a software module for: (a) obtaining a first RNA-seq data set from a first sample cell and a second RNA-seq data set from a second sample cell; (b) assembling full length mRNA transcript sequences and extracting genomic loci coordinates of the mRNA transcript sequences; (c) clustering of full length mRNA transcript sequences encoded at the same genomic loci and extraction of exon duo or exon trio mRNA sequences; (d) selecting the most representative full length mRNA transcript sequences; (e) identifying stable full length mRNAs transcripts; (f) translating, in silico the stable full length mRNA transcripts into protein isoform sequences; (g) identifying protein isoform sequences that are predicted to be stable; (h) determining B cell antibody accessibility of the protein isoform sequences by using an algorithm to classify the polarity, hydrophobicity, and surface accessibility of peptides derived from the protein isoform sequences; (i) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; (j) generating a first set of antigenic cell surface antigen sequences based on the first RNA-seq data set and a second set of antigenic cell surface antigen sequences based on the second RNA-seq data set ranked by B cell antibody accessibility and T cell antigenicity; and (k) determining unique antigenic cell surface antigen sequences by comparing the first set of antigenic cell surface antigen sequences and the second set of antigenic cell surface antigen sequences and selecting cell surface antigen sequences present in one set and not the other set; thereby selecting one or more unique cell surface antigen sequences.

In another embodiment, the system comprises a digital processing device comprising a processor, an operating system configured to perform executable instructions, a memory, and a computer program including instructions executable by the digital processing device to create an cell surface antigen analysis application, the application comprising a software module for:

(a) obtaining a first RNA-seq data set from a first sample cell and a second RNA-seq data set from a second sample cell; (b) assembling full length mRNA transcript sequences and extracting genomic loci coordinates of the mRNA transcript sequences; (c) clustering of full length mRNA transcript sequences encoded at the same genomic loci and extraction of exon duo or exon trio mRNA sequences; (d) selecting the most representative full length mRNA transcript sequences; (e) identifying stable full length mRNAs transcripts; (f) translating, in silico the stable full length mRNA transcripts into protein isoform sequences; (g) identifying protein isoform sequences that are predicted to be stable; (h) determining membrane topologies for each protein isoform; (i) filtering for membrane bound protein isoform sequences; (j) determining B cell antibody accessibility of the protein isoform sequences by using an algorithm to classify the polarity, hydrophobicity, and surface accessibility of peptides derived from the protein isoform sequences; (k) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; (l) generating a first set of antigenic cell surface antigen sequences based on the first RNA-seq data set and a second set of antigenic cell surface antigen sequences based on the second RNA-seq data set ranked by B cell antibody accessibility and T cell antigenicity; and (m) determining unique antigenic cell surface antigen sequences by comparing the first set of antigenic cell surface antigen sequences and the second set of antigenic cell surface antigen sequences and selecting cell surface antigen sequences present in one set and not the other set; selecting one or more unique cell surface antigen sequences.

### IV. Pharmaceutical Compositions

As described above, the disclosed methods can involve selecting and validating an intervention, which can include a therapeutic. In various embodiments, the intervention includes a pharmaceutical composition including the therapeutic.

### Pharmaceutical Compositions

In various embodiments, the pharmaceutical compound includes an acceptable pharmaceutically acceptable carrier. The carrier(s) should be “acceptable” in the sense of being compatible with the other ingredients of the formulations and not deleterious to the subject. Pharmaceutically acceptable carriers include buffers, solvents, dispersion media, coatings, isotonic and absorption delaying agents, and the like, that are compatible with pharmaceutical administration. In one embodiment the pharmaceutical composition is administered orally and includes an enteric coating suitable for regulating the site of absorption of the encapsulated substances within the digestive system or gut.

Pharmaceutical compositions containing a therapeutic, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Such pharmaceutically acceptable carriers can be sterile liquids, such as water and oil, including those of petroleum, animal, vegetable or synthetic origin, such as peanut oil, soybean oil, mineral oil, and the like. Saline solutions and aqueous dextrose, polyethylene glycol (PEG) and glycerol solutions can also be employed as liquid carriers, particularly for injectable solutions. The pharmaceutical composition may further comprise additional ingredients, for example preservatives, buffers, tonicity agents, antioxidants and stabilizers, nonionic wetting or clarifying agents, viscosity increasing agents, and the like. The pharmaceutical compositions described herein can be packaged in single unit dosages or in multidosage forms. The compositions are generally formulated as sterile and substantially isotonic solution.

In one embodiment the cell surface antigen derived peptide, vaccine, antibody, bispecific cell engager, trispecific cell engager, ADC, CAR-T cell, or TCR engineered T cell for use in the target cells as detailed above is formulated into a pharmaceutical composition intended for oral, inhalation, intranasal, intratracheal, intravenous, intramuscular, subcutaneous, intradermal, and other parental routes of administration. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322. In one embodiment, the carrier is an isotonic sodium chloride solution. In another embodiment, the carrier is balanced salt solution. In one embodiment, the carrier includes tween. In another embodiment, the pharmaceutically acceptable carrier comprises a surfactant, such as perfluorooctane (Perfluoron liquid). Routes of administration may be combined, if desired.

In another aspect, disclosed herein are methods for treating subjects having a cancer. In some embodiments, the method comprises the steps of identifying one or more cell surface antigens and cell surface antigen derived peptides resulting from alternative splicing in a cell, comprising the steps of: (a) obtaining a first RNA-seq data set from a first sample cell and a second RNA-seq data set from a second sample cell; (b) assembling full length mRNA transcript sequences and extracting genomic loci coordinates of the mRNA transcript sequences; (c) clustering of full length mRNA transcript sequences encoded at the same genomic loci and extraction of exon duo or exon trio mRNA sequences; (d) selecting the most representative full length mRNA transcript sequences; (e) identifying stable full length mRNAs transcripts; (f) translating, in silico the stable full length mRNA transcripts into protein isoform sequences; (g) identifying protein isoform sequences that are predicted to be stable; (h) determining B cell antibody accessibility of the protein isoform sequences by using an algorithm to classify the polarity, hydrophobicity, and surface accessibility of peptides derived from the protein isoform sequences; (i) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; (j) generating a first set of antigenic cell surface antigen sequences based on the first RNA-seq data set and a second set of antigenic cell surface antigen sequences based on the second RNA-seq data set ranked by B cell antibody accessibility and T cell antigenicity; and (k) determining unique antigenic cell surface antigen sequences by comparing the first set of antigenic cell surface antigen sequences and the second set of antigenic cell surface antigen sequences and selecting cell surface antigen sequences present in one set and not the other set; thereby selecting one or more unique cell surface antigen sequences, and obtaining a cancer vaccine comprising one or more selected cell surface antigens or antigenic peptide derived from the cell surface antigen, and administering the cancer vaccine to the subject. In some embodiments, the method further comprises determining membrane topologies for each protein isoform sequence and filtering for membrane bound protein isoform sequences.

In another aspect, disclosed herein are compositions for treating subjects having a cancer. In some embodiments, the composition comprises an isolated peptide comprising a cell surface antigen or a peptide derived thereof comprising a sequence set forth in TABLE 1, wherein the peptide is no more than 100 amino acids in length, and an optional pharmaceutically acceptable carrier. In some embodiments the isolated peptide is no more than 30 amino acids in length or 20 amino acids in length. In some embodiments the amino acid sequence of the peptide consists essentially of or consists of an amino acid sequence set forth in TABLE 1. In some embodiments the isolated peptide comprises an amino acid sequence set forth in TABLE 1 and is presentable by a major histocompatibility complex (MHC) Class I or MHC Class II. In some embodiments the isolated peptide is synthetic.

In some embodiments, a pharmaceutical composition is provided. For example, the pharmaceutical composition can comprise an isolated peptide comprising a cell surface antigen or a peptide derived thereof comprising a sequence set forth in TABLE 1 or TABLE 2, wherein the peptide is no more than 100 amino acids in length, and pharmaceutically acceptable carrier or excipient. In some embodiments the isolated peptide is no more than 30 amino acids in length or 20 amino acids in length. In some embodiments the amino acid sequence of the peptide consists essentially of or consists of an amino acid sequence set forth in TABLE 1. In some embodiments, the isolated peptide comprises an amino acid sequence set forth in TABLE 1 and is presentable by a major histocompatibility complex (MHC) Class I or MHC Class II. In some embodiments, the isolated peptide is synthetic. In some embodiments, the pharmaceutical composition comprises a plurality of peptides (e.g., 2, 3, 4, 5, 6, 7, 8, 9, 10, or more) set forth in TABLE 1 and a pharmaceutically acceptable carrier or excipient. The pharmaceutical composition can additionally or alternatively comprise a nucleic acid encoding a peptide set forth in TABLE 1 and a pharmaceutically acceptable carrier or excipient. In some embodiments the pharmaceutical composition further comprises a liposome or a lipid nanoparticle.

In some embodiments the pharmaceutical compositions described herein comprise human, mouse, chimeric or humanized antibodies, ADCs, bispecific cell engagers, or trispecific cell engagers. Antibodies can be raised against any cell surface antigen listed in TABLE 1 or TABLE 2. Antibodies, ADCs, bispecific antibodies and cell engagers, and trispecific antibodies and cell engagers can be formulated into pharmaceutical compositions and administered to a patient in need thereof.

In some embodiments, the pharmaceutical composition can include adoptive cell therapies such as CAR-T cells and TCR engineered T cells. The cell therapies can be formulated into pharmaceutical compositions and administered to a patient in need thereof.

### Vaccines

The cell surface antigens or derived peptides can be used to design prophylactic or therapeutic vaccines comprising such composition (e.g., pharmaceutical compositions) for immunizing subjects having cancer or are at risk for cancer. A vaccine composition of the disclosure can comprise a peptide composition(s) comprising the cell surface antigens or derived peptides. Alternatively, a vaccine composition of the invention can comprise a nucleic acid composition, e.g., an RNA composition or DNA composition, encoding the cell surface antigens or derived peptides. For such nucleic acid vaccines, suitable regulatory sequences are included such that the peptide epitope is expressed from the nucleic acid (RNA or DNA) in cells of the subject being immunized. Candidate vaccine platforms for cancer vaccines include peptides, RNA, DNA, DCs, and viral vectors.

In certain embodiments, the vaccine of the disclosure comprises at least one cancer cell surface antigen or derived peptide such that the vaccine stimulates a T cell immune response when administered to a subject. In various embodiments, the vaccine comprises, e.g., at least one cell surface antigens or derived peptides, e.g., comprising a sequence shown in TABLE 1, and/or combinations thereof. In certain embodiments, the composition comprises two or more (e.g., three or more, four or more, five or more, six or more, seven or more, eight or more, nine or more, ten or more, 11 or more, 12 or more, 13 or more, 14, or more, 15 or more, 16 or more, 17 or more, 18 or more, 19 or more, or 20 or more) of the peptides disclosed herein (e.g., set forth in TABLE 1). In certain embodiments, the two or more peptides are derived from the same cancer cell surface antigen. In certain embodiments, the two or more peptides are derived from at least two different cancer cell surface antigen. Exemplary cancers for treatment with the vaccines of the disclosure are listed in TABLE 3.

In certain embodiments, the two or more peptides collectively are recognized by MHC molecules in at least 50%, at least 60%, at least 70%, at least 80%, at least 90%, at least 95%, or at least 99% of the human population. In certain embodiments, the vaccine contains individualized components according to the personal need (e.g., MHC variants) of the particular patient.

A vaccine composition of the disclosure can comprise one or more short (e.g., 8-35 amino acids) peptides as the immunostimulatory agent. In certain embodiments, a cell surface antigen sequence is incorporated into a larger carrier polypeptide or protein, to create a chimeric carrier polypeptide or protein that comprises the T cell epitope(s). This chimeric carrier polypeptide or protein can then be incorporated into the vaccine composition.

Recombinant cells can be engineered to express proteins and peptides of the disclosure. Vectors can be designed for the expression of cell surface antigens (e.g. nucleic acid transcripts, proteins, or enzymes) in prokaryotic or eukaryotic cells. For example, cell surface antigens can be expressed in bacterial cells such as Escherichia coli, insect cells (using baculovirus expression vectors), yeast cells, or mammalian cells. Suitable host cells are discussed further in Goeddel (1990) Gene Expression Technology: Methods in Enzymology 185, Academic Press, San Diego, Calif. The cell surface antigens can be purified from the recombinant cells and used in antibody development or further formulated into pharmaceutical compositions. Additionally or alternatively, the recombinant cells expressing the cell surface antigens can be used for producing antibodies or T cells specific to the cell surface antigens.

It is understood that a peptide can be expressed from a nucleic acid (e.g., an mRNA) in a cell of the subject. Exemplary methods of producing peptides by translation in vitro or in vivo are described in U.S. Patent Application Publication No. 2012/0157513 and He et al., J. Ind. Microbiol. Biotechnol. (2015) 42(4):647-53. The present disclosure provides a composition (e.g., pharmaceutical composition) comprising one or more nucleic acids (e.g., mRNAs) encoding one or more cell surface antigens or derived peptides. It is understood that a peptide can be expressed from a nucleic acid (e.g., an mRNA) in a cell of the subject. Exemplary methods of producing peptides by translation in vitro or in vivo are described in U.S. Patent Application Publication No. 2012/0157513 and He et al., J. Ind. Microbiol. Biotechnol. (2015) 42(4):647-53. The present disclosure provides a composition (e.g., pharmaceutical composition) comprising one or more nucleic acids (e.g., mRNAs) encoding one or more peptides disclosed herein, optionally further comprising a pharmaceutically acceptable carrier or excipient. In certain embodiments, the composition comprises nucleic acid sequences encoding two or more (e.g., three or more, four or more, five or more, six or more, seven or more, eight or more, nine or more, ten or more, 11 or more, 12 or more, 13 or more, 14, or more, 15 or more, 16 or more, 17 or more, 18 or more, 19 or more, or 20 or more) of the peptides disclosed herein. In certain embodiments, the two or more peptides are derived from the same cell surface antigen. In certain embodiments, the two or more peptides are derived from at least two different cell surface antigens. In certain embodiments, the composition comprises a nucleic acid sequence encoding one or more of the cell surface antigen set forth in TABLE 1. In certain embodiments, the two or more peptides collectively are recognized by MHC molecules in at least 50%, at least 60%, at least 70%, at least 80%, at least 90%, at least 95%, or at least 99% of the human population. In certain embodiments, the vaccine contains individualized components according to the personal need (e.g., MHC variants) of the particular patient. In certain embodiments, each of the nucleic acids further comprises one or more expression control sequences (e.g., promoter, enhancer, translation initiation site, internal ribosomal entry site, and/or ribosomal skipping element) operably linked to one or more of the peptide coding sequences.

In certain embodiments, the composition or vaccine comprises at least one immunogenicity enhancing adjuvant. Adjuvants included in the vaccine preparation are selected to enhance immune responsiveness to the cell surface antigen(s) while maintaining suitable pharmaceutical delivery and avoiding detrimental side effects. Numerous adjuvants and excipients known in the art for use in cell surface antigen vaccines can be evaluated for inclusion in the vaccine composition. Suitable adjuvants include any substance that, for example, activates or accelerates the immune system to cause an enhanced antigen-specific immune response. Examples of adjuvants that can be used in the present invention include mineral salts, such as calcium phosphate, aluminum phosphate and aluminum hydroxide; immunostimulatory DNA or RNA, such as CpG oligonucleotides; proteins, such as antibodies or Toll-like receptor binding proteins; saponins (e.g., QS21); cytokines; muramyl dipeptide derivatives; LPS; MPL and derivatives including 3D-MPL; GM-CSF (Granulocyte-macrophage colony-stimulating factor); imiquimod; colloidal particles; complete or incomplete Freund's adjuvant; Ribi's adjuvant or bacterial toxin e.g. cholera toxin or enterotoxin (LT). Neoantigen cancer vaccines are reviewed in Blass E. et al., Nature Reviews Clinical Oncology (2021) 18:215-229. The amounts and concentrations of adjuvants useful in the context of the present invention can be readily determined by the skilled artisan without undue experimentation.

### V. Methods of Treatment

Described herein are various methods of preventing, treating, arresting progression of or ameliorating disease and disorders as described herein. Generally, the methods include administering to a subject, e.g., a subject, in need thereof, an effective amount of a composition comprising a vaccine, antibody, ADC, bispecific antibody or T cell engager, trispecific antibody or T cell engager, or adoptive cell therapy as described above and a pharmaceutically acceptable carrier. Any of the pharmaceutical compositions described herein are useful in the methods described below.

### Breast Cancer

In some embodiments, any of the treatments and or methods disclosed herein is for use in treatment of a patient having breast cancer.

Breast invasive carcinoma (BRCA) is the most commonly diagnosed cancer and second-leading cause of cancer mortality in women with nearly 30% of primary disease diagnoses turning into metastatic BRCA. The biggest challenge in BRCA treatment is overcoming its large heterogeneity and distinct cancer subtypes that demand differential treatments including chemotherapy, hormonal therapy, and HER2 targeted therapy depending on the subtype. However, a significant number of patients develop resistance to current standard of care therapies; highlighting the need to identify novel targets and develop alternative therapies for complete disease remission. Recently, immune check point inhibitors Tecentriq and Keytruda were approved for BRCA treatment. Total Mutational Burden (TMB) is defined as the total number of somatic coding mutations in a tumor and is used as a biomarker for IO response. It is generally assumed that high TMB is correlated with an increased probability of immunogenic peptide generation. As a result, the applicability of IO is biased towards cancers with high TMB (e.g., melanoma), failing to reach significant patient populations with lower TMB, like acute myeloid leukemia (AML) or breast cancer. Because BRCA falls in the medium-low (TMB) spectrum, it fails to predict patient response, and there is urgent need to identify alternative markers for immunotherapy response and patient stratification, such as splicing aberrations affecting gene function and protein expression. Aberrant splicing is a major source of coding variation in BRCA, which directly results from the overexpression of key regulatory splicing factors in tumors. Many studies have demonstrated the oncogenic role of splicing factors like SRSF1, SRSF2, ESRP1, RBFOX1 and TRA2B in BRCA (for example, described in Read A. et al., Endocr Relat Cancer. (2018) (9):R467-78, Chan S. et al., Mol Cancer Ther. (2017) 16(12):2849, Drost J. et al., Development (2017) 144(6):968, Dutta D. et al., Trends Mol Med. (2017) (5):393-410, and Aboulkheyr Es H. et al., Trends Biotechnol. (2018) 36(4):358-71), and numerous splicing events have been linked to tumor progression (Dvinge H. et al., Nat Rev Cancer (2016) (7):413-30). Exemplary cell surface antigens that can be used to in the treatment of Breast Cancer are listed in TABLE 1, TABLE 2, and TABLE 3.

In some embodiments, breast cancer size is diminished after administration of a cancer treatment described herein compared to that in the absence of the administration of the treatment. In some embodiments the treatment comprises a vaccine comprising one or more alternative splicing derived cell surface antigens, TCR engineered T cells specific for an alternative splicing derived neoantigen or cell surface antigen, antibodies, ADCs, Bi and Trispecific antibodies and cell engagers specific for an alternative splicing derived neoantigen, or CAR-T cells specific for an alternative splicing derived cell surface antigen.

Contemplated patients may carry mutations in a splicing factor such as U2AF35, CRSR2, SRSF2, and SF3B1 leading to alternative splicing derived cell surface antigens for example as listed in TABLE 1. Additionally or alternatively, the above described methods and systems may be used to ascertain the presence of a cell surface antigen, as listed for example in TABLE 1. Suitable pharmaceutical compositions can be chosen according to the presence or absence of cell surface antigens. For example, if the cancer cells in a patient are tested positive for a certain cell surface antigen, a suitable pharmaceutical composition can be chosen for treatment.

### Acute Myeloid Leukemia (AML)

In some embodiments, any of the treatments and or methods disclosed herein is for use in treatment of a patient having AML.

Acute myeloid leukemia (AML is a common and fatal form of hematopoietic malignancy characterized by the production of abnormal myeloblasts that infiltrate the bone marrow, blood, and other tissues. AML is the most common hematological malignancy in adults over 65. Survival rates have improved over the last 50 years, however, only 5 to 15% of patients with AML over the age of 60 are cured, with those who cannot tolerate intensive chemotherapy experiencing a dismal median survival of only 5 to 10 months. demonstrating the urgent need for novel therapies. Functional Furthermore, unfavorable treatment outcomes are also associated with certain AML subtypes (Marcucci G. et al., Curr Opin Hematol (2005)12, 68-75, Byrd J. C. et al., Blood (2002) 100, 4325-4336, and Grimwade D. et al., Hematology/oncology clinics of North America (2011) 25, 1135-1161, vii). Recently, IO has revolutionized AML treatments for some patients, as evidenced by the success of allogeneic hematopoietic stem cell transplant (HSCT) and the anti-CD33 antibody drug conjugate gemtuzumab ozogamicin (Liu Y. et al., Blood Rev (2019) 34, 67-83). However, the critical need to develop therapeutics for most AML patients remains. Genomic studies have shown that the pathogenesis of AML is highly heterogeneous with a low TMB. In addition, 40% to 85% of patients with pre-AML dysplasia show mutations in at least one out of 4 splicing factors (U2AF35, CRSR2, SRSF2, and SF3B1).

In some embodiments, AML is diminished after administration of a cancer treatment described herein compared to that in the absence of the administration of the treatment. In some embodiments the treatment comprises a vaccine comprising one or more alternative splicing derived cell surface antigens, TCR engineered T cells specific for an alternative splicing derived neoantigen or cell surface antigen, antibodies, ADCs, bispecific antibody or T cell engager, trispecific antibody or T cell engager specific for an alternative splicing derived cell surface antigen, or CAR-T cells specific for an alternative splicing derived cell surface antigen.

Contemplated patients may carry mutations in a splicing factor such as U2AF35, CRSR2, SRSF2, and SF3B1 leading to alternative splicing derived cell surface antigens for example as listed in TABLE 1. Additionally or alternatively, the above described methods and systems may be used to ascertain the presence of a cell surface antigen, as listed for example in TABLE 1. Suitable pharmaceutical compositions can be chosen according to the presence or absence of cell surface antigens. For example, if the cancer cells in a patient are tested positive for a certain cell surface antigen, then a suitable pharmaceutical composition can be chosen for treatment.

### VI. Tumor-specific Biomarkers

It is contemplated that the cell surface antigens and their corresponding antigen presenting cells (APCs) presenting peptide/MHC complexes and T cells with their respective reactive TCRs can be used in a variety of diagnostic and prognostic approaches. For example, information about a given T cell epitope or group of T cell epitopes and corresponding T cells can be used to determine whether a subject has a certain cancer which may impact patient treatment. In some embodiments, the compositions and methods disclosed herein are used to guide clinical decision making, e.g. treatment selection, identification of prognostic factors, monitoring of treatment response or disease progression, or implementation of preventative measures. For example, the sequences identified as cancer-specific in TABLE 3 can be used to determine if a subject or patient has a certain cancer. In certain embodiments, a cutoff of frequency can be established in which a patient is diagnosed as having a certain cancer if a certain number of cancer-specific T cells are detected from a patient sample.

Using the information provided herein, it is possible to identify a disease-specific cell surface antigen in a cancer patient. As an example, one such method may comprise the steps of (a) obtaining a first RNA-seq data set from a first sample cell and a second RNA-seq data set from a second diseased sample cell; (b) assembling full length mRNA transcript sequences and extracting genomic loci coordinates of the mRNA transcript sequences; (c) clustering of full length mRNA transcript sequences encoded at the same genomic loci and extraction of exon duo or exon trio mRNA sequences; (d) selecting the most representative full length mRNA transcript sequences; (e) identifying stable full length mRNAs transcripts; (f) translating, in silico the stable full length mRNA transcripts into protein isoform sequences; (g) identifying protein isoform sequences that are predicted to be stable; (h) determining B cell antibody accessibility of the protein isoform sequences by using an algorithm to classify the polarity, hydrophobicity, and surface accessibility of peptides derived from the protein isoform sequences; (i) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; (j) generating a first set of antigenic cell surface antigen sequences based on the first RNA-seq data set and a second set of antigenic cell surface antigen sequences based on the second RNA-seq data set ranked by B cell antibody accessibility and T cell antigenicity; and (k) determining unique antigenic cell surface antigen sequences by comparing the first set of antigenic cell surface antigen sequences and the second set of antigenic cell surface antigen sequences and selecting one or more unique cell surface antigen sequences in the second set, thereby identifying one or more cell surface antigens that are disease specific.

In certain embodiments, one such method may comprise the steps of (a) obtaining a first RNA-seq data set from a first sample cell and a second RNA-seq data set from a second diseased sample cell; (b) assembling full length mRNA transcript sequences and extracting genomic loci coordinates of the mRNA transcript sequences; (c) clustering of full length mRNA transcript sequences encoded at the same genomic loci and extraction of exon duo or exon trio mRNA sequences; (d) selecting the most representative full length mRNA transcript sequences; (e) identifying stable full length mRNAs transcripts; (f) translating, in silico the stable full length mRNA transcripts into protein isoform sequences; (g) identifying protein isoform sequences that are predicted to be stable; (h) determining membrane topologies for each protein isoform; (i) filtering for membrane bound protein isoform sequences; (j) determining B cell antibody accessibility of the protein isoform sequences by using an algorithm to classify the polarity, hydrophobicity, and surface accessibility of peptides derived from the protein isoform sequences; (k) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic; (1) generating a first set of antigenic cell surface antigen sequences based on the first RNA-seq data set and a second set of antigenic cell surface antigen sequences based on the second RNA-seq data set ranked by B cell antibody accessibility and T cell antigenicity; and (m) determining unique antigenic cell surface antigen sequences by comparing the first set of antigenic cell surface antigen sequences and the second set of antigenic cell surface antigen sequences and selecting cell surface antigen sequences present in the second set and not the first set; selecting one or more unique cell surface antigen sequences in the second set, thereby identifying one or more cell surface antigens that are disease specific.

The method can further comprise selecting a treatment regimen for the cancer patient based on identified cell surface antigen(s) in the cancer patient. It is contemplated that such a method can be conducted on a plurality of cancer patients, and the resulting information can be used to identify a patient subpopulation having cell surface antigen(s) of interest.

### VII. Kits

In some embodiments, any of the vectors disclosed herein is assembled into a pharmaceutical or diagnostic or research kit to facilitate their use in therapeutic, diagnostic or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1: Prediction of Viable Transcripts and Proteins Produced by Alternative Splicing

This example describes a computer implemented method to predict the likelihood of cellular alternative splicing to produce stable mRNA transcripts resulting in stable protein or peptide expression as potential targets for immunotherapeutics.

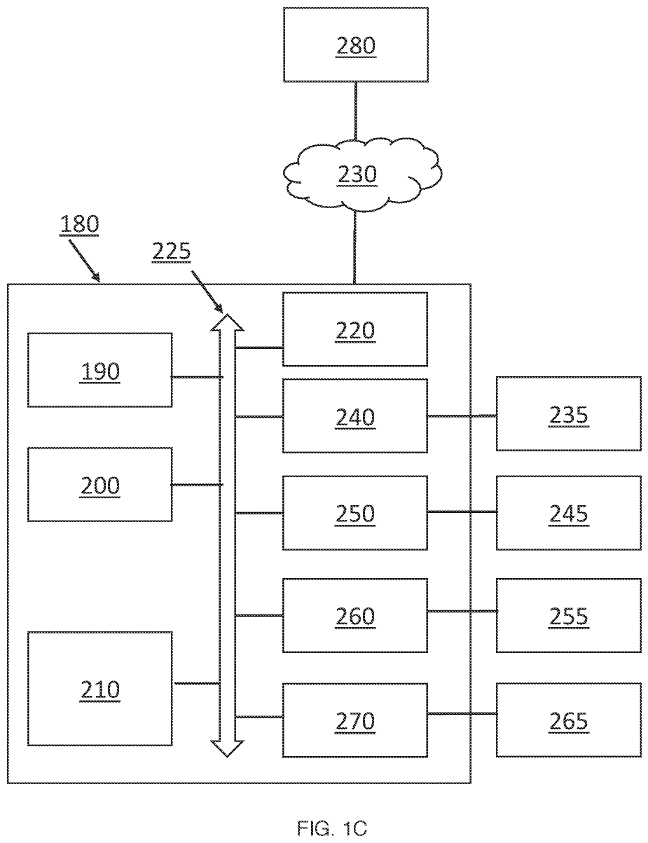

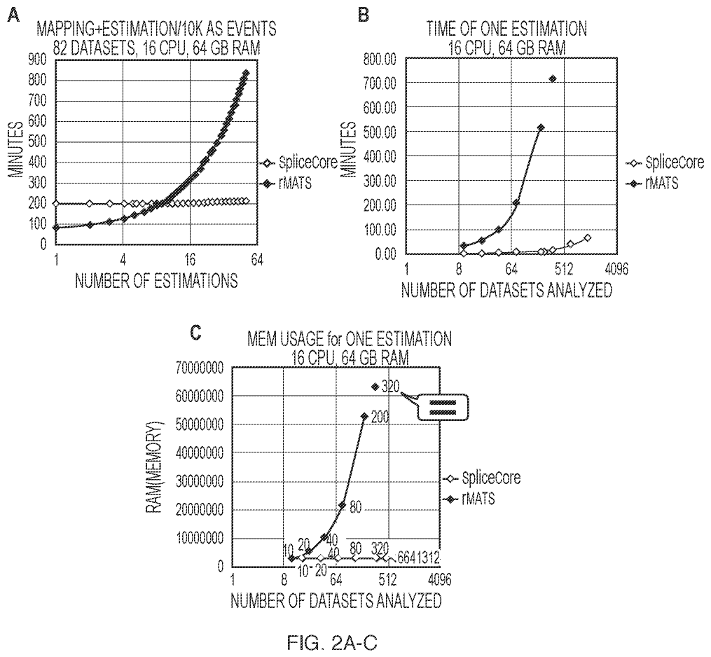

With the recent success of FDA-approved splicing modulators like Nusinersen, splicing research has become of major interest to pharmaceutical companies. Over the last 10 years, Artificial Intelligence (AI) and Machine Learning (ML) have become new tools used by biologists to analyze large and complex datasets such as RNA-seq. For example, high-throughput RNA sequencing can be combined with AI/ML technologies to identify and characterize splicing defects that correlate with disease. SpliceCore (described in PCT/US2019/033574) is an exemplary and innovative cloud-based software platform using biomedical big data for Alternative Splicing (AS) analysis. The SpliceCore platform combines algorithms and databases developed and experimentally validated. SpliceTrap′, for the detection of quantification of alternative splicing using RNA-seq data; SpliceDuo™, for the quantification of significant splicing variation across biological samples; and SpliceImpact™ the detection of AS events that affect protein structure/function and/or RNA stability through NMD. SpliceCore is described in detail in PCT/US2019/033574 and is incorporated by reference herein in its entirety. SpliceCore is a fast, robust and scalable platform to detect alternative splicing events (FIGS. 2A, B, and C).

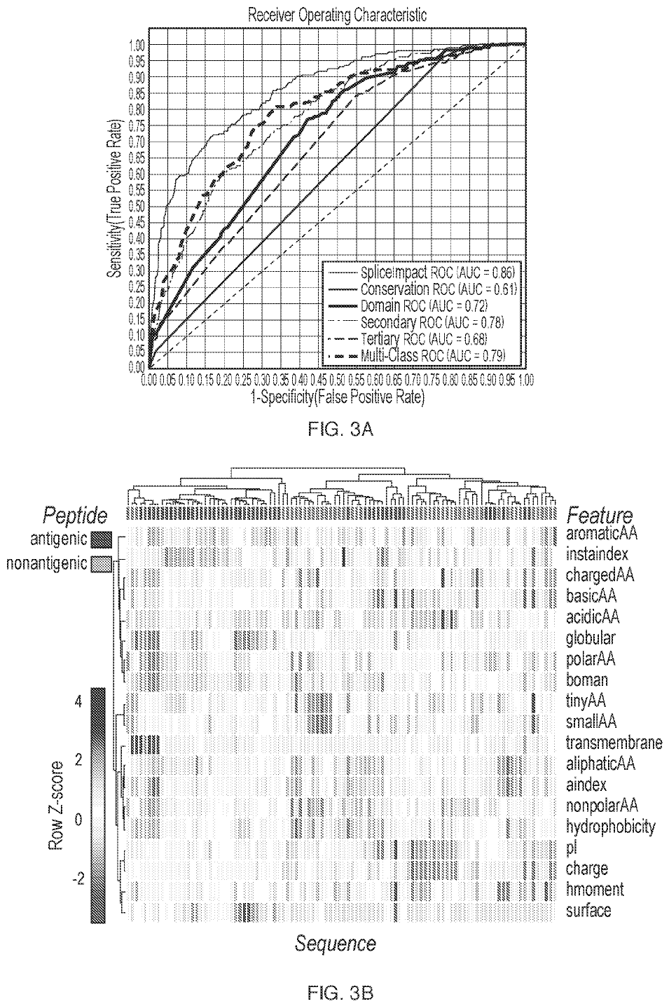

Briefly, SpliceCore, combines transcriptomic and machine learning (ML) analysis to find biologically relevant alternative splicing changes in large amounts of RNA-seq data and to develop therapies targeting splicing regulation defects. In SpliceCore, RNA-seq data is mapped to a proprietary reference database (TXdb), which incorporates at least 7 million splicing events derived from the analysis of public RNA-seq datasets, for example including >10.000 from TCGA with ˜1.500 BRCA breast cancer tissues, and from the Genotype-tissue expression repository (GTEx) with 3.000 normal breast tissues. Splicing events are defined as any combination of 2 or 3 exons in the transcriptome (i.e., exon trios, described in Wu J. et al., Bioinformatics. (2011) (21):3010-6). Every exon trio is represented by two “inclusion” splice junctions and one “skipping” splice junction. TXdb creates a search space for novel junction discovery useful to differentiate self from non-self splice junctions. To prioritize biologically relevant alternative splicing, SpliceCore implements a ML module (SpliceImpact™) that determines whether splicing events impair protein translation through nonsense mediated mRNA decay (NMD), produces unstable truncated peptides, or conversely result in stable proteins that accumulate in significant amounts as shown in FIG. 3A. A pre-requisite for predicting neoantigens and their antigenicity is to prioritize transcripts that are likely to generate polypeptides. SpliceImpact™ is a Machine Learning classifier that enables the effective identification of alternative splicing events likely to disrupt protein viability through open reading frame truncation or nonsense-mediated mRNA decay (NMD). SpliceImpact™ was trained using a gradient boosting method on over 45,000 splicing events from TXdb, a reference database. For training purposes, events were labeled as “stable” or “unstable”. 1,027 AS events encoding minor splicing isoforms were labeled “stable.” Since most coding genes tend to express a single primary protein isoform (see e.g., Ezkurdia I. et al., Most highly expressed protein-coding genes have a single dominant isoform. JProteome Res (2015)14, 1880-1887) the less widely expressed isoform can effectively model sporadic, yet viable AS events. On the other hand, “unstable” AS events were drawn from a pool of 32,692 constitutive exon trios for which there was no evidence of exon skipping in the Intropolis database (described in Nellore A. et al., Human splicing diversity and the extent of unannotated splice junctions across human RNA-seq samples on the Sequence Read Archive. Genome Biol (2016)17, 266). Thus, theoretical skipping of those exons was labelled “unstable” given that those events would be potentially damaging to mRNA stability and its function. In addition, to further account for “unstable” mRNA degradation targets, 15,542 Appris annotations labelled as “NMD” (see in Rodriguez J. M. et al., APPRIS 2017: principal isoforms for multiple gene sets. Nucleic Acids Res (2018).46, D213-D217) were added to the training dataset. SpliceImpact™ was validated on a set of 28 known cancer driving AS events extracted from Urbanski et al., Alternative-splicing defects in cancer: Splicing regulators and their downstream targets, guiding the way to novel cancer therapeutics. Wiley Interdiscip Rev RNA(2018) 9, e1476. Of the 28 AS events, 26 showed an impact probability of >0.5 and 19 of them were >0.75. In addition, 17 of these AS events scored higher than the median of other non-pathological AS events encoded by the same genes.

TABLE 4 Exemplary types of cell surface antigens resulting from Alternative Splicing events and example genes identified.

TABLE 4

| Neoantigen Type | Example gene |
|---|---|
| Intron Retention (IR) | SIRT7, IDS, ENO3, STRN4 |
| Frameshift | SPDYE2B, FLII actin remodeling protein, |
| | MYRFL, HDAC5, DOCK7, SELENOH |
| Translated lncRNAs | RPS6KB2, MCF2L, ATXN3, OFCC1, |
| | ANKRD20A5P, PKP1, BICDL1, LOC101927503 |
| Novel splicing | RGS11, CSF2RB, XRCC5, ABCB1, ZKSCAN7 |
| junction | |
| Novel Exon | ANO6, NUCB2, SERF1B |
| Chimeric | LONP2/SIAH1, CTRBI/-, BMP4/MIR5580, |
| | P2RY10/-, MCF2L, ANO6 |

### Example 2: Prediction of Antigenic and Cell Surface Exposed Cell Surface Antigens

This example describes a computer implemented method termed SpliceIO to identify alternative splicing derived antigenic cell surface antigens and neoantigens. SpliceIO is a predictive ensemble that utilizes exon duos and trios comprising alternative splicing junctions identified by methods such as described in EXAMPLE 1 to predict cell surface antigen antigenicity and membrane topology.

SpliceIO comprises two main ML modules: an “immunoncology” (IO) module to predict antigenicity and a “membrane bound” (MB) module, to predict protein topology and membrane localization.

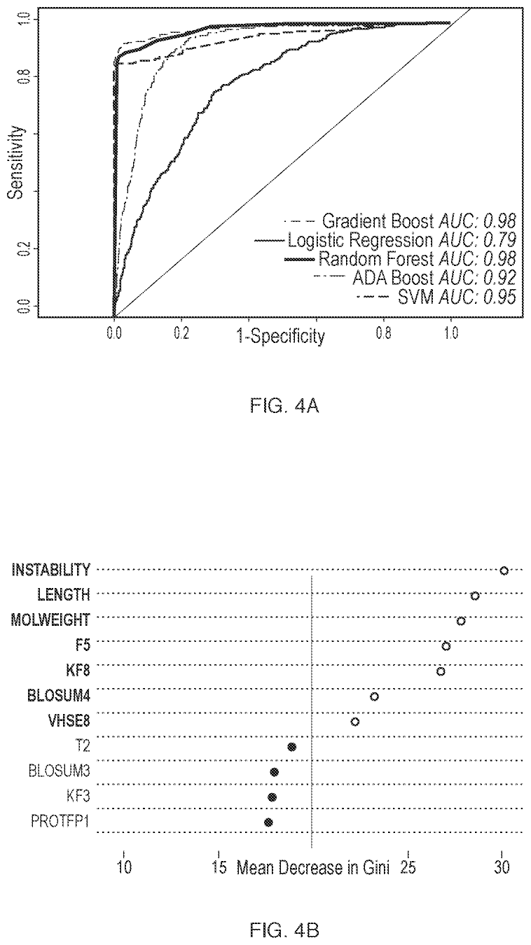

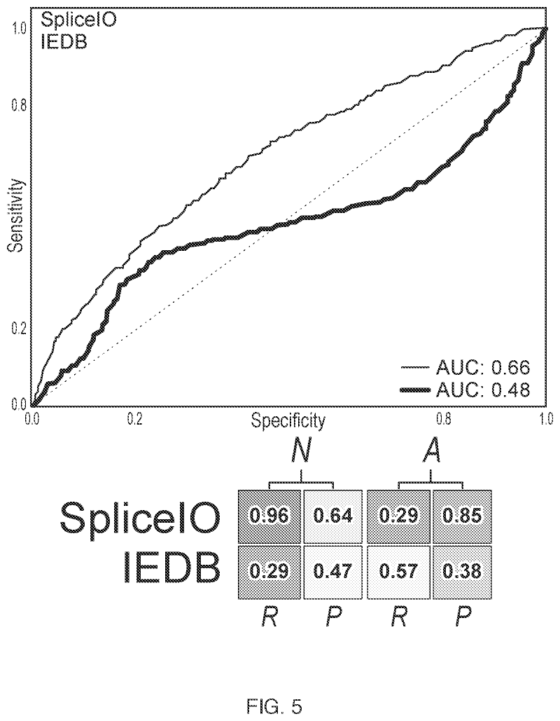

To train and test the IO module, 6751 viral peptide sequences, comprising 1040 antigens and 4387 non-antigens; and 1324 bacterial peptide sequences, comprising 576 antigens and 748 non-antigens were compiled. Antigenic potential of all viral and bacterial peptide sequences had been previously assessed in vitro by cytokine secretion and cytotoxicity, or in vivo by protection from infection (see e.g., Vita R. et al., The Immune Epitope Database (IEDB): 2018 update. Nucleic Acids Res 47, (2019) D339-D343). Peptides that elicited an immune response were classified as “antigens”. A comprehensive set of supervised classifiers for model training, including support vector machine (SVM) and ensemble methods (random forest, gradient boost, AdaBoost) was considered. A grid search with 5-fold cross validation to select and subsequently tune the classifier with optimal performance, as determined by AUC score was performed. Exemplary performance of models is shown in FIG. 3B and FIGS. 4A and 4B. An unsupervised feature weighting by hierarchical clustering performed on known antigenic and non-antigenic peptide sequences from the Immune Epitope Database (IEDB) is shown in FIG. 5. Performance assessment using linear, SVM or ensemble-based models revealed robust predictive capacity across all (FIG. 6A). 77 sequence-based features were considered, comprising biochemical, topological, and conformational peptide descriptors. To reduce computational complexity, feature selection was performed by eliminating highly correlated parameters (Spearman correlation, r>0.7), which resulted in a reduced set of 37 features. Then, a min-max scaler to process each peptide's feature sets for subsequent classifier training, which predominantly relied on biochemical and biophysical properties (FIG. 6B) was used. A separate model trained with viral peptides only and independently tested with bacterial peptides only, corroborated SpliceIO's ability to predict antigenicity, regardless of possible species biases. While the AUC of this particular model was modest (AUC=0.66), it has a precision of 0.85 in predicting antigenic peptides and recall of 0.96 in predicting non-antigens. The widely used IEDB immunogenicity algorithm (see e.g., Vita R. et al., The Immune Epitope Database (IEDB): 2018 update. Nucleic Acids Res 47, (2019) D339-D343), shows random predictive power, suggesting that MHC-binding alone is insufficient to predict antigenicity (FIGS. 6A and 6B).

To train and test the MB module 2,650 protein sequences which were labelled with two characteristics were used. The first were labelled either “membrane bound” or “intracellular”, and the second label was either “with” or “without” signal peptides (61) (FIG. 6A). In performance assessments, SpliceIO's M13 module has equivalent and/or better sensitivity and specificity when compared to Signal P5.0, (FIG. 6B). The MB module accurately predicts protein topology and localization (AUC >0.80, FIGS. 6A and 6B).

Thus, SpliceIO integrates a number of Machine Learning algorithms together to predict for example tumor specific cell surface antigens and neoantigens. These results support the utility of SpliceIO as a robust predictive module for both topology and antigenicity using only peptide sequence-derived features.

To predict cell surface antigens from patient derived RNA-seq data, SpliceIO can use the exon trios identified by SpliceCore in EXAMPLE 1. SpliceIO repurposes the SpliceCore platform's exon duo or exon trio (or exon-centric) approach to analyzing AS events for novel splicing junctions. The resulting novel junctions can be further classified as cell surface antigens using a combination of SpliceCore and SpliceIOs IO module antigenicity from bacterial and viral sequences (see also Schumacher T. N., et al. Neoantigens in cancer immunotherapy. Science. (2015) 348(6230):69-74 and Lu Y-C. et al., Cancer immunotherapy targeting neoantigens. Seminars in Immunology. (2016) 28(1):22-7.), and/or SpliceIOs MB module or an open source tool such as Phobius to predict cell surface antigen membrane topology.

### Example 3: Determination of Tumor Specific Alternative Splicing Events

This example describes the determination of tumor specific alternative splicing events and the identification of novel immunotherapeutic targets. Briefly, TCGA breast cancer RNA-seq data (gdc.cancer.gov/projects/TCGA-BRCA) from 148 patients with 114 HLA alleles was analyzed using SpliceCore and SpliceIO as described in EXAMPLES 1 and 2. The resulting data was compared with the point mutations reported in the data in the Cancer Immunome Atlas (TCIA) (tcia.at/). Almost 3 Million neoantigens resulting from alternative splicing events and filtered for antigenicity were identified using the exon based approach of SpliceCore and SpliceIO, whereas TCIA had annotated about 16000 neoantigens for the same dataset using exome based approaches. Comparing the neoantigens derived from alternative splicing events to the genomic mutation data from the same patients in TCIA, SpliceIO identified 49 common neoantigen candidates (recurrent ninemer-coding neo-junctions) that appeared in 94.5% of the TCGA breast cancer patients, and 1143 neoantigen candidates that appeared in over 100 patients, whereas TCIA reported none. In more than >10 BRCA patients, about 155,000 neoantigens were identified by SpliceIO, whereas only 2 neoantigens were found in >10 BRCA patients using genomic point mutations reported in the Cancer Immunome Atlas (TCIA). The data is summarized in TABLE 5.

TABLE 5

| Patients | SpliceIO | Exome-based (TCIA) |
|---|---|---|
| >1 | 2,844,608 | 15,638 |
| >10 | 155,521 | 2 |
| >50 | 11,662 | 0 |
| >100 | 1,143 | 0 |
| >140 | 49 | 0 |

The cell surface antigens identified by SpliceIO were then compared with pre-processed proteomic tumor profiling data deposited in the Clinical Proteomic Tumor Analysis Consortium (CPTAC), which contains peptides sampled from multiple cancer types (described in Edwards N. J. et al., The CPTAC Data Portal: A Resource for Cancer Proteomics Research. J Proteome Res (2015) 14, 2707-2713). Of the more than 2.8 million neoantigen candidates identified by SpliceIO, 32 were validated using the CPTAC mass spectrometry data (MS/MS). Exemplary cell surface antigen sequences identified by SpliceIO and validated by MS/MS data are shown in TABLE 6.

TABLE 6 exemplary cell surface antigens, parental proteins, and genome location.

TABLE 6

| PEP | Chromosome | SEQ | | | |
|---|---|---|---|---|---|
| ID NO: | location (hg38) | ID NO: | Gene | Chromosome | Strand |
| PEP1 | 113008926- | 1 | N/A | chr13 | + |
| | 113008989.2 | | | | |
| PEP2 | 81917967- | 2 | SIRT7 | chr17 | − |
| | 81918038.1 | | | | |
| PEP3 | 271201- | 3 | RGS11 | chr16 | − |
| | 271315.1 | | | | |
| PEP4 | 149503311- | 4 | IDS | chrX | − |
| | 149504156.4 | | | | |
| PEP5 | 102653206- | 5 | N/A | chr7 | + |
| | 102653402.1 | | | | |
| PEP6 | 36929638- | 6 | N/A | chr22 | + |
| | 36929807.20 | | | | |
| PEP7-1 | 14232524- | 7 | N/A | chr18 | + |
| | 14232629.1 | | | | |
| PEP8-1 | 67434057- | 8 | RPS6KB2 | chr11 | + |
| | 67434170.2 | | | | |
| PEP9 | 216122061- | 9 | XRCC5 | chr2 | + |
| | 216122253.1 | | | | |
| PEP10 | 201324940- | 10 | N/A | chr1 | + |
| | 201325127.25 | | | | |
| PEP11-1 | 87539267- | 11 | N/A | chr7 | − |
| | 87539345.2 | | | | |
| PEP12 | 18254768- | 12 | FLII | chr17 | − |
| | 18254854.1 | | | | |
| PEP13 | 120057016- | 13 | BICDL1, | chr12 | + |
| | 120057187.1 | | CCDC64 | | |
| PEP14 | 92093251- | 14 | ATXN3 | chr14 | − |
| | 92093318.8 | | | | |
| PEP15-1 | 46727546- | 15 | STRN4 | chr19 | − |
| | 46728007.6 | | | | |
| PEP16 | 17330901- | 16 | N/A | chr11 | + |
| | 17330983.17 | | | | |
| PEP17-1 | 9697633- | 17 | LOC112267894, | chr6 | − |
| | 9697797.1 | | OFCC1 | | |
| PEP18-1 | 44078794- | 18 | N/A | chr17 | − |
| | 44078873.1 | | | | |
| PEP19-1 | 48356664- | 19 | LONP2 | chr16 | + |
| | 48356666.1 | | | | |
| PEP20 | 70025633- | 20 | N/A | chr5 | + |
| | 70025657.18 | | | | |
| PEP21 | 69879095- | 21 | MYRFL | chr12 | + |
| | 69879156.1 | | | | |
| PEP22 | 4953050- | 22 | ENO3 | chr17 | + |
| | 4953271.2 | | | | |
| PEP23 | 44568306- | 23 | N/A | chr3 | + |
| | 44568433.116 | | | | |
| PEP24 | 45357289- | 24 | N/A | chr12 | + |
| | 45357424.2 | | | | |
| PEP25 | 62552731- | 25 | DOCK7 | chr1 | − |
| | 62552901.1 | | | | |
| PEP26 | 57741818- | 26 | C11orf31 | chr11 | + |
| | 57741954.1 | | | | |
| PEP27 | 1054238- | 27 | LOC101927503, | chr11 | + |
| | 1054392.1 | | RP13-870H17.3 | | |

TABLE 7 shows exemplary cell surface antigens and associated AS events comprising Retained Introns, Novel Exons, Skipped Exons, Frameshifts, Novel splicing junctions, Noncoding regions, or Fusions.

TABLE 7

| PEP ID | Retained | Novel | Skipped | Frame- | Neo- | Non- | |
|---|---|---|---|---|---|---|---|
| NO: | Intron | Exon | exon | shift | junction | coding | Fusion |
| PEP1 | 1 | 1 | 0 | 0 | 0 | 0 | 0 |
| PEP2 | 1 | 0 | 0 | 0 | 0 | 1 | 0 |
| PEP3 | 0 | 0 | 1 | 0 | 1 | 0 | 0 |
| PEP4 | 1 | 1 | 0 | 0 | 0 | 0 | 0 |
| PEP5 | 0 | 1 | 0 | 1 | 0 | 0 | 0 |
| PEP6 | 0 | 0 | 1 | 1 | 1 | 0 | 0 |
| PEP7 | 0 | 0 | 0 | 0 | 0 | 1 | 1 |
| PEP8 | 1 | 1 | 0 | 0 | 0 | 1 | 0 |
| PEP9 | 0 | 0 | 1 | 0 | 1 | 0 | 0 |
| PEP10 | 0 | 0 | 1 | 0 | 0 | 1 | 0 |
| PEP11 | 0 | 0 | 1 | 0 | 1 | 0 | 0 |
| PEP12 | 0 | 0 | 1 | 1 | 0 | 0 | 0 |
| PEP13 | 0 | 1 | 0 | 0 | 0 | 1 | 0 |
| PEP14 | 0 | 0 | 1 | 0 | 0 | 1 | 0 |
| PEP15 | 1 | 1 | 0 | 0 | 0 | 0 | 0 |
| PEP16 | 0 | 1 | 0 | 0 | 0 | 0 | 0 |
| PEP17 | 0 | 0 | 0 | 0 | 0 | 1 | 0 |
| PEP18 | 0 | 0 | 1 | 1 | 0 | 0 | 0 |
| PEP19 | 0 | 0 | 0 | 0 | 1 | 0 | 1 |
| PEP20 | 0 | 1 | 0 | 0 | 1 | 0 | 0 |
| PEP21 | 0 | 0 | 1 | 1 | 0 | 0 | 0 |
| PEP22 | 1 | 1 | 0 | 0 | 0 | 0 | 0 |
| PEP23 | 0 | 0 | 1 | 1 | 1 | 0 | 0 |
| PEP24 | 0 | 1 | 0 | 0 | 0 | 0 | 0 |
| PEP25 | 0 | 0 | 1 | 1 | 0 | 0 | 0 |
| PEP26 | 0 | 0 | 1 | 1 | 0 | 0 | 0 |
| PEP27 | 0 | 0 | 0 | 0 | 0 | 1 | 0 |

TABLE 8 shows exemplary scoring of cell surface antigen sequences with Modules in SpliceIO.

TABLE 8

| PEP | | | | | | | |
|---|---|---|---|---|---|---|---|
| ID | | | | | | | |
| NO: | MHCPAN | SpliceImpact | SpliceImpact.MIN | SpliceImpact.MAX | viral/bacterial | Hydrophobicity | Hydrophobicity(%) |
| PEP1 | . | 0 | 0.407 | 0.407 | 0 | 2 | 42.86 |
| PEP2 | . | 1 | 0.130 | 0.130 | 0 | 2 | 57.14 |
| PEP3 | . | 0 | 0.652 | 0.652 | 1 | 2 | 71.43 |
| PEP4 | . | 1 | 0.266 | 0.266 | 0 | 1 | 28.57 |
| PEP5 | . | 0 | 0.420 | 0.420 | 0 | 1 | 25 |
| PEP6 | . | 1 | 0.120 | 0.120 | 0 | 0 | 14.29 |
| PEP7 | . | 0 | 0.080 | 0.439 | 0 | 2 | 40 |
| PEP8 | . | 1 | 0.143 | 0.299 | 1 | 1 | 28.57 |
| PEP9 | . | 0 | 0.455 | 0.455 | 2 | 2 | 42.86 |
| PEP10 | . | 1 | 0.072 | 0.072 | 2 | 2 | 57.14 |
| PEP11 | . | 0 | 0.395 | 0.679 | 0 | 1 | 28.57 |
| PEP12 | . | 1 | 0.132 | 0.132 | 0 | 2 | 57.14 |
| PEP13 | YES | 0 | 0.561 | 0.561 | 1 | 1 | 28.57 |
| PEP14 | . | 1 | 0.201 | 0.201 | 2 | 1 | 37.5 |
| PEP15 | . | 1 | 0.078 | 0.309 | 1 | 2 | 42.86 |
| PEP16 | . | 1 | 0.180 | 0.180 | 0 | 2 | 50 |
| PEP17 | YES | 1 | 0.135 | 0.245 | 0 | 2 | 71.43 |
| PEP18 | . | 1 | 0.124 | 0.144 | 0 | 1 | 28.57 |
| PEP19 | . | 0 | 0.096 | 0.415 | 0 | 2 | 53.85 |
| PEP20 | . | 0 | 0.191 | 0.819 | 3 | 1 | 28.57 |
| PEP21 | . | 1 | 0.147 | 0.260 | 0 | 2 | 50 |
| PEP22 | . | 1 | 0.049 | 0.270 | 1 | 1 | 28.57 |
| PEP23 | . | 1 | 0.133 | 0.133 | 0 | 0 | 14.29 |
| PEP24 | . | 0 | 0.431 | 0.431 | 0 | 1 | 28.57 |
| PEP25 | . | 1 | 0.160 | 0.160 | 2 | 0 | 14.9 |
| PEP26 | . | 1 | 0.270 | 0.270 | 0 | 1 | 25 |
| PEP27 | . | 1 | 0.334 | 0.334 | 1 | 2 | 42.86 |

The identified cell surface antigens were scored in SpliceIO for antigenicity, membrane topology and IO modalities. TABLE 9 shows the top10 CPTAC-validated SpliceIO hits, along with the corresponding criteria for IO modality assignment. To filter for CPTAC peptides that reliably validate SpliceIO cell surface antigens, peptides were required to match unique AS neoantigens and not any other isoform expressed at the RNA level (based on RNA-seq gene expression analysis). In addition, selected peptides did not match principal isoforms annotated in Appris regardless of RNA expression (51). The overlapping events identified in CPTAC encoded AS isoforms arising from various splicing mechanisms, including multiple targets containing retained intronic sequences that are of particular interest for neoantigen-based anti-tumor therapeutics (65).

TABLE 9 exemplary scoring for the top10 hits identified by SpliceIO. MHC—MHC dependent, MBA (membrane bound antigen).

TABLE 9

| | | Num- | MB | IO | | |
|---|---|---|---|---|---|---|
| PEP | CPTAC | ber | module | module | MHCPan4.0 | |
| ID. | Cancer | of | Membrane | Anti- | MHC | Modality |
| NO: | Type | Patients | bound | genic | bound | decision |
| PEP8 | KIRC, | 2 | No | X | Weak | MHC |
| | LUAD | | | | | |
| PEP13 | LUAD | 1 | No | X | Weak | MHC |
| PEP14 | OV | 4 | No | X | Weak | MHC |
| PEP15 | LUAD | 1 | No | X | Strong | MHC |
| PEP27 | KIRC, | 2 | No | X | Strong | MHC |
| | UCEC | | | | | |
| PEP22 | LUAD | 1 | No | X | Weak | MHC |
| PEP4 | LUAD | 1 | Yes | X | No | MBA |
| PEP11 | KIRC | 1 | Yes | X | No | MBA |
| PEP24 | KIRC | 1 | Yes | X | No | MBA |
| PEP17 | BRCA | 1 | Yes | X | Strong | MBA |

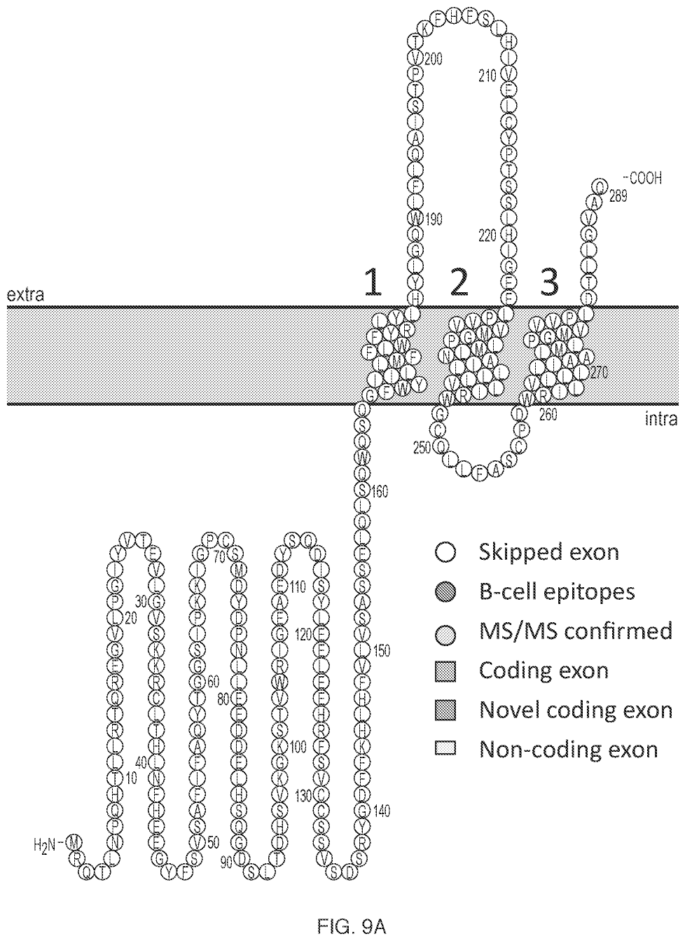

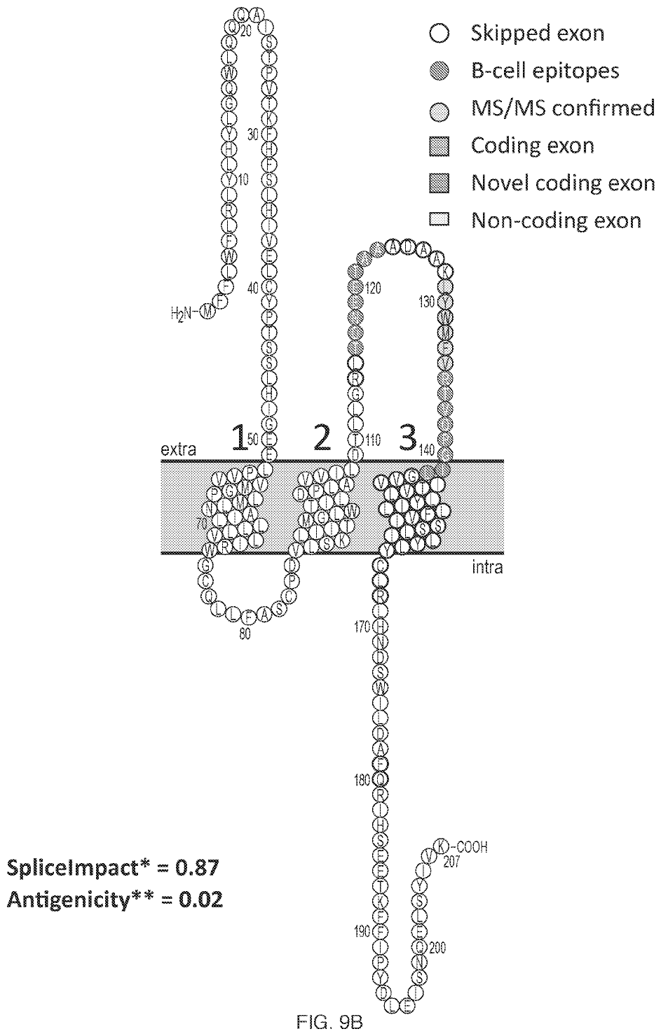

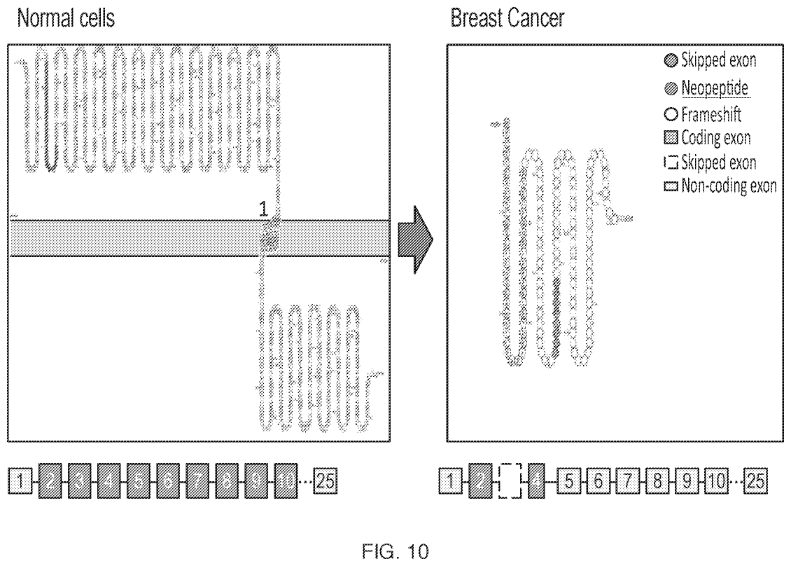

One of the validated hits was SEQ ID NO: 17, an antigenic peptide encoded by the uncharacterized gene LOC112267894. SpliceIO identified this cell surface antigen in one of the 148 patients and predicted this gene to be translated into a transmembrane protein with an antigenic extracellular domain. CPTAC MS data confirmed a stretch of 7 amino acids that could only be explained by the expression of this novel protein. The wild type protein and topology is shown in FIG. 9A, the novel protein in FIG. 9B. Another exemplary protein isoform derived from alternative splicing in breast cancer cells is shown in FIG. 10.

The scoring can further be used to identify if a target is suitable as immunotherapeutic target. A membrane bound cell surface antigen could be targeted for example by antibodies or CAR-T cells. An antigenic MHC bound cell surface antigen could be targeted for example by TCR based therapies such as T cells and TCR engineered T cells, as well as cell surface antigen based vaccines.

These results show that SpliceIO together with SpliceCore can be used to identify and characterize cell surface antigens suitable as novel immunotherapeutic targets.

### Example 4: Use of Patient Organoids for Discovery and Validation of Cell Surface Antigens

This example describes the use of patient-derived organoids for the discovery pf cell surface antigens. Briefly, patient-derived organoids can be used to identify and evaluate BRCA-specific tumor antigens. Tumor organoids are 3D tissue cultures that can be derived from individual patients with a relatively high chance of success (see also Drost J. et al., Translational applications of adult stem cell-derived organoids. Development. (2017) March 15; 144(6):968 and Dutta D. et al., Disease Modeling in Stem Cell-Derived 3D Organoid Systems. Trends Mol Med. (2017); 23(5):393-410). They have become an important and innovative tool for cancer research because of their ability to significantly recapitulate genomic, transcriptomic, proteomic, and histological components of real tumors (see Koren S. et al., Breast Tumor Heterogeneity: Source of Fitness, Hurdle for Therapy. Mol Cell. (2015) 60(4):537-46) and because they can be compared to normal tissues generated from the same patients, thereby minimizing the amount of unspecific variation (described in Aboulkheyr Es H. et al., Personalized Cancer Medicine: An Organoid Approach. Trends Biotechnol (2018) 36(4):358-71 and Jenkins R W. Et al., Ex Vivo Profiling of PD-1 Blockade Using Organotypic Tumor Spheroids. Cancer Discov. (2018) 8(2):196-215). For these reasons, organoids deliver unique value for cancer research, with tremendous potential for the implementation of personalized immuno-oncology. Recently, a similar approach was validated to ex-vivo profile the anti-PD1 therapy using patient tumor spheroids (described in Arun G. et al., Differentiation of mammary tumors and reduction in metastasis upon Malat1 lncRNA loss. Genes Dev. (2016) 30(1):34-51). Most importantly use of patient-derived organoids for multi-omics study is a highly innovative approach that can be further evaluated in the clinical setting for personalized cell surface antigen prediction where the primary tissue material is limited.

### Organoids

Briefly, deidentified patient breast tumor and normal tissues can be processed for establishment of organoids according to the protocol described in Keskin et al. Neoantigen vaccine generates intratumoral T cell responses in phase Ib glioblastoma trial. Nature (2019) 565(7738):234-9. 1/3 of the fresh Tumor/Normal tissue material can be flash frozen for bulk tumor DNA/RNA extraction. Remaining tissues can be processed for organoid generation after collagenase treatment and plating on Matrigel with appropriate growth factors. The organoid cultures can be passaged for a few generations to establish them as a line and the cells can be sampled at different passage points for RNA (2 replicates) and DNA extractions. The lines can be frozen down once they reach the growth phase. Additionally, the cells can be dissociated from the organoids for proteomics analysis. The patient derived organoids can be harvested, and grown. RNA can be extracted and sequenced and cellular proteins can be extracted run tested by tandem mass spectrometry MS/MS for cell surface antigens present in diseased tissue as described in EXAMPLES 1, and 2. The identified cell surface antigens can be scored for antigenicity, membrane topology, and targeting modality as described in EXAMPLE 3. Alternatively, variant cDNA can be overexpressed in patient specific HLA in cell lines and MHC-peptide complex cab be purified from the cell lines to verify the presentation of the identified antigenic peptides translated from mRNA generated from aberrant alternative splicing.

### RNA and DNA Sequencing of Patient-Derived Tumor and Normal Organoids

In order to discover splicing-driven neo-junctions, DNA and RNA-seq of patient tumor-derived organoids from 15-20 different patient samples and corresponding matched normal organoids can be performed. While patient specific cell surface antigens may not be represented by more than 1 patient, and it is a common practice to perform personalized cell surface antigens discovery, 15-20 patient samples should be able to identify any recurring neoantigen events with at least 60% statistical power and FDR <10%. About 500,000 cells can be used for RNA extraction using TRIzol and about 200,000 cells can be utilized to obtain a minimum of 1 ug of DNA per matched pair. Experimental duplicates for stranded paired end RNA-seq libraries from polyadenylated RNAs can be generated using the Illumina TruSeq protocol, and pooled libraries can be sequenced using the Illumina next-seq platform to generate at least 70-100 million reads per sample. This sequencing depth can robustly identify neo-junctions, as SpliceCore analysis often operates with ˜30 million reads and in this case, more than double the sequencing depth to reduce false discovery rate to <=5% can be used. WES using capture probes can be performed on matched tumor/normal pairs using the Illumina TruSeq exome seq protocol. Pooled indexed libraries can be hybridized to capture probes (˜360.000 for ˜20,000 coding genes), barcoded, and subsequently sequenced in the PH 50 Nextseq platform to obtain at least 50 times sequencing depth and coverage.

### Cell Surface Antigen Prioritization and Comparison of RNA-Seq Vs DNA-Seq Methods

Immune stimulation depends on the ability of MHC presented antigens to be recognized by TCRs on cytotoxic T cells. To evaluate the antigenicity of translatable neo-junctions selected with SpliceCore and WES-based methods, two predictive methods for peptide presentation on MHCs Class I and II can be used. The immune epitope dataset (IEDB) is an extensive repository that provides access to known neoantigens as well as predictive algorithms for neoantigen discovery across multiple HLA alleles (Vita R. et al., The immune epitope database (IEDB) 3.0. Nucleic Acids Res. (2015) 43:D405-12). The second tool is NetMHCpan (www.cbs.dtu.dk/services/NetMHCpan/) which predicts binding of peptides to any MHC molecule of known sequence using artificial neural networks (ANNs). Cell surface antigens can be scored for antigenicity as described in EXAMPLE 3.

### Evaluation of the Translation Potential of Identified Peptides

The expression and abundance of cell surface antigen peptides can be evaluated using liquid chromatography coupled tandem mass spectrometry analysis (LC-MS/MS). A database of predicted variant peptide sequences with the theoretical peptide mass fingerprint (PMF) based on the alternative splicing isoforms annotated in TXdb, which also covers and includes all the necessary peptides for mapping WES-based neoantigens can be assembled. The total cell lysates derived from breast tumor and normal organoids can be subjected to LC-MS/MS analysis and cab be used to identify if the targeted peptides are present in tumor cell lysates and quantitatively determine the abundance of the peptides. If the direct lysis method lack sensitivity, samples can be enriched for MHC bound peptides for example by using MHC class I specific antibodies bound to sepharose columns. Approximately 10⁸ cells from organoid culture can be lysed and passed through the column for binding followed by washes and mild acid elution of the MHC-bound peptides. Concentrated peptides in 0.1% formic acid can be subjected to LC-MS/MS. Nano LC can be performed at the flow rate of 200-300 nl/min over 90 min. This can be followed by tandem MS/MS (OrbiTrap) using settings for high targets and long accumulation times for MS2 spectra for an improved spectral quality and spectral yields of up to 30-40% at 100-120 m/s. Data acquisition can be performed as described in (Purcell AW. et al., Mass spectrometry-based identification of MHC-bound peptides for immunopeptidomics. Nat Protoc. (2019) 14(6):1687-707). Finally, the peptides can be searched against the custom PMF database using MAXQuant (www.maxquant.org/) software after application of the 5% FDR (low stringency), 1% FDR (high stringency) and 0.05 delta mass cutoff values.

Nonsense Mediated Decay (NMD) Assays to Evaluate the Proportion of Variant Isoforms Triggering NMD with Peptide Presentation

NMD is one of the key mechanisms of RNA quality control and functions at the level of translation (55). Improperly spliced RNAs vs. RNA with retained introns, undergo nonsense mediated decay after the pioneering round of translation. The peptides generated at the pioneering round of translation undergo proteasomal degradation and may be presented on the MHC (56). For mRNAs expected to undergo NMD based on SpliceImpact predictions, the organoids can be treated with cycloheximide to arrest protein synthesis and accumulation of NMD targets can be evaluated using RT-PCR. mRNAs undergoing NMD can accumulate upon translation inhibition as NMD is coupled to translation. NMD transcripts can be quantitatively validate with/without cycloheximide.

### Analysis of Proteo-Genomic and Proteo-Transcriptomic Data to Experimentally Confirm the Expression of Antigenic Peptides.

Proteomics data can be scored for peptides represented from the WES-based and SpliceCore analysis to rank order candidates based on peptide expression, length (7-11 aa) and sequence similarity to known antigenic sequence using pBLAST (bacterial or viral peptides). Adjusted p value of less than 0.01 and FDR (<1%) can be considered significant hits. Identified peptides can be compared to the CPTAC and IEDB database to identify recurrence of any identified MHC presented peptides.

### Example 5: Identification of Cell Surface Antigens for Vaccine Development

This example describes the identification of cell surface antigen sequences and derived from patient cells or organoids for the use in vaccine development.

Briefly, DNA and RNA sequences can be identified as described in EXAMPLE 4.

In order to develop a vaccine, immunogenic sequences that can be displayed by the MHC and recognized by human T cells can be identified using T cell epitope prediction tools such as mass spectrometry based HLA I and HLA II epitope binding prediction tools (e.g., Immune Epitope Database and Analysis Resource, www.iedb.org). Epitopes such as for HLA-I can be scored for immunogenicity. Top-ranking peptides can be prioritized based on expected population coverage and depending on HLA allele frequencies. Predicted peptides can be tested for T cell responses using PBMCs from human donors and MHC multimers loaded with peptides and then ranked. Further assays of T cell reactivity (e.g., interferon-gamma ELISpots, tetramers), which are stricter measures for T cell immunogenicity to epitopes, can be performed to further identify top immunogenic peptides.

The top peptides can then be further used to develop vaccines, such as mRNA or adenovirus based vaccines.

### Example 6: Identification and Expansion of Cell Surface Antigen-Specific Memory T-Cells from a Patient Sample for T-Cell Therapy

This example describes the selection and expansion of cell surface antigen specific T cells from patient samples. Briefly, T cells can be collected for example by apheresis from a patient. To expand cell surface antigen specific T cells, one or more cell surface antigen peptides that are identified to be presented by the patients cancer cells are identified as described in EXAMPLE 4. The patient derived T cells can be tetramer/multimer sorted ex vivo, activated, and expanded as described in Dudley et al., Clin. Cancer. Res. (2010) 16(24):6122-6131.

The selected and expanded cells can then be further processed and used for T cell based therapies.

### Example 7: Membrane Bound Protein Isoform Specific Antibodies

This example describes the design and identification of antibodies specific to membrane bound protein isoforms derived from alternative splicing. The derived antibodies can for example be used to target cancer cells by engaging cell surface antigens differentially expressed in cancers.

Antibody therapeutics represent the fastest growing class of drugs on the market. Currently 76 antibody-based therapeutics are used in the clinic, with nearly as many in late stages of clinical trials. The most fruitful applications of antibodies lie in the fields of oncology where built-in effector functions help to eliminate tumor cells. A general overview over therapeutic antibodies is in Lu R-M. et al, J Biomed Sci. (2020); 27: 1 and Goulet D. et al., J Pharm Sci. (2020); 109(1): 74-103.

Briefly, mouse or human monoclonal antibodies can be generated for each of the specific epitopes corresponding to the full length protein isoform described in TABLE 10.

TABLE 10

| PEP ID NO: | Protein Name | SEQ ID NO: |
|---|---|---|
| 4 | IDS | 31 |
| 11-1 | N/A | 41 |
| 11-2 | ABCB1 | 42 |
| 11-3 | N/A | 41 |
| 17 | LOC112267894, OFCC1 | 48 |
| 24 | N/A | 58 |

Mouse monoclonal antibodies can be humanized. Rapid amplification of cDNA ends (RACE) can be used to amplify the variable domains of the heavy and light IgG chains, VH and VL can be amplified from the functionally validated murine or human antibodies. Mouse-human chimeric antibodies can be constructed by cloning the VH and VL together with human Ig fragments into plasmid vectors that can be used to overexpress and purify the antibodies in a cell line such as CHO or HEK cells.

Antibodies can be tested for specific binding to the cell surface antigen or cells expressing the cell surface antigen by using methods for example such as ELISA, Biacore™ Octet®, or Isothermal Titration calorimetry (ITC). Selected antibodies can be further tested for biological function in vivo. Additionally or alternatively antibodies can be coupled with to a drug entity forming an antibody drug conjugate (ADC) that combine monoclonal antibodies specific to surface antigens present on particular tumor cells with highly potent anti-cancer agents linked via a chemical linker.

Selected antibodies and ADCs can be manufactured and further administered to the patient having a cancer expressing the cell surface antigen as immune therapy.

### Example 8: Cell Surface Antigen-Specific Chimeric Antigen Receptor T (CAR-T) Cells

This example describes the engineering of CAR-T cells specific for a selected cell surface antigen.

Adoptive cell therapy using naturally occurring endogenous tumor-infiltrating lymphocytes or T cells genetically engineered to express Chimeric Antigen Receptors (CARs) have emerged as promising cancer immunotherapy strategies with remarkable responses in patients with acute lymphoblastic leukemia and other clinical trials (reviewed in Wang X. et al., Molecular Therapy Oncolytics (2016) 3, 16015). Briefly, peripheral blood mononuclear cells are collected from a patient or a healthy donor by a leukapheresis process. T cells are isolated, purified, and activated. The ex vivo expansion of T cells requires sustained and adequate activation. T-cell activation needs a primary specific signal via the T-cell receptor (Signal 1) and costimulatory signals such as CD28, 4-1BB, or OX40 (Signal 2). After the T cells are activated, cells are engineered in order to express a Chimeric Antigen Receptor (CAR) specific for one or more of the identified cell surface antigens. Exemplary membrane bound cell surface antigens as described in EXAMPLE 3 and exemplary antibodies as described in EXAMPLE 7 can be used to design CAR constructs specific for a selected cell surface antigens. The CAR constructs can be cloned into gene expression vectors for use in gamma-retroviral vectors, lentiviral vectors, AAV vectors, or the transposon/transposase system in isolated T cells. CAR constructs can be further expressed as a temporary/transient gene expression from messenger RNA in T cells. These CAR-T cells expressing CARs that specifically target the identified cell surface antigens described in EXAMPLE 3, can be expanded and administered to the patient having a cancer expressing the cell surface antigens as immune therapy.

### Example 9: Cell Surface Antigen-Specific T Cell Receptor (TCR) Cells

This example describes the engineering of T cell receptors and T cells for a T Cell Receptor (TCR) cells specific for a cell surface antigen.

Adoptive T cell therapy (ACT) with T cells expressing native or transgenic αβ-T cell receptors (TCRs) is a promising treatment for cancer, as TCRs cover a wide range of potential target antigens. Transgenic TCR-based ACT allows the genetic redirection of T cell specificity in a highly specific and reproducible manner and has produced promising results in melanoma and several solid tumors.

Briefly, similarly to antibodies, T cell receptors (TCRs) can be engineered for specificity to a selected cell surface antigen. Specificity and affinity of the engineered TCR can be measured in assays, for example tetramer assays, Enzyme Linked Immuno Spot assays (ELISpot), or an Activation Induced Marker (AIM) assay. T cells can be collected from patients, isolated, purified, and activated as described in EXAMPLE 8. The activated T cells can be engineered in order to generate transgenic T cell receptors specific for any of the identified cell surface antigens described in EXAMPLE 3. A transfection vector and/or a CRISPR gene editing system can be designed to generate TCR engineered T cells specific for the selected cell surface antigen.

TCR engineered T cells can be expanded, manufactured, and administered to the patient having a cancer expressing the cell surface antigen as immune therapy.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features herein before set forth.

SEQUENCE LISTING

| SEQ ID | | |
|---|---|---|
| NO: | Sequence | Source |
| 1 | ACIREPR | Homo Sapiens |
| 2 | ARPCPAR | Homo Sapiens |
| 3 | AVAAPTK | Homo Sapiens |
| 4 | AWCSEGR | Homo Sapiens |
| 5 | DENSQLGR | Homo Sapiens |
| 6 | DSWEGGR | Homo Sapiens |
| 7 | ENLTSIVLNSKYIPK | Homo Sapiens |
| 8 | EWGQGPR | Homo Sapiens |
| 9 | FFESLRK | Homo Sapiens |
| 10 | FLSILCS | Homo Sapiens |
| 11 | GGFTFGK | Homo Sapiens |
| 12 | HHPQPAL | Homo Sapiens |
| 13 | LEEESFR | Homo Sapiens |
| 14 | LGKQTAAK | Homo Sapiens |
| 15 | LLCLQGR | Homo Sapiens |
| 16 | LRMEELWR | Homo Sapiens |
| 17 | LYWMEVR | Homo Sapiens |
| 18 | NTGAVCR | Homo Sapiens |
| 19 | QQANMLPPTERVL | Homo Sapiens |
| 20 | RASLCGK | Homo Sapiens |
| 21 | RLSQLPLK | Homo Sapiens |
| 22 | SAQTGLS | Homo Sapiens |
| 23 | SGSEEVR | Homo Sapiens |
| 24 | SPDSTLR | Homo Sapiens |
| 25 | SPGYGSK | Homo Sapiens |
| 26 | SSGLGLRR | Homo Sapiens |
| 27 | VWGAGRR | Homo Sapiens |
| 28 | MCAEDTLQGILTPACIREPRSCGRGSVERERSSGDGPQGLRAGGRGSVE | Homo Sapiens |
| | SGERSSGDDPQRRLAKCGCASPPCPRRKLSHSKGRPEGSAGRLCTDTCP | |
| | PRGSPPAPGPCRLVLRV | |
| 29 | MAAGGLSRSERKAAERVRRLREEQQRERLRQVRRRRSPARPCPARAAAH | Homo Sapiens |
| | RPPARRCRAS | |
| 30 | MERVVVSMQDPDQGVKMRSQRLLVTVIPHAVTGSDVVQWLAQKFCVSEE | Homo Sapiens |
| | EALHLGAVLVQHGYIYPLRDPRSLMLRPDETPYRFQTPYFWTSTLRPAA | |
| | ELDYAIYLAKKNIRKRGTLVDYEKDCYDRLHKKINHAWDLVLMQAREQL | |
| | RAAKQRSKGDRLVIACQEQTYWLVNRPPPGAPDVLEQGPGRGSCAASRV | |
| | LMTKSADFHKREIEYFRKALGRTRVKSSVCLEAVAAPTKLRVERWGFSF | |
| | RELLEDPVGRAHFMDFLGKEFSGENLSFWEACEELRYGAQAQVPTLVDA | |
| | VYEQFLAPGAAHWVNIDSRTMEQTLEGLRQPHRYVLDDAQLHIYMLMKK | |
| | DSYPRFLKSDMYKALLAEAGIPLEMKRRVFPFTWRPRHSSPSPALLPTP | |
| | VEPTAACGPGGGDGVA | |
| 31 | MPPPRTGRGLLWLGLVLSSVCVALGSETQANSTTDALNVLLIIVDDLRP | Homo Sapiens |
| | SLGCYGDKLVRSPNIDQLASHSLLFQNAFAQVCLGTSSCGCVLLRALRV | |
| | GGGELQLLSDGTDCAGHLVRGKHSLENGSGENLVFHSWESLTFKGGLLL | |
| | GCKVRPGPDVFMAALASFLPERALAWCSEGRGAAEGHPQVCRLGLR | |
| 32 | MDRTETRFRKRGQITGKITTSRQPHPQNEQSPQRSTSGYPLQEVVDDEM | Homo Sapiens |
| | LGPSGTQRARDQGRTGSSVRRTEREKNGEGKERHMGLSRGENQKDGLEK | |
| | PAVCKSGEDGEWFGVLGRGLRSLGWKRKREWSDESEEEPEKELAPEPEE | |
| | TWVVEMLCGLKMKLKQQRVSSILPEHHKDFNSQLGRRIPQRAPPILFFL | |
| | KRGNFQ | |
| 33 | MVLAQGLLSMALLALCWERSLAGAEETIPLQTLRCYNDYTSHITCRWAD | Homo Sapiens |
| | TQDAQRLVNVTLIRRVNEDLLEPVSCDLSDDMPWSACPHPRCVPRRCVI | |
| | PCQSFVVTDVDYFSFQPDRPLGTRLTVTLTQHVQPPEPRDLQISTDQDH | |
| | FLLTWSVALGSPQSHWLSPGDLEFEVVYKRLQDSWEGGRVLPSAEGGAR | |
| | QPPHQAPLPDSRARPRDPRPIHRLCSAKEGRETHKELSEHPDGPSIPQR | |
| | DQGWRQLQPALGNNENAIRTHRPHI | |
| 34 | MENNMVELSKLQEYKLELDERAMQAVEKLEEIHLQKQAQYEKQLEQLNK | Homo Sapiens |
| | DNTASLNMKELTLKDVECKFSKMKTTYEEVTTKLEEYKEAFAAALNANN | |
| | SMSKKLTKSNKKIAMISTKLLMEKEWVKYFLSTLPTRRGQESPCVENLT | |
| | SIVLNSKYIPKMTVRIPTSNPQTSNNCQNYLTEMELDCVEQIIRETKRS | |
| | MLPKFIN | |
| 35 | MRVGGVRPPRATDMKKDVQILVVGEPRVGKTSLIMSLVSEEFPEEVPPR | Homo Sapiens |
| | AEEITIPADVTPERVPTHIVDYSEAEQSNEQLHQEISQANVVCIVYAVN | |
| | NKHSIDKKQAQYEKQLEQLNKDNTASLNMKELTLKDVECKFSKMKTTYE | |
| | EVTTKLEEYKEAFAAALNANNSMSKKLTKSNKKIAMISTKLLMEKEWVK | |
| | YFLSTLPTRRGQESPCVENLTSIVLNSKYIPKMTVRIPTSNPQTSNNCQ | |
| | NYLTEVSY | |
| 36 | MKVLRKAKIVRNAKDTAHTRAERNILESVKHPFIVELAYAFQTGGKLYL | Homo Sapiens |
| | ILECLSGGELFTHLEREGIFLEDTACFYLAEITLALGHLHSQGIIYRDL | |
| | KPENIMLSSQGHIKLTDFGLCKESTHEGAVTHTFCGTIEYMAPEILVRS | |
| | GHNRAVDWWSLGALMYDMLTGSPPFTAENRKKTMDKIIRGKLALPPYLT | |
| | PDARDLVKKFLKRNPSQRIGGGPGDAADVQVGLGPPPGVGLSLQGCREW | |
| | GOGPRAEGVTGGQAG | |
| 37 | MAAVFDLDLETEEGSEGEGEPELSPADACPLAELRAAGLEPVGHYEEVE | Homo Sapiens |
| | LTETSVNVGPERIGPHCFELLRVLGKGGYGKVFQVRKVQGTNLGKIYAM | |
| | KVLRKAKIVRNAKDTAHTRAERNILESVKHPFIVELAYAFQTGGKLYLI | |
| | PENIMLSSQGHIKLTDFGLCKESIHEGAVTHTFCGTIEYMAPEILVRSG | |
| | HNRAVDWWSLGALMYDMLTGSPPFTAENRKKTMDKIIRGKLALPPYLTP | |
| | DARDLVKKFLKRNPSQRIGGGPGDAADVQVGLGPPPGVGLSLQGCREWG | |
| | QGPRAEGVTGGQAG | |
| 38 | MVRSGNKAAVVLCMDVGFTMSNSIPGIESPFEQAKKVITMFVQRQVFAE | Homo Sapiens |
| | NKDEIALVLFGTDGTDNPLSGGDQYQNITVHRHLMLPDFDLLEDIESKI | |
| | QPGSQQADFLDALIVSMDVIQHETIGKKFEKRHIEIFTDLSSRFSKSQL | |
| | DIIIHSLKKCDISLQFFESLRKLCVFKKIERHSIHWPCRLTIGSNLSIR | |
| | IAAYKSILQERVKKTWTVVDAKTLKKEDIQKETVYCLNDDDETEVLKED | |
| | IIQGFRYGSDIVPFSKVDEEQMKYKSEGKCFSVLGFCKSSQVQRRFFMG | |
| | NQVLKVFAARDDEAAAVALSSLIHALDDLDMVAIVRYAYDKRANPQVGV | |
| | AFPHIKHNYECLVYVQLPFMEDLRQYMFSSLKNSKKYAPTEAQLNAVDA | |
| | LIDSMSLAKKDEKTDTLEDLFPTTKIPNPRFQRLFQCLLHRALHPREPL | |
| | PPIQQHIWNMLNPPAEVTTKSQIPLSKIKTLFPLIEAKKKDQVTAQEIF | |
| | QDNHEDGPTAKKLKTEQGGAHFSVSSLAEGSVTSVGSVNPAENFRVLVK | |
| | QKKASFEEASNQLINHIEQFLDTNETPYFMKSIDCIRAFREEAIKFSEE | |
| | QRENNFLKALQEKVEIKQLNHFWEIVVQDGITLITKEEASGSSVTAEEA | |
| | KKFLAPKDKPSGDTAAVFEEGGDVDDLLDMI | |
| 40 | MNHSPLKTALAYECFQDQDNSTLALPSDQKMKTGTSGRQRVQEQVMMTV | Homo Sapiens |
| | KROKSKSSQSSTLSHSNRGSMYDGLADNYNYGTTSRSSYYSKFQAGNGS | |
| | WGYPTYNGTLKREPDNRRFSSYSQMENWSRHYPRGSCNTTGAGSDICFM | |
| | QKIKASRSEPDLYCDPRGTLRKGTLGSKGQKTTQNRYSFYSTCSGQKAI | |
| | KKCPVRPPSCASKQDPVYIPPISCNKDLSFGHSRASSKICSEDIECSGL | |
| | TIPKAVQYLSSQDEKYQAIGAYYIQHTCFQDESAKQQVYQLGGICKLVD | |
| | LLRSPNQNVQQAAAGALRNLVFRSTTNKLETRRQNGIREAVSLLRRTGN | |
| | AEIQKOLTGLLWNLSSTDELKEELIADALPVLADRVIIPFSGWCDGNSN | |
| | MSREVVDPEVFFNATGCLRNLSSADAGRQTMRNYSGLIDSLMAYVQNCV | |
| | AASRCDDKSVENCMCVLHNLSYRLDAEVPTRYRQLEYNARNAYTEKSST | |
| | GCFSNKSDKMMNNNYDCPLPEEETNPKGSGWLYHSDAIRTYLNLMGKSK | |
| | KDATLEACAGALQNLTASKGLMSSGMSQLIGLKEKGLPQIARLLQSGNS | |
| | DVVRSGASLLSNMSRHPLLHRVMGRYDPAEKPSGLAGWGFLSILCSIWE | |
| | SSQETEKKPKNCG | |
| 41 | MDLEGDRNGGAKKKNFFKLNNKSEKDKKEKKPTVSVFSMFRYSNWLDKL | Homo Sapiens |
| | YMVVGTLAAIIHGAGLPLMMLVFGEMTDIFANAGNLEDLMSNITNRSDI | |
| | NDTGFFMNLEEDMTRYAYYYSGIGAGVLVAAYIQVSFWCLAAGRQIHKI | |
| | RKQFFHAIMRQEIGWFDVHDVGELNTRLTDDVSKINEGIGDKIGMFFQS | |
| | MATFFTGFIVGFTRGWKLTLVILAISPVLGLSAAVWAKILSSFTDKELL | |
| | AYAKAGAVAEEVLAAIRTVIAFGGQKKELERYNKNLEEAKRIGIKKAIT | |
| | ANISIGAAFLLIYASYALAFWYGTTLVLSGEYSIGQVLTVFFSVLIGAF | |
| | SVGQASPSIEAFANARGAAYEIFKIIDNKPSIDSYSKSGHKPDNIKGNL | |
| | EFRNVHFSYPSRKEVKILKGLNLKVQSGQTVALVGNSGCGKSTTVQLMQ | |
| | RLYDPTEGMVSVDGQDIRTINVRFLREIIGVVSQEPVLFATTIAENIRY | |
| | GRENVTMDEIEKAVKEANAYDFIMKLPHKFDTLVGERGAQLSGGQKQRI | |
| | AIARALVRNPKILLLDEATSALDTESEAVVQVALDKARKGRTTIVIAHR | |
| | LSTVRNADVIAGFDDGVIVEKGNHDELMKEKGIYFKLVTMQTAGNEVEL | |
| | ENAADESKSEIDALEMSSNDSRSSLIRKRSTRRSVRGSQAQDRKLSTKE | |
| | ALDESIPPVSFWRIMKLNLTEWPYFVVGVFCAIINGGLQPAFAIIFSKI | |
| | IGGFTFGKAGEILTKRLRYMVFRSMLRQDVSWFDDPKNTTGALTTRLAN | |
| | DAAQVKGAIGSRLAVITQNIANLGTGIIISFIYGWQLTLLLLAIVPIIA | |
| | IAGVVEMKMLSGQALKDKKELEGSGKIATEAIENFRTVVSLTQEQKFEH | |
| | MYAQSLQVPYRNSLRKAHIFGITFSFTQAMMYFSYAGCFRFGAYLVAHK | |
| | LMSFEDVLLVFSAVVFGAMAVGQVSSFAPDYAKAKISAAHIIMIIEKTP | |
| | LIDSYSTEGLMPNTLEGNVTFGEVVFNYPTRPDIPVLQGLSLEVKKGQT | |
| | LALVGSSGCGKSTVVQLLERFYDPLAGKVLLDGKEIKRLNVQWLRAHLG | |
| | IVSQEPILFDCSIAENIAYGDNSRVVSQEEIVRAAKEANIHAFIESLPN | |
| | KYSTKVGDKGTQLSGGQKQRIAIARALVRQPHILLLDEATSALDTESEK | |
| | VVQEALDKAREGRTCIVIAHRLSTIQNADLIVVFQNGRVKEHGTHQQLL | |
| | AQKGIYFSMVSVQAGTKRQ | |
| 42 | MDLEGDRNGGAKKKNFFKLNNKSEKDKKEKKPTVSVFSMFRYSNWLDKL | Homo Sapiens |
| | YMVVGTLAAIIHGAGLPLMMLVFGEMTDIFANAGNLEDLMSNITNRSDI | |
| | NDTGFFMNLEEDMTRYAYYYSGIGAGVLVAAYIQVSFWCLAAGRQIHKI | |
| | RKQFFHAIMRQEIGWFDVHDVGELNTRLTDDVSKINEGIGDKIGMFFQS | |
| | MATFFTGFIVGFTRGWKLTLVILAISPVLGLSAAVWAKILSSFTDKELL | |
| | AYAKAGAVAEEVLAAIRTVIAFGGQKKELERYNKNLEEAKRIGIKKAIT | |
| | ANISIGAAFLLIYASYALAFWYGTTLVLSGEYSIGQVLTVFFSVLIGAF | |
| | SVGQASPSIEAFANARGAAYEIFKIIDNKPSIDSYSKSGHKPDNIKGNL | |
| | EFRNVHFSYPSRKEVKILKGLNLKVQSGQTVALVGNSGCGKSTTVQLMQ | |
| | RLYDPTEGMVSVDGQDIRTINVRFLREIIGVVSQEPVLFATTIAENIRY | |
| | GRENVTMDEIEKAVKEANAYDFIMKLPHKFDTLVGERGAQLSGGQKQRI | |
| | AIARALVRNPKILLLDEATSALDTESEAVVQVALDKARKGRTTIVIAHR | |
| | LSTVRNADVIAGFDDGVIVEKGNHDELMKEKGIYFKLVTMQTAGNEVEL | |
| | ENAADESKSEIDALEMSSNDSRSSLIRKRSTRRSVRGSQAQDRKLSTKE | |
| | ALDESIPPVSFWRIMKLNLTEWPYFVVGVFCAIINGGLQPAFAIIFSKI | |
| | IGGFTFGKAGEILTKRLRYMVFRSMLRQDVSWFDDPKNTTGALTTRLAN | |
| | DAAQVKGAIGSRLAVITQNIANLGTGIIISFIYGWQLTLLLLAIVPIIA | |
| | IAGVVEMKMLSGQALKDKKELEGSGKIATEAIENFRTVVSLTQEQKFEH | |
| | MYAQSLQVPYRNSLRKAHIFGITFSFTQAMMYFSYAGCFRFGAYLVAHK | |
| | LMSFEDVLLVFSAVVFGAMAVGQVSSFAPDYAKAKISAAHIIMIIEKTP | |
| | LIDSYSTEGLMPNTLEGNVTFGEVVENYPTRPDIPVLQGLSLEVKKGQT | |
| | LALVGSSGCGKSTVVQLLERFYDPLAGKVLLDGKEIKRLNVQWLRAHLG | |
| | IVSQEPILFDCSIAENIAYGDNSRVVSQEEIVRAAKEANIHAFIESLPN | |
| | KYSTKVGDKGTQLSGGQKQRIAIARALVRQPHILLLDEATSALDTESEK | |
| | VVQEALDKAREGRTCIVIAHRLSTIQNADLIVVFQNGRVKEHGTHQQLL | |
| | AQKGIYFSMVSVQAGTKRQ | |
| 43 | MEATGVLPFVRGVDLSGNDFKGGYFPENVKAMTSLRWLKLNRTGLCYLP | Homo Sapiens |
| | EELAALQKLEHLSVSHNNLTTLHGELSSLPSLRAIVARANSLKNSGVPD | |
| | DIFKLDDLSVLHRHHPQPALHQPH | |
| 44 | MSAFCLGLVGRASAPAEPDSACCMELPAAAGDAVRSPAAAAALIFPGGS | Homo Sapiens |
| | GELELALEEELALLAAGERPSDPGEHPQAEPGSLAEGAGPQPPPSQDPE | |
| | LLSVIRQKEKDLVLAARLGKALLERNQDMSRQYEQMHKELTDKLEHLEQ | |
| | EKHELRRRFENREGEWEGRVSELESDVKQLQDELERQQIHLREADREKS | |
| | RAVQELSEQNQRLLDQLSRVGMVTAMDALEEESFRLSSSTSDAEFDAVV | |
| | VYLEDIIMDDRFPIITEKLHGQVLLGLASEVERQLSMQVHALREDFREK | |
| | NSSTNQHIIRLESLQAEIKMLSDRKRELEHRLSATLEENDLLQGTVEEL | |
| | QDRVLILERQGHDKDLQLHQSQLELQEVRLSCRQLQVKVEELTEERSLQ | |
| | SSAATSTSLLSEIEQSMEAEELEQEREQLRLOLWEAYCQVRYLCSHLRG | |
| | NDSADSAVSTDSSMDESSETSSAKDVPAGSLRTALNELKRLIQSIVDGM | |
| | EPTVTLLSVEMTALKEERDRLRVTSEDKEPKEQLQKAIRDRDEAIAKKN | |
| | AVELELAKCRMDMMSLNSQLLDAIQQKLNLSQQLEAWQDDMHRVIDRQL | |
| | MDTHLKERSQPAAALCRGHSAGRGDEPSIAEGKRLFSFFRKI | |
| 45 | EGGVTSEDYRTFLQQPSGNMDDSGFFSIQVISNALKVWGLELILFNSPE | Homo Sapiens |
| | YQRLRIDPINERSFICNYKEHWFTVRKLGKQTAAKAATAAAAAAAGGPI | |
| | RTEFTSM | |
| 46 | MLEYALKQERAKYHKLKFGTDLNQGEKKADVSEQVSNGPVESVTLENSP | Homo Sapiens |
| | LVWKEGRQLLRQYLEEVGYTDTILDMRSKRVRSLLGRSLELNGAVEPSE | |
| | GAPRAPPGPAGLSGGESLLVKQIEEQIKRNAAGKDGKERLGGSVLGQIP | |
| | FLQNCEDEDSDEDDELDSVQHKKQRVKLPSKALVPEMEDEDEEDDSEDA | |
| | INEFDFLGSGEDGEGAPDPRRCTVDGSPHELESRRVKLQGILADLRDVD | |
| | GLPPKVTGPPPGTPQPRPHEGKRHPPPGPSPAGPWQREAAELSPGLLCL | |
| | QGRGPASSLQPPCPKSSSVCLSPSLFVCPALSVSLSLSSCDSSSCPLPV | |
| | SLSCSLSLSLPLSVILSLPGPLCPSLPSPYQVPLASPQTSSSWTLSGAG | |
| | R | |
| 47 | MRWRTILLQYCFLLITCLLTALEAVPIDIDKTKVQNIHPVESAKIEPPD | Homo Sapiens |
| | TGLYYDEYLKQVIDVLETDKHFREKLQKADIEEIKSGRLSKELDLVSHH | |
| | VRTKLDELKRQEVGRLRMLIKAKLDSLQDIGMDHQALLKQFDHLNHLNP | |
| | DKFESTDLDMLIKAATSDLEHYDKTRHEEFKKYEMMKEHERREYLKTLN | |
| | EEKRKEEESKFEEMKKKHENHPKVNHPGSKDQLKEVWEETDGLDPNDFD | |
| | PKTFFKLHDVNSDGFLDEQELEALFTKELEKVYDPKNEEDDMVEMEEER | |
| | LRMREHVMNEVDTNKDRLVTLEEFLKATEKKEFLEPDSWETLDQQQFFT | |
| | EEELKEYENIIALQENELKKKADELQKQKEELQRQHDQLEAQKLEYHQF | |
| | QDLRMEELWRLKVEDGSPFQGQ | |
| 48 | MFFLWFLRLYLHYLGQWLFLQAISTPVTKFHFSLHIVELCYPTSSLHIG | Homo Sapiens |
| | EELPVVVMGPLMLNAILLLLVLIRWGCQLLFASCPDVLSKLIITMGLWT | |
| | ILDPLAVFILDTLLGRLTDNEETPVADAAKLYWMFVRTVQPGILGVVIT | |
| | VLLYILLFVISSLILYLYCLRLHNDSWILDAFQRIHSEETKFFIPYDLE | |
| | ISNQELSYIVK | |
| 49 | MPSSMGGGGGGSPSPVELRGALVGSVDPTLREQQLQQELLALKQQQQLQ | Homo Sapiens |
| | KQLLFAEFQKQHDHLTRQHEVQLQKHLKQQQEMLAAKQQQEMLAAKRQQ | |
| | ELEQQRQREQQRQEELEKQRLEQQLLILRNKEKSKESAIASTEVKLRLQ | |
| | EFLLSKSKEPTPGGLNHSLPQHPKCWGAHHASLDQSSPPQSGPPGTPPS | |
| | YKLPLPGPYDSRDDFPLRKTASEPNLKVRSRLKQKVAERRSSPLLRRKD | |
| | GTVISTFKKRAVEITGAGPGASSVCNSAPGSGPSSPNSSHSTIAENGFT | |
| | GSVPNIPTEMLPQHRALPLDSSPNQFSLYTSPSLPNISLGLQATVTVTN | |
| | SHLTASPKLSTQQEAERQALQSLROGGTLTGKFMSTSSIPGCLLGVALE | |
| | GDGSPHGHASLLQHVLLLEQARQQSTLIAVPLHGQSPLVTGERVATSMR | |
| | TVGKLPRHRPLSRTQSSPLPQSPQALQQLVMQQQHQQFLEKQKQQQLQL | |
| | GKILTKTGELPRQPTTHPEETEEELTEQQEVLLGEGALTMPREGSTESE | |
| | STQEDLEEEDEEDDGEEEEDCIQVKDEEGESGAEEGPDLEEPGAGYKKL | |
| | FSDAQPLQPLQVYQAPLSLATVPHQALGRTQSSPAAPGGMKSPPDQPVK | |
| | HLFTTGVVYDTFMLKHQCMCGNTHVHPEHAGRIQSIWSRLQETGLLSKC | |
| | ERIRGRKATLDEIQTVHSEYHTLLYGTSPLNRQKLDSKKLLGPISQKMY | |
| | AVLPCGGIGVDSDTVWNEMHSSSAVRMAVGCLLELAFKVAAGELKNGFA | |
| | IIRPPGHHAEESTAMGFCFFNSVAITAKLLQQKLNVGKVLIVDWDIHHG | |
| | NGTQQAFYNDPSVLYISLHRYDNGNFFPGSGAPEEVGGGPGVGYNVNVA | |
| | WTGGVDPPIGDVEYLTAFRTVVMPIAHEFSPDVVLVSAGFDAVEGHLSP | |
| | LGGYSVTARCFGHLTRQLMTLAGGRVVLALEGGHDLTAICDASEACVSA | |
| | LLSVEANTGAVCRSSPLVWAGPCERPKQVRPRRPRL | |
| 50 | MSSVSPIQIPSRLPLLLTHEGVLLPGSTMRTSVDSARNLQLVRSRLLKG | Homo Sapiens |
| | TSLQSTILGVIPNTPDPASDAQDLPPLHRIGTAALAVQVVGSNWPKPHY | |
| | TLLITGLCRFQIVQVLKEKPYPIAEVEQLDRLEEFPNTCKMREELGELS | |
| | EQFYKYAVQLVEMLDMSVPAVAKLRRLLDSLPREALPDILTSIIRTSNK | |
| | EKLQILDAVSLEERFKMTIPLLVRQIEGLKLLQKTRKPKQDDDKRVIAI | |
| | RPIRRITHISGTLEDEDEDEDNDDIVMLEKKIRTSSMPEQAHKVCVKEI | |
| | KRLKKMPQSMPEYALTRNYLELMVELPWNKSTTDRLDIRAARILLDNDH | |
| | YAMEKLKKRVLEYLAVRQLKNNLKGPILCFVGPPGVGKTSVGRSVAKTL | |
| | GREFHRIALGGVCDQSDIRGHRRTYVGSMPGRIINGLKTVGVNNPVFLL | |
| | DEVDKLGKSLQGDPAAALLEVLDPEQNHNFTDHYLNVAFDLSQVLFIAT | |
| | ANTTATIPAALLDRMEIIQVPGYTQEEKIEIAHRHLIPKQLEQHGLTPQ | |
| | QIQIPQVTTLDIITRYTREAGVRSLDRKLGAICRAVAVKVAEGQHKEAK | |
| | LDRSDVTEREGCREHILEDEKPESISDTTDLALPPEMPILIDFHALKDI | |
| | LGPPMYEMEVSQRLSQPGVAIGLAWTPLGGEIMFVEASRMDGEGQLTLT | |
| | GQLGDVMKESAHLAISWLRSNAKKYQLTNAFGSFDLLDNTDIHLHFPAG | |
| | AVTKDGPSAGVTIVTCLASLFSGRLVRSDVAMTGEITLRGLVLPVGGIK | |
| | DKVLAAHRAGLKQVIIPRRNEKDLEGIPGNVRQDLSFVTASCLDEVLNA | |
| | AFDGGFTVKTRPGLLNSKLGRKYQKGLNRQQANMLPPTERVLGWQTDGC | |
| | LIFCETEVLNTGQKMFDCHENTWK | |
| 51 | MARASLCGKEHTPEMWTRPPQEGPCLEVEINENLPARKT | Homo Sapiens |
| 52 | MEAWRGYVLIHGYTARKWKSWDPKPTHLTRTRPWAWQRQLPDTPPYSAS | Homo Sapiens |
| | DSCSPPQVKGECDPPSAHLALLLFLLDSGPCSCDAAHAPAAEHLWNGRL | |
| | LPNPRRLSQLPLKRQSSCHPPGPIRVLPSGDRLFLPSAASVSQPWSLIA | |
| | SNQEEKVHAGTGGLRGMPSVGLPLQTDDK | |
| 53 | MDVVGENEALQQFFEAQGANGTLENPALDTSLLEEFLGNDFDLGAFCSC | Homo Sapiens |
| | DAAHAPAAEHLWNGRLLPNPRRLSQLPLKRQSSCHPPGPIRVLPSGDRL | |
| | FLPSAASVSQPWSLIASNQEEKVHAGTGGLRGMPSVGLPLQTDDK | |
| 54 | MEAWRGYVLIHGYTARKWKSWDPKPTHLTRTRPWACCSCDAAHAPAAEH | Homo Sapiens |
| | LWNGRLLPNPRRLSQLPLKRQSSCHPPGPIRVLPSGDRLFLPSAASVSQ | |
| | PWSLIASNQEEKVHAGTGGLRGMPSVGLPLQTDDK | |
| 55 | MEAWRGYVLIHGYTARKWKSWDPKPTHLTRTRPWAWCMLPNPEAHSWED | Homo Sapiens |
| | SSSFSPPHSCSCDAAHAPAAEHLWNGRLLPNPRRLSQLPLKRQSSCHPP | |
| | GPIRVLPSGDRLFLPSAASVSQPWSLIASNQEEKVHAGTGGLRGMPSVG | |
| | LPLQTDDK | |
| 56 | MAMQKIFAREILDSRGNPTVEVDLHTAKGRFRAAVPSGASTGIYEALEL | Homo Sapiens |
| | RDGDKGRYLGKGVLKAVENINNTLGPALLQKASGEARSLQPPPHAPAPS | |
| | AQTGLSRNIFPYPSPACALTSEKSDLCSPFSNSPFQKLSVVDQEKVDKF | |
| | MIELDGTENKSKFGANAILGVSLAVCKAGAAEKGVPLYRHIADLAGNPD | |
| | LILPVPAFNVINGGSHAGNKLAMQEFMILPVGASSFKEAMRIGAEVYHH | |
| | LKGVIKAKYGKDATNVGDEGGFAPNILENNEALELLKTAIQAAGYPDKV | |
| | VIGMDVAASEFYRNGKYDLDFKSPDDPARHITGEKLGELYKSFIKNYPG | |
| | EAFGCPSVPARIPCSCLIY | |
| 57 | MTTAGRGNLGLIPRSTAFQKQEGRLTVKQEPANQTWGQGSSLQKNYPPV | Homo Sapiens |
| | CEIFRLHFRQLCYHEMSGPQEALSRLRELCRWWLMPEVHTKEQILELLV | |
| | LEQFLSILPGELRTWVQLHHPESGEEAVAVVEDFQRHLSGSEEVRT | |
| 58 | MSQRAKLRSRENQPTVFLPSPDSTLRKYYGEKIGIYFAWLGYYTQMLLL | Homo Sapiens |
| | AAVVGVACFLYGYLNQDNCTWSKEVCHPDIGGKIIMCPQCDRLCPFWKL | |
| | NITCESSKKLCIFDSFGTLVFAVFMGVWVTLFLEFWKRRQAELEYEWDT | |
| | VELQQEEQARPEYEARCTHVVINEITQEEERIPFTAWGKCIRITLCASA | |
| | VFFWILLIIASVIGIIVYRLSVFIVFSAKLPKNINGTDPIQKYLTPQTA | |
| | TSITASIISFIIIMILNTIYEKVAIMITNFELPRTQTDYENSLTMKMFL | |
| | FQFVNYYSSCFYIAFFKGKFVGYPGDPVYWLGKYRNEECDPGGCLLELT | |
| | TQLTIIMGGKAIWNNIQEVLLPWIMNLIGRFHRVSGSEKITPRWEQDYH | |
| | LQPMGKLGLFYEYLEMIIQFGFVTLFVASFPLAPLLALVNNILEIRVDA | |
| | WKLTTQFRRLVPEKAQDIGAWQPIMQGIAILAVVTNAMIIAFTSDMIPR | |
| | LVYYWSFSVPPYGDHTSYTMEGYINNTLSIFKVADFKNKSKGNPYSDLG | |
| | NHTTCRYRDFRYPPGHPQEYKHNIYYWHVIAAKLAFIIVMEHVIYSVKF | |
| | FISYAIPDVSKRTKSKIQREKYLTQKLLHENHLKDMTKNMGVIAERMIE | |
| | AVDNNLRPKSE | |
| 59 | MAERRAFAQKISRTVAAEVRKQISGQYSGSPQLLKNLNIVGNISHHTTV | Homo Sapiens |
| | PLTEAVDPVDLEDYLITHPLAVDSGPLRDLIEFPPDDIEVVYSPRDCRT | |
| | LVSAVPEESEMDPHVRDCIRSYTEDWAIVIRKYHKLGTGFNPNTLDKQK | |
| | ERQKGLPKQVFESDEAPDGNSYQDDQDDLKRRSMSIDDTPRGSWACSIF | |
| | DLKNSLPDALLPNLLDRTPNEEIDRQNDDQRKSNRHKELFALHPSPDEE | |
| | EPIERLSVPDIPKEHFGQRLLVKCLSLKFEIEIEPIFASLALYDVKEKK | |
| | KISENFYFDLNSEQMKGLLRPHVPPAAITTLARSAIFSITYPSQDVFLV | |
| | IKLEKVLQQGDIGECAEPYMIFKEADATKNKEKLEKLKSQADQFCQRLG | |
| | KYRMPFAWTAIHLMNIVSSAGSLERDSTEVEISTGERKGSWSERRNSSI | |
| | VGRRSLERTTSGDDACNLTSFRPATLTVTNFFKQEGDRLSDEDLYKFLA | |
| | DMRRPSSVLRRLRPITAQLKIDISPAPENPHYCLTPELLQVKLYPDSRV | |
| | FMYGEDPSNAMPVIFGKSSCSEFSKEAYTAVVYHNRSPDFHEEIKVKLP | |
| | ATLTDHHHLLFTFYHVSCQQKQNTPLETPVGYTWIPMLQNGRLKTGQFC | |
| | LPVSLEKPPQAYSVLSPEVPLPGMKWVDNHKGVFNVEVVAVSSIHTQDP | |
| | YLDKFFALVNALDEHLFPVRIGDMRIMENNLENELKSSISALNSSQLEP | |
| | VVRFLHLLLDKLILLVIRPPVIAGQIVNLGQASFEAMASIINRLHKNLE | |
| | GNHDQHGRNSLLASYIHYVFRLPNTYPNSSSPGYGSKL | |
| 60 | MAPRGRKRKAEAAVVAVAEKREKLANGGEGMEEATVVIEHCTSVRSSGL | Homo Sapiens |
| | GLRRGPHANSNSLSLKRWWKS | |
| 61 | MQRCPGPLGRGDPPSRKLGLVSVPLQPQGLARMLGAPHPGDSAHQGLRG | Homo Sapiens |
| | GGSPGTWEAGPPAPWTPTQTPSQPRHFPRARGQPGSPGLREGRVWGAGR | |
| | RHIPLLMMPPQSYNLDSRRSCPFPPSPVPGGSPDPFREDHGP | |

What is claimed is:

1. A computer-implemented method for identifying one or more cell surface antigen sequences resulting from alternative splicing in a cell, comprising the steps of:
(a) obtaining a first RNA-seq data set from a first sample cell and a second RNA-seq data set from a second sample cell;
(b) assembling full length mRNA transcript sequences and extracting genomic loci coordinates of the mRNA transcript sequences;
(c) clustering of full length mRNA transcript sequences encoded at the same genomic loci and extraction of exon duo or exon trio mRNA sequences;
(d) selecting the most representative full length mRNA transcript sequences;
(e) identifying stable full length mRNAs transcripts;
(f) translating, in silico the stable full length mRNA transcripts into protein isoform sequences;
(g) identifying protein isoform sequences that are predicted to be stable;
(h) determining B cell antibody accessibility of the protein isoform sequences by using an algorithm to classify the polarity, hydrophobicity, and surface accessibility of peptides derived from the protein isoform sequences;
(i) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic;
(j) generating a first set of antigenic cell surface antigen sequences based on the first RNA-seq data set and a second set of antigenic cell surface antigen sequences based on the second RNA-seq data set ranked by B cell antibody accessibility and T cell antigenicity; and
(k) determining unique antigenic cell surface antigen sequences by comparing the first set of antigenic cell surface antigen sequences and the second set of antigenic cell surface antigen sequences and selecting cell surface antigen sequences present in one set and not the other set;
thereby selecting one or more unique cell surface antigen sequences.

2. A computer-implemented method for identifying one or more cell surface antigen sequences resulting from alternative splicing in a cell, comprising the steps of:
(a) obtaining a first RNA-seq data set from a first sample cell and a second RNA-seq data set from a second sample cell;
(b) assembling full length mRNA transcript sequences and extracting genomic loci coordinates of the mRNA transcript sequences;
(c) clustering of full length mRNA transcript sequences encoded at the same genomic loci and extraction of exon duo or exon trio mRNA sequences;
(d) selecting the most representative full length mRNA transcript sequences;
(e) identifying stable full length mRNAs transcripts;
(f) translating, in silico the stable full length mRNA transcripts into protein isoform sequences;
(g) identifying protein isoform sequences that are predicted to be stable;
(h) determining membrane topologies for each protein isoform;
(i) filtering for membrane bound protein isoform sequences;
(j) determining B cell antibody accessibility of the protein isoform sequences by using an algorithm to classify the polarity, hydrophobicity, and surface accessibility of peptides derived from the protein isoform sequences;
(k) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic;
(l) generating a first set of antigenic cell surface antigen sequences based on the first RNA-seq data set and a second set of antigenic cell surface antigen sequences based on the second RNA-seq data set ranked by B cell antibody accessibility and IF cell antigenicity; and
(m) determining unique antigenic cell surface antigen sequences by comparing the first set of antigenic cell surface antigen sequences and the second set of antigenic cell surface antigen sequences and selecting cell surface antigen sequences present in one set and not the other set;
thereby selecting one or more unique cell surface antigen sequences.

3. The method of claim 1 or claim 2, wherein the semi-supervised or supervised machine learning algorithm comprises: a random forest, Bayesian model, a regression model, a neural network, a classification tree, a regression tree, discriminant analysis, a k-nearest neighbors method, a naive Bayes classifier, support vector machines (SVM), a generative model, a low-density separation method, a graph-based method, a heuristic approach, or a combination thereof.

4. The method of any one of claims 1-3, wherein the machine learning algorithm comprises a random forest algorithm.

5. The method of claim 2, wherein the determining membrane topologies comprises using semi-supervised or supervised machine learning algorithm to classify the membrane topology of the protein isoform, wherein the machine learning algorithm is trained using a training data set comprising training protein sequences encoded with two characteristics i) transmembrane or globular or ii) with signal peptide or without signal peptide.

6. The method of any one of claims 1-5, wherein the cell surface antigen is derived from alternative splicing events selected from the group of intron retention, frameshift, translated lncRNA, novel splicing junction, novel exon, and chimeric.

7. The method of any one of claims 1-6, wherein selecting one or more unique cell surface antigen sequences comprises selecting cell surface antigen sequences that have an increased likelihood of being presented on the tumor cell surface relative to unselected cell surface antigens.

8. The method of any one of claims 1-7, further comprising determining if the cell surface antigen cell surface presentation is MHC-dependent or MHC-independent.

9. The method any one of claims 1-8, wherein the cell surface presentation of the cell surface antigen derived peptide is MHC-independent.

10. The method of any one of claims 1-9, wherein the training peptide sequences comprise peptide sequences having lengths from 5 to 25 amino acids.

11. The method of claim 10, wherein the peptide sequences comprise peptide sequences having lengths from 8 to 15 amino acids.

12. The method of any one of claims 1-11, wherein the training peptide sequences are of viral and bacterial origin.

13. The method of any one of claims 1-12, wherein the first or second cell is a cancer cell.

14. The method of claim 13, wherein the cancer cell is selected from the group consisting of a bone cancer, a breast cancer, a colorectal cancer, a gastric cancer, a liver cancer, a lung cancer, an ovarian cancer, a pancreatic cancer, a prostate cancer, a skin cancer, a testicular cancer, a blood cancer, brain cancer, and a vaginal cancer cell.

15. The method of claim 14, wherein the blood cancer cell is a leukemia, a non-Hodgkin lymphoma, a Hodgkin lymphoma, or a multiple myeloma cell.

16. The method of claim 15, wherein the leukemia cell is Acute Myeloid Leukemia (AML).

17. The method of any one of claims 1-16, wherein the RNA-seq data is obtained by performing sequencing on cells derived from cancer tissue.

18. The method of any one of claims 1-17, wherein the sample cell is derived from a tissue, a blood sample, a cell line, an organoid, saliva, cerebrospinal fluid, or other bodily fluids.

19. The method of any one of claims 1-18, wherein the first cell and the second cell come from the same subject.

20. The method of any one of claims 1-18, wherein the first cell and the second cell come from different subjects.

21. The method of any one of claims 1-20, further comprising generating an output for constructing a personalized cancer vaccine from the selected cell surface antigen.

22. The method of claim 21, wherein the personalized cancer vaccine comprises at least one peptide sequence or at least one nucleotide sequence encoding the selected cell surface antigen.

23. The method of any one of claims 1-22, further comprising receiving information from a user.

24. The method of claim 23, wherein receiving information from a user is via a computer network comprising a cloud network.

25. The method of any one claims 1-24, further comprising a user interface allowing a user to sort membrane topology values, filter B cell accessibility values, filter T cell antigenicity values, select information stored in the database, merge topology values, accessibility values, and antigenicity values with the selected information stored in the database, select cell surface antigen sequences and cell surface antigen derived peptides, or a combination thereof.

26. The method of any one of claims 23-25, further comprising a software module allowing the user to sort, filter, or rank the one or more cell surface antigen sequences or cell surface antigen derived peptides based on user-selected criteria.

27. The method of any one of claims 1-26, further comprising generating an output for constructing a personalized cancer vaccine from the selected cell surface antigen.

28. A method of treating a subject having a cancer, comprising performing any of the steps of claims 1-27, further comprising obtaining a cancer vaccine comprising the selected cell surface antigen, and administering the cancer vaccine to the subject.

29. The method of any one of claims 1-26, further comprising generating an antibody, ADC, or CAR-T cell that specifically binds the selected peptide.

30. A method of treating a subject having a cancer, comprising performing any of the steps of claim 1-26, or 29, further comprising obtaining the antibody, ADC, or CAR-T cell that specifically binds the selected peptide, and administering the antibody; ADC, or CAR-T to the subject.

31. The method of claim 1-26, further comprising generating a TCR engineered T cell that specifically binds the selected peptide.

32. A method of treating a subject having a cancer, comprising performing the steps of any one of claim 1-26, or 31, and further comprising obtaining the TCR engineered T cell that specifically binds the selected peptide, and administering the TCR engineered T cell to the subject.

33. An isolated peptide comprising a cell surface antigen comprising a sequence set forth in Table 1, wherein the peptide is no more than 100 amino acids in length, and an optional pharmaceutically acceptable carrier.

34. The isolated peptide of claim 33, wherein the peptide is no more than 30 amino acids in length or 20 amino acids in length.

35. The isolated peptide of any one of claims 33-34, wherein the amino acid sequence of the peptide consists essentially of or consists of an amino acid sequence set forth in Table 1.

36. The isolated peptide of any one of claims 33-35, wherein the peptide comprises an amino acid sequence set forth in Table 1 and is presentable by a major histocompatibility complex (MHC) Class I or MHC Class II.

37. A recombinant cell engineered to express one or more peptides comprising the amino acid sequences set forth in Table 1 and Table 2.

38. A pharmaceutical composition comprising the peptide of any one of claims 30-36 and a pharmaceutically acceptable carrier or excipient.

39. A pharmaceutical composition comprising a plurality of peptides (e.g., 2, 3, 4, 5, 6, 7, 8, 9, 10, or more) of any one of claims 33-36 and a pharmaceutically acceptable carrier or excipient.

40. A pharmaceutical composition comprising a nucleic acid encoding the peptide of any one of claims 33-36, and a pharmaceutically acceptable carrier or excipient.

41. A pharmaceutical composition comprising one or more nucleic acids encoding a plurality of peptides (e.g., 2, 3, 4, 5, 6, 7, 8, 9, 10, or more) of any one of claims 33-36, and a pharmaceutically acceptable carrier or excipient.

42. The pharmaceutical composition of any one of claims 38-41, further comprising a liposome, wherein the peptide or nucleic acid encoding the peptide is disposed within the liposome.

43. The pharmaceutical composition of any one of claims 38-41, further comprising a lipid nanoparticle, wherein the peptide or nucleic acid encoding the peptide is disposed within the lipid nanoparticle.

44. The pharmaceutical composition of any one of claims 38-43, wherein the peptide or nucleic acid is synthetic.

45. A vaccine that stimulates a T cell mediated immune response when administered to a subject, the vaccine comprising the pharmaceutical composition of any one of claims 38-44.

46. The vaccine of claim 45, wherein the vaccine is a priming vaccine and/or a booster vaccine.

47. A method of determining whether a subject has cancer, the method comprising detecting the presence and/or amount of (i) one or more peptides of any of claims 33-36 and/or (ii) T cells reactive with one or more peptides of any of claims 33-36, in a sample harvested from the subject thereby to determine whether the subject has cancer.

48. The method of claim 46, further comprising selecting a treatment regimen based upon the detected presence or amount of peptide.

49. The method of any one of claims 46-47, wherein the presence or amount of the peptide is determined using RNA-seq, anti-peptide Antibodies, mass spectrometry, tetramer assays, or a combination thereof.

50. The method of any one of claims 46-47, wherein the presence or amount of the T cells is determined by a PCR reaction, tetramer assay, Enzyme Linked Immuno Spot Assay (ELISpot), or an Activation Induced Marker (AIM) assay.

51. The method of any one of claims 46-49, wherein the sample is a tissue, a blood sample, a cell line, an organoid, saliva, cerebrospinal fluid, or other bodily fluids harvested from the subject.

52. A method of treating a cancer in a subject, the method comprising administering a pharmaceutical composition according to any one of claims 38-44 or a vaccine according to claim 45 or claim 46 to the subject.

53. The method of claim 52, wherein the cancer is selected from the group consisting of a bone cancer, a breast cancer, a colorectal cancer, a gastric cancer, a liver cancer, a lung cancer, an ovarian cancer, a pancreatic cancer, a prostate cancer, a skin cancer, a testicular cancer, a blood cancer, brain cancer, and a vaginal cancer.

54. The method of claim 53, wherein the blood cancer is a leukemia, a non-Hodgkin lymphoma, a Hodgkin lymphoma, or a multiple myeloma.

55. The method of claim 54, wherein the leukemia is Acute Myeloid Leukemia (AML).

56. The method of any one of claims 52-55, wherein the composition is administered parenterally.

57. The method of claim 52-55, wherein the composition is administered intravenously.

58. A computer implemented system for identifying one or more cell surface antigen sequences resulting from alternative splicing in a cell, comprising:
a digital processing device comprising a processor, an operating system configured to perform executable instructions, a memory, and a computer program including instructions executable by the digital processing device to create a cell surface antigen analysis application, the application comprising a software module for:
(a) obtaining a first RNA-seq data set from a first sample cell and a second RNA-seq data set from a second sample cell;
(b) assembling full length mRNA transcript sequences and extracting genomic loci coordinates of the mRNA transcript sequences;
(c) clustering of full length mRNA transcript sequences encoded at the same genomic loci and extraction of exon duo or exon trio mRNA sequences;
(d) selecting the most representative full length mRNA transcript sequences;
(e) identifying stable full length mRNAs transcripts;
(f) translating, in silico the stable full length mRNA transcripts into protein isoform sequences;
(g) identifying protein isoform sequences that are predicted to be stable;
(h) determining B cell antibody accessibility of the protein isoform sequences by using an algorithm to classify the polarity, hydrophobicity, and surface accessibility of peptides derived from the protein isoform sequences;
(i) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic;
(j) generating a first set of antigenic cell surface antigen sequences based on the first RNA-seq data set and a second set of antigenic cell surface antigen sequences based on the second RNA-seq data set ranked by B cell antibody accessibility and T cell antigenicity; and
(k) determining unique antigenic cell surface antigen sequences by comparing the first set of antigenic cell surface antigen sequences and the second set of antigenic cell surface antigen sequences and selecting cell surface antigen sequences present in one set and not the other set;
thereby selecting one or more unique cell surface antigen sequences.

59. A computer implemented system for identifying one or more cell surface antigen sequences resulting from alternative splicing in a cell, comprising:
a digital processing device comprising a processor, an operating system configured to perform executable instructions, a memory, and a computer program including instructions executable by the digital processing device to create an cell surface antigen analysis application, the application comprising a software module for:
(a) obtaining a first RNA-seq data set from a first sample cell and a second RNA-seq data set from a second sample cell;
(b) assembling full length mRNA transcript sequences and extracting genomic loci coordinates of the mRNA transcript sequences;
(c) clustering of full length mRNA transcript sequences encoded at the same genomic loci and extraction of exon duo or exon trio mRNA sequences;
(d) selecting the most representative full length mRNA transcript sequences;
(e) identifying stable full length mRNAs transcripts;
(f) translating, in silico the stable full length mRNA transcripts into protein isoform sequences;
(g) identifying protein isoform sequences that are predicted to be stable;
(h) determining membrane topologies for each protein isoform;
(i) filtering for membrane bound protein isoform sequences;
(j) determining B cell antibody accessibility of the protein isoform sequences by using an algorithm to classify the polarity, hydrophobicity, and surface accessibility of peptides derived from the protein isoform sequences;
(k) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic;
(l) generating a first set of antigenic cell surface antigen sequences based on the first RNA-seq data set and a second set of antigenic cell surface antigen sequences based on the second RNA-seq data set ranked by B cell antibody accessibility and T cell antigenicity; and
(m) determining unique antigenic cell surface antigen sequences by comparing the first set of antigenic cell surface antigen sequences and the second set of antigenic cell surface antigen sequences and selecting cell surface antigen sequences present in one set and not the other set;
thereby selecting one or more unique cell surface antigen sequences.

60. The system of claim 58 or claim 59, wherein the semi-supervised or supervised machine learning algorithm comprises: a random forest, Bayesian model, a regression model, a neural network, a classification tree, a regression tree, discriminant analysis, a k-nearest neighbors method, a naive Bayes classifier, support vector machines (SVM), a generative model, a low-density separation method, a graph-based method, a heuristic approach, or a combination thereof.

61. The system of any one of claims 58-60, wherein the machine learning algorithm comprises a random forest algorithm.

62. The system of claim 61, wherein the determining membrane topologies comprises using semi-supervised or supervised machine learning algorithm to classify the membrane topology of the protein isoform, wherein the machine learning algorithm is trained using a training data set comprising training protein sequences encoded with two characteristics i) transmembrane or globular or ii) with signal peptide or without signal peptide.

63. The system of any one of claims 58-62, wherein the cell surface antigen is derived from alternative splicing events selected from the group of intron retention, frameshift, translated lncRNA, novel splicing junction, novel exon, and chimeric.

64. The system of any one of claims 58-63, wherein selecting the set of peptides comprises selecting peptides that have an increased likelihood of being presented on the tumor cell surface relative to unselected peptides.

65. The system of any one of claims 58-64, further comprising determining if the cell surface antigen cell surface presentation is MHC-dependent or MHC-independent.

66. The system of claim 65, wherein the cell surface presentation of the cell surface antigen derived peptide is MHC-independent.

67. The system of any one of claims 58-66, wherein the training peptide sequences comprise peptide sequences having lengths from 5 to 25 amino acids.

68. The system of claim 67, wherein the peptide sequences comprise peptide sequences having lengths from 8 to 15 amino acids.

69. The system of any one of claims 58-68, wherein the training peptide sequences are of viral and bacterial origin.

70. The system of any one of claims 58-69, wherein the first or second cell is a cancer cell.

71. The system of claim 70, wherein the wherein the cancer cell is selected from the group consisting of a bone cancer, a breast cancer, a colorectal cancer, a gastric cancer, a liver cancer, a lung cancer, an ovarian cancer, a pancreatic cancer, a prostate cancer, a skin cancer, a testicular cancer, a blood cancer, brain cancer, and a vaginal cancer cell.

72. The system of claim 71, wherein the blood cancer cell is a leukemia, a non-Hodgkin lymphoma, a Hodgkin lymphoma, or a multiple myeloma cell.

73. The system of claim 72, wherein the leukemia cell is Acute Myeloid Leukemia (AML).

74. The system of any one of claims 58-73, wherein the RNA-seq data is obtained by performing sequencing on cells derived from cancer tissue.

75. The system of any one of claims 58-74, wherein the sample cell is derived from a tissue, a blood sample, a cell line, an organoid, saliva, cerebrospinal fluid, or other bodily fluids.

76. The system of any one of claims 58-75, further comprising generating an output for constructing a personalized cancer vaccine from the selected cell surface antigen.

77. The system of claim 76, wherein the personalized cancer vaccine comprises at least one peptide sequence or at least one nucleotide sequence encoding the selected cell surface antigen.

78. The system of any one of claims 58-77, further comprising receiving information from a user.

79. The system of claim 78, wherein receiving information from a user is via a computer network comprising a cloud network.

80. The system of any one claims 58-79, further comprising a user interface allowing a user to sort membrane topology values, filter B cell accessibility values, filter T cell antigenicity values, select information stored in the database, merge topology values, accessibility values, and antigenicity values with the selected information stored in the database, select cell surface antigen sequences and cell surface antigen derived peptides, or a combination thereof.

81. The system of any one of claims 78-80, further comprising a software module allowing the user to sort, filter, or rank the one or more cell surface antigen sequences or cell surface antigen derived peptides based on user-selected criteria.

82. The system of any one of claims 58-81, further comprising generating an output for constructing a personalized cancer vaccine from the selected cell surface antigen.

83. The system of claim 82, wherein the personalized cancer vaccine comprises at least one peptide sequence or at least one nucleotide sequence encoding the selected cell surface antigen.

84. A computer-implemented method for identifying a disease-specific cell surface antigen or cell surface antigen derived peptide comprising:
(a) obtaining a first RNA-seq data set from a first sample cell and a second RNA-seq data set from a second sample cell;
(b) assembling full length mRNA transcript sequences and extracting genomic loci coordinates of the mRNA transcript sequences;
(c) clustering of full length mRNA transcript sequences encoded at the same genomic loci and extraction of exon duo or exon trio mRNA sequences;
(d) selecting the most representative full length mRNA transcript sequences;
(e) identifying stable full length mRNAs transcripts;
(f) translating, in silico the stable full length mRNA transcripts into protein isoform sequences;
(g) identifying protein isoform sequences that are predicted to be stable;
(h) determining B cell antibody accessibility of the protein isoform sequences by using an algorithm to classify the polarity, hydrophobicity, and surface accessibility of peptides derived from the protein isoform sequences;
(i) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic;
(j) generating a first set of antigenic cell surface antigen sequences based on the first RNA-seq data set and a second set of antigenic cell surface antigen sequences based on the second RNA-seq data set ranked by B cell antibody accessibility and T cell antigenicity; and
(k) determining unique cell surface antigen sequences by comparing the first set of antigenic cell surface antigen sequences and the second set of antigenic cell surface antigen sequences and selecting cell surface antigen sequences present in the second set and not the first set;
thereby selecting one or more unique cell surface antigen sequences unique in the second set that are disease specific.

85. A computer-implemented method for identifying a disease-specific cell surface antigen comprising:
(a) obtaining a first RNA-seq data set from a first sample cell and a second RNA-seq data set from a second sample cell;
(b) assembling full length mRNA transcript sequences and extracting genomic loci coordinates of the mRNA transcript sequences;
(c) clustering of full length mRNA transcript sequences encoded at the same genomic loci and extraction of exon duo or exon trio mRNA sequences;
(d) selecting the most representative full length mRNA transcript sequences;
(e) identifying stable full length mRNAs transcripts;
(f) translating, in silico the stable full length mRNA transcripts into protein isoform sequences;
(g) identifying protein isoform sequences that are predicted to be stable;
(h) determining membrane topologies for each protein isoform;
(i) filtering for membrane bound protein isoform sequences;
(j) determining B cell antibody accessibility of the protein isoform sequences by using an algorithm to classify the polarity, hydrophobicity, and surface accessibility of peptides derived from the protein isoform sequences;
(k) determining T cell antigenicity of the protein isoform sequences by using a semi-supervised or supervised machine learning algorithm, wherein the semi-supervised or supervised machine learning algorithm is trained using a training data set comprising training peptide sequences encoded with two characteristics (i) responsive or non-responsive, and/or (ii) antigenic or non-antigenic;
(l) generating a first set of antigenic cell surface antigen sequences based on the first RNA-seq data set and a second set of antigenic cell surface antigen sequences based on the second RNA-seq data set ranked by B cell antibody accessibility and T cell antigenicity; and
(m) determining unique cell surface antigen sequences by comparing the first set of antigenic cell surface antigen sequences and the second set of antigenic cell surface antigen sequences and selecting cell surface antigen sequences present in the second set and not the first set;
thereby selecting one or more unique cell surface antigen sequences unique in the second set that are disease specific.

86. The method of claim 84 or 85, wherein the diseased sample cell is a cancer cell.
